(19) United States
(12) Patent Application Publication
(10) Pub. No.: US 2022/0403035 A1
(43) Pub. Date: Dec. 22, 2022

(54) MULTITARGETING ANTIGEN-BINDING MOLECULES FOR USE IN PROLIFERATIVE DISEASES

(71) Applicants: AMGEN RESEARCH ( MUNICH) GMBH, Munich (DE); AMGEN INC., Thousand Oaks, CA (US)

(72) Inventors: Jonas Anlahr, Munich (DE); Tara Arvedson, Thousand Oaks, CA (US); Julie Bailis, Thousand Oaks, CA (US); Matthew Chun, Thousand Oaks, CA (US); Igor D'Angelo, Thousand Oaks, CA (US); Christopher Dahlhoff, Munich (DE); Stephanie Everts, Munich (DE); Mattias Friedrich, Munich (DE); Lars Gaedtke, Munich (DE); Patrick Hoffmann, Munich (DE); Rebecca Goldstein, Thousand Oaks, CA (US); Jonas Karl-Josef Honer, Munich (DE); Roman Kischel, Munich (DE); Matthias Klinger, Munich (DE); Peter Kufer, Munich (DE); Markus Muenz, Munich (DE)

(21) Appl. No.: 17/774,232

(22) PCT Filed: Nov. 6, 2020
(86) PCT No.: PCT/EP2020/081224

§ 371 Date: May 4, 2022

Related U.S. Application Data
(60) Provisional application No. 62/931,783, filed on Nov. 6, 2019.
(60) Provisional application No. 62/953,120, filed on Dec. 23, 2019.

(51) Int. Cl. C07K 16/28

(52) U.S. Cl. CPC C07K 16/2851; C07K 16/2863; C07K 16/2803; C07K 16/2878; C07K 16/2887; C07K 2317/622; C07K 2317/31; C07K 2317/92; C07K 2317/73; C07K 2317/34; C07K 2317/94

(57) ABSTRACT

The present invention provides bispecific antigen-binding molecules characterized by comprising a first and a second domain, each binding to any of CS1, BCMA, CD20, CD22, FLT3, CD123, CLL1, MSLN, CDH3 or EpCAM, a third domain binding to an extracellular epitope of the human and the Macaca CD3ε chain and optionally a fourth domain, which is a Fc modality. Moreover, the invention provides a polynucleotide, encoding the antigen-binding molecule, a vector comprising this polynucleotide, host cells, expressing the construct and a pharmaceutical composition comprising the same.

### TECHNICAL FIELD

This invention relates to products and methods of biotechnology, in particular to multitargeting antigen-binding molecules, their preparation and their use.

### BACKGROUND

In comparison to small chemical drugs, protein pharmaceuticals have high specificity and activity at relatively low concentrations, and typically provide for therapy of high impact diseases such as various cancers, auto-immune diseases, and metabolic disorders (Roberts, Trends Biotechnol. 2014 July; 32(7):372-80, Wang, Int J Pharm. 1999 Aug. 20; 185(2):129-88). Such new protein-based pharmaceuticals comprise, for example, bispecific (monoclonal) antibodies which typically can simultaneously bind to two different types of antigen. They are known in several structural formats, and current applications have been explored for cancer immunotherapy and drug delivery (Fan, Gaowei; Wang, Zujian; Hao, Mingju; Li, Jinming (2015). “Bispecific antibodies and their applications”. Journal of Hematology & Oncology. 8: 130).

Bispecific molecules useful in immunooncology can be antigen-binding polypeptides such as antibodies, e.g. IgG-like, i.e. full-length bispecific antibodies, or non-IgG-like bispecific antibodies, which are not full-length antigen-binding molecules. Full length bispecific antibodies typically retain the traditional monoclonal antibody (mAb) structure of two Fab arms and one Fc region, except the two Fab sites bind different antigens. Non-full-length bispecific antibodies can lack an Fc region entirely. These include chemically linked Fabs, consisting of only the Fab regions, and various types of bivalent and trivalent single-chain variable fragments (scFvs). There are also fusion proteins mimicking the variable domains of two antibodies. An example of such a format is the bi-specific T-cell engager (BiTE®) (Yang, Fa; Wen, Weihong; Qin, Weijun (2016). “Bispecific Antibodies as a Development Platform for New Concepts and Treatment Strategies”. International Journal of Molecular Sciences. 18 (1): 48).

Exemplary bispecific antibody-derived molecules such as BiTE® molecules are recombinant protein constructs made from two flexibly linked antibody derived binding domains. One binding domain of BiTE® molecules is specific for a selected tumor-associated surface antigen on target cells; the second binding domain is specific for CD3, a subunit of the T cell receptor complex on T cells. By their particular design, BiTE® antigen-binding molecules are uniquely suited to transiently connect T cells with target cells and, at the same time, potently activate the inherent cytolytic potential of T cells against target cells. An important further development of the first generation of BiTE® molecules (see WO 99/54440 and WO 2005/040220) developed into the clinic as AMG 103 and AMG 110 was the provision of bispecific antigen-binding molecules binding to a context independent epitope at the N-terminus of the CD3s chain (WO 2008/119567). BiTE® molecules binding to this elected epitope do not only show cross-species specificity for the human and the Macaca, or Callithrix jacchus, Saguinus oedipus or Saimiri sciureus CD3ε chain, but also, due to recognizing this specific epitope (instead of previously described epitopes of CD3 binders in bispecific T cell engaging molecules), do not demonstrate unspecific activation of T cells to the same degree as observed for the previous generation of T cell engaging antibodies. This reduction in T cell activation was connected with less or reduced T cell redistribution in patients, the latter being identified as a risk for side effects, e.g. in pasotuximab.

Antibody-based molecules as described in WO 2008/119567 are characterized by rapid clearance from the body; thus, while they are able to reach most parts of the body rapidly, their in vivo applications may be limited by their brief persistence in vivo. On the other hand, their concentration in the body can be adapted and fine-tuned at short notice. Prolonged administration by continuous intravenous infusion is used to achieve therapeutic effects because of the short in vivo half-life of this small, single chain molecule. However, bispecific antigen-binding molecules are available which have more favorable pharmacokinetic properties, including a longer half-life as described in WO 2017/134140. An increased half-life is typically useful in in vivo applications of immunoglobulins, especially with respect to especially antibody fragments or constructs of small size, e.g. in the interest of patient compliance.

One challenging ongoing problem in antibody-based immunooncology is tumor escape. Such tumor escape happens when the immune system—even if triggered or directed by some antibody—based immune-therapeutics—is not capable enough to eradicate tumors, which carry accumulated genetic and epigenetic alterations and use several mechanisms to be the victorious of the immunoediting process (Keshavarz-Fathi, Mahsa; Rezaei, Nima (2019) “Vaccines for Cancer Immunotherapy”). Generally, four mechanisms interfering with effective antitumor immune responses are known: (1) defective tumor antigen processing or presentation, (2) lack of activating mechanisms, (3) inhibitory mechanisms and immunosuppressive state, and (4) resistant tumor cells. Especially with respect to the first mechanism, tumor antigens might be present in a new form due to the genetic instability, mutation of the tumor and escape from immune system. Epitope-negative tumor cells remain hidden and consequently resistant to the immune rejection. They have been developed following the elimination of epitope-positive tumor cells, similar to Darwin's theory of natural selection. In consequence, antibody-based immune-therapy directed against an antigen on tumor cells is rendered ineffective when such tumor cells no longer express a respective antigen due to tumor escape. Said antigen loss is understood herein as driving force for tumor escape and thus, used interchangeably. Accordingly, there is a need to provide improved antibody-based immunooncology which addresses the problem of antigen loss to effectively prevent tumor escape.

Further, despite the so-far achieved pre-clinical and clinical success of antibody-based immune-therapeutics, notable limitations remain including differential responses between individuals and cancer types. Not all patients will respond to therapy at available safe doses as dose-limiting toxicity can be a limiting factor for the efficacy of antibody-based immune-therapeutics. Hence, there is also a need to reduce dose-limiting toxicity in antibody-based immune-therapeutics to make such therapy available to more patients suffering from diverse proliferative diseases.

Another challenge to the broad utilization of immunooncology with respect to T-cell engaging bispecific molecules is the availability of suitable targets (Bacac et al., Clin Cancer Res; 22(13) Jul. 1, 2016). For example, solid tumor targets may be overexpressed on tumor cells but expressed at lower, yet significant levels on non-malignant primary cells in critical tissues. In nature, according to Bacac et al, T cells can distinguish between high- and low-antigen expressing cells by means of relatively low-affinity T cell receptors (TCRs) that can still achieve high-avidity binding to target cells expressing sufficiently high levels of target antigen. T-cell engaging bispecific molecules that could facilitate the same, and thus maximize the window between killing of high- and low-target expressing cells, are thus highly desirable. One approach discussed in the art is the use of dual targeting of two antigens on the same cell leads to improved target selectivity over normal tissues that express only one or low levels of both target antigens. This effect is thought to be dependent on the avidity component mediated by the concurrent binding of the bsAb to both antigens on the same cell. With respect to dual targeting as such, some multispecific monoclonal antibodies (mAb) or other immune constructs are known in the art. WO 2014/116846 teaches a multispecific binding protein comprising a first binding site that specifically binds to a target cell antigen, a second binding site that specifically binds to a cell surface receptor on an immune cell, and a third binding site that specifically binds to cell surface modulator on the immune cell. US 2017/0022274 discloses a trivalent T-cell redirecting complex comprising a bispecific antibody, wherein the bispecific antibody has two binding sites against a tumor-associated antigen (TAA) and one binding site against a T-cell. While different multispecific antibodies or antibody fragments are known in the art, some of which address T-cells, no multitargeting bispecific molecules employing the mechanism of a—preferably single chain—bispecific T-cell engaging molecule has been proposed before which both addresses the need of overcoming antigen loss/tumor escape and to reduce dose-limiting toxicity in antibody-based immune-therapeutics while effectively redirecting T-cells by one stable and ready-to-use therapeutic system.

### SUMMARY

In view of the needs described above, it is an object of the present invention to provide multitargeting antigen-binding molecules, typically polypeptides, such as T cell engaging bispecific molecules, which are specifically suitable to bind two antigens on a target cell associated with specific conditions and one antigen on an effector cell at the same time, preferably for use in the treatment of said specific conditions. Accordingly, the present invention provides a multitargeting bispecific antigen-binding molecule characterized by comprising a first domain binding to a target cell surface antigen (e.g. a first TAA), a second domain binding to the same or preferably a different target cell surface antigen (e.g. a second TAA), a third domain binding to an extracellular epitope of the human and non-human, e.g. Macaca CD3ε chain, and preferably a fourth domain, which is a specific Fc modality which modulates half-life of the molecule. Preferably, the domains are binding domains comprised of VH and VL domains in amino to carboxyl orientation, respectively, wherein a flexible but short peptide linker links the VL of the first binding domain to the VH of the second binding domain. Surprisingly, activity of the molecules of the present invention against target cells associated with particular diseases can be preserved thereby without steric hindrance between the first and the second binding domain, and without the requirement of providing long linkers which would disadvantageously be more prone to degradation, cleavage or the like than the instantly provided shorter linkers. Moreover, the invention provides a polynucleotide encoding the antigen-binding molecule, a vector comprising this polynucleotide, and host cells expressing the construct and a pharmaceutical composition comprising the same.

In a first aspect, it is envisaged in the context of the present invention to provide a multispecific antigen-binding molecule comprising at least three binding domains, wherein
(i) the first binding domain comprises a paratope which immuno-specifically binds to a first target cell surface antigen (e.g. TAA1),
(ii) the second binding domain comprises a paratope which immune-specifically binds to a second target cell surface antigen (e.g. TAA2), and
(iii) the third binding domain comprises a paratope which immune-specifically binds to an extracellular epitope of the human and/or the Macaca CD3ε chain,
wherein the first, second and third binding domain are arranged in an amino to carboxyl order, and wherein the first binding domain and the second binding domain are linked by a peptide linker having a length of 5 to 24 amino acids, preferably 5 to 18 amino acids.

Within said aspect, it is also envisaged in the context of the present invention to provide an multispecific antigen-binding molecule, wherein the antigen-binding molecule comprises a fourth domain which comprises two polypeptide monomers, each comprising a hinge, a CH2 and a CH3 domain, wherein said two polypeptide monomers are fused to each other via a peptide linker.

Within said aspect, it is also envisaged in the context of the present invention to provide an multispecific antigen-binding molecule, wherein said forth domain comprises in an amino to carboxyl order:
hinge-CH2-CH3-linker-hinge-CH2-CH3.

Within said aspect, it is also envisaged in the context of the present invention to provide an multispecific antigen-binding molecule, wherein each of said polypeptide monomers in the fourth domain has an amino acid sequence that is at least 90% identical to a sequence selected from the group from the group consisting of: SEQ ID NO: 17-24, wherein preferably each of said polypeptide monomers has an amino acid sequence selected from SEQ ID NO: 17-24.

Within said aspect, it is also envisaged in the context of the present invention to provide an multispecific antigen-binding molecule, wherein the CH2 domain comprises an intra domain cysteine disulfide bridge.

Within said aspect, it is also envisaged in the context of the present invention to provide an multispecific antigen-binding molecule, wherein the first, second, third and the optional fourth binding domain are arranged in an amino to carboxyl order.

Within said aspect, it is also envisaged in the context of the present invention to provide an multispecific antigen-binding molecule, wherein the antigen-binding molecule is a single chain antigen-binding molecule, preferably a multispecific scFv antigen-binding molecule.

Within said aspect, it is also envisaged in the context of the present invention to provide an multispecific antigen-binding molecule, wherein the first, second, and third binding domain each comprise in a amino to carboxyl order a VH domain and a VL domain.

Within said aspect, it is also envisaged in the context of the present invention to provide an multispecific antigen-binding molecule, wherein the peptide linker between the VL of the first binding domain and the VH of the second binding domain is selected from having a length of 5, 6, 7, 8, 9, 10, 11, 12, 13, 14, 15, 16, 17, 18, 19, 20, 21, 22, 23, or 24 amino acids, preferably 5, 6, 7, 8, 9, 10, 11 or 12 amino acids, more preferably 6 amino acids.

Within said aspect, it is also envisaged in the context of the present invention to provide an multispecific antigen-binding molecule, wherein the peptide linker between the VL of the first binding domain and the VH of the second binding domain is a flexible linker which comprises serine and glycine as amino acid building blocks, preferably only serine (Ser, S) and glycine (Gly, G).

Within said aspect, it is also envisaged in the context of the present invention to provide an multispecific antigen-binding molecule, wherein the peptide linker between the first binding domain and the second binding domain is preferably rich in small and/or hydrophilic amino acids and preferably selected from the group consisting of S(G₄S)n, (G₄S)n, (G₄)n, and (G₅)n, wherein n equals 1, 2, 3 or 4, more preferably n equals 1 or 2, more preferably SG₄S.

Within said aspect, it is also envisaged in the context of the present invention to provide an multispecific antigen-binding molecule, wherein any of the first target cell surface antigen and the second target cell surface antigen is selected from the group consisting of CS1, BCMA, FLT3, CD123, CD20, CD22, EpCAM, MSLN, CDH3 and CLL1.

Within said aspect, it is also envisaged in the context of the present invention to provide an multispecific antigen-binding molecule, wherein the first target cell surface antigen and the second target cell surface antigen are not identical.

Within said aspect, it is also envisaged in the context of the present invention to provide an multispecific antigen-binding molecule, wherein the first binding domains is capable of binding to the first target cell surface antigen and the second binding domain is capable of binding to the second target cell surface antigen simultaneously, preferably wherein the first target cell surface antigen and the second target cell surface antigen are on the same target cell.

Within said aspect, it is also envisaged in the context of the present invention to provide an multispecific antigen-binding molecule, wherein the first target cell surface antigen and the second target cell surface antigen, respectively, are selected from the group consisting of CS1 and BCMA, BCMA and CS1, FLT3 and CD123, CD123 and FLT3, CD20 and CD22, CD22 and CD20, EpCAM and MSLN, MSLN and EpCAM, CDH3 and MSN, MSLN and CDH3, FLT3 and CLL1, and CLL1 and FLT3.

Within said aspect it is also envisaged that the multispecific antigen-binding molecule is characterized by
(i) the first and second domain comprise two antibody variable domains and the third domain comprises two antibody variable domains;
(ii) the first and second domain comprise one antibody variable domain and the third domain comprises two antibody variable domains;
(iii) the first and second domain comprise two antibody variable domains and the second domain comprises one antibody variable domain; or
(iv) the first domain comprises one antibody variable domain and the second domain comprises one antibody variable domain.

Within said aspect, it is also envisaged in the context of the present invention to provide an multispecific antigen-binding molecule, wherein the first, second and third domain, which are fused by respective peptide linkers, are fused to the fourth domain via a peptide linker.

Within said aspect, it is also envisaged in the context of the present invention to provide an multispecific antigen-binding molecule, wherein the antigen-binding molecule comprises in an amino to carboxyl order:
(a) the first domain;
(b) a peptide linker preferably having an amino acid sequence selected from the group consisting of SEQ ID NOs: 1-4 and 9-12, preferably 11;
(c) the second domain,
(d) a peptide linker preferably having an amino acid sequence selected from the group consisting of SEQ ID NOs: 1-3; and
(c) the third domain.

Within said aspect, it is also envisaged in the context of the present invention to provide an multispecific antigen-binding molecule, wherein the antigen-binding molecule further comprises in an amino to carboxyl order:
(f) a peptide linker having an amino acid sequence selected from the group consisting of SEQ ID NOs: 1, 2, 3, 9, 10, 11 and 12.
(e) the first polypeptide monomer of the fourth domain;
(f) a peptide linker having an amino acid sequence selected from the group consisting of SEQ ID NOs: 5, 6, 7 and 8; and
(g) the second polypeptide monomer of the fourth domain.

Within said aspect, it is also envisaged in the context of the present invention to provide an antigen-binding molecule, wherein the first and the second binding domain comprise a VH region comprising CDR-H1, CDR-H2 and CDR-H3 selected from—the group consisting of SEQ ID Nos: 33 to 35, 44 to 46, 55 to 57, 66 to 68, 77 to 79, 88 to 90, 99 to 101, 110 to 112, 121 to 123, 132 to 134, 143 to 145, 154 to 156, 165 to 167, 176 to 178, 187 to 189, 198 to 200,209 to 211,220 to 222,231 to 233, 242 to 244, 253 to 255, 264 to 266, 275 to 277, 286 to 288, 297 to 299, 308 to 310, 319 to 321, 330 to 332, 341 to 343, 352 to 354, 363 to 365, 374 to 376, 385 to 387, 396 to 398, 407 to 409, 418 to 420, 429 to 431, 440 to 442, 451 to 453, 462 to 464, 473 to 475, 484 to 486, 495 to 497, 506 to 508, 517 to 519, 528 to 530, 539 to 541, 550 to 552, 561 to 563, 572 to 574, 583 to 585, 594 to 596, 605 to 607, 616 to 618, 627 to 629, 638 to 640, 649 to 651, 660 to 662, 896 to 898, 907 to 909, 918 to 920, 929 to 931, 940 to 942, 951 to 953, 962 to 964, 973 to 975, 984 to 986, 995 to 997, 1006 to 1008, 1017 to 1019, 1028 to 1030, 1039 to 1041, 1050 to 1052, 1061

to 1063, 1072 to 1074, 1083 to 1085, 1094 to 1096, 1105 to 1107, 1116 to 1118, 1127 to 1129, 1138 to 1140, 1149 to 1151, 1160 to 1162, 1171 to 1173, 1182 to 1184, 1193 to 1195, 1204 to 1206, 1215 to 1217, 1226 to 1228, 1237 to 1239, 1248 to 1250, 1259 to 1261, 1270 to 1272, 1281 to 1283, 1292 to 1294, 1303 to 1305, 1314 to 1316, 1325 to 1327, 1336 to 1338, 1347 to 1349, 1358 to 1360, 1369 to 1371, 1380 to 1382, 1391 to 1393, 1402 to 1404, 1413 to 1415, 1424 to 1426, 1489 to 1491, 1500 to 1502, 1511 to 1513, 1522 to 1524, 1533 to 1535, 1544 to 1546, 1555 to 1557, 1566 to 1568, 1577 to 1579, 1588 to 1590, 1599 to 1601, 1610 to 1612, 1621 to 1623, 1632 to 1634, 1643 to 1645, 1654 to 1656, 1827 to 1829, 1840 to 1842, 1853 to 1855, 1866 to 1868, 1879 to 1881, 1892 to 1894, 11905 to 1907, 1922 to 1924, 1935 to 1937, 1948 to 1950, 1961 to 1963, 1974 to 1976, 1987 to 1989, 2000 to 2002, 2013 to 2015, 2026 to 2028, 2039 to 2041, 2052 to 2054, 2065 to 2067, 2078 to 2080, 2091 to 2093, 2104 to 2106, 2117 to 2119, 2130 to 2132, 2143 to 2145, 2156 to 2158, 2169 to 2171, 2182 to 2184, 2195 to 2197, 2208 to 2210, 2221 to 2223, 2234 to 2236, 2247 to 2249, 3346 to 3348, 3357 to 3359, 3368 to 3370, 3379 to 3381, 3390 to 3392, 3401 to 3403, 3412 to 3414, 3423 to 3425, 3434 to 3436, 3445 to 3447, 3456 to 3458, 3467 to 3469, 3478 to 3480, 3489 to 3491, 3500 to 3502, 3511 to 3513, 3522 to 3524, 3533 to 3535, 3544 to 3546, 3555 to 3557, 3566 to 3568, 3679 to 3681, 3690 to 3692, 3712 to 3714, 3723 to 3725.

Within said aspect, it is also envisaged in the context of the present invention to provide an multispecific antigen-binding molecule, wherein the first and second binding domain comprise a VL region comprising CDR-L1, CDR-L2 and CDR-L3 selected from the group consisting of SEQ ID Nos: 36 to 38, 47 to 49, 58 to 60, 69 to 71, 80 to 82, 91 to 93, 102 to 104, 113 to 115, 124 to 126, 135 to 137, 146 to 148, 157 to 159, 168 to 170, 179 to 181, 190 to 192, 201 to 203, 212 to 214, 223 to 225, 234 to 236, 245 to 247, 256 to 258, 267 to 269,278 to 280, 289 to 291, 300 to 302, 311 to 313, 322 to 324, 333 to 335, 344 to 346, 355 to 357, 366 to 368, 377 to 379, 388 to 390, 399 to 401, 410 to 412, 421 to 423, 432 to 434, 443 to 445, 454 to 456, 465 to 467, 476 to 478, 487 to 489, 498 to 500, 509 to 511, 520 to 522, 531 to 533, 542 to 544, 553 to 555, 564 to 566, 575 to 577, 586 to 588, 597 to 599, 608 to 610, 619 to 621, 630 to 632, 641 to 643, 652 to 654, 663 to 665, 899 to 901, 910 to 912, 921 to 923, 932 to 934, 943 to 945, 954 to 956, 965 to 967, 976 to 978, 987 to 989, 998 to 1000, 1009 to 1011, 1020 to 1022, 1031 to 1033, 1042 to 1043, 1053 to 1055, 1064 to 1066, 1075 to 1077, 1086 to 1088, 1097 to 1099, 1108 to 1110, 1119 to 1121, 1130 to 1132, 1141 to 1143, 1152 to 1154, 1163 to 1165, 1174 to 1176, 1185 to 1187, 1196 to 1198, 1207 to 1209, 1218 to 1220, 1229 to 1231, 1240 to 1242, 1251 to 1253, 1262 to 1264, 1273 to 1275, 1284 to 1286, 1295 to 1297, 1306 to 1308, 1317 to 1319, 1328 to 1330, 1339 to 1341, 1350 to 1352, 1361 to 1363, 1372 to 1374, 1383 to 1385, 1394 to 1396, 1405 to 1407, 1416 to 1418, 1427 to 1429, 1492 to 1494, 1503 to 1505, 1514 to 1516, 1525 to 1527, 1536 to 1538, 1547 to 1549, 1558 to 1560, 1569 to 1571, 1580 to 1582, 1591 to 1593, 1602 to 1604, 1613 to 1615, 1624 to 1626, 1635 to 1637, 1646 to 1648, 1657 to 1659, 1830 to 1832, 1843 to 1845, 1856 to 1858, 1869 to 1871, 1882 to 1884, 1895 to 1897, 1908 to 1910, 1925 to 1927, 1938 to 1940,

1951 to 1953, 1964 to 1966, 1977 to 1979, 1990 to 1992, 2003 to 2005, 2016 to 2018, 2029 to 2031, 2042 to 2044, 2055 to 2057, 2068 to 2070, 2081 to 2083, 2094 to 2096, 2107 to 2109, 2120 to 2122, 2133 to 2135, 2146 to 2148, 2159 to 2131, 2172 to 2174, 2085 to 2187, 2198 to 2200, 2211 to 2213, 2224 to 2226, 2237 to 2239, 2250 to 2252, 3349 to 3351, 3360 to 3362, 3371 to 3373, 3382 to 3384, 3393 to 3392, 3404 to 3406, 3415 to 3417, 3426 to 3428, 3437 to 3439, 3448 to 3450, 3459 to 3461, 3470 to 3472, 3481 to 3483, 3492 to 3494, 3503 to 3505, 3514 to 3516, 3525 to 3527, 3536 to 3538, 3547 to 3549, 3558 to 3560, 3569 to 3571, 3682 to 3684, 3693 to 3695, 3715 to 3717, and 3726 to 3728.

Within said aspect, it is also envisaged in the context of the present invention to provide an multispecific antigen-binding molecule, wherein the first and second binding domain comprise a VH region selected from the group consisting of SEQ ID Nos: 39, 50, 61, 72, 83, 94, 105, 116, 127, 138, 149, 160, 171, 182, 193, 204, 215, 226, 237, 248, 259, 270, 281, 292, 303, 314, 325, 336, 347, 358, 369, 380, 391, 402, 413, 424, 435, 446, 457, 468, 479, 490, 501, 512, 523, 534, 545, 556, 567, 578, 589, 600, 611, 622, 633, 644, 655, 666, 902, 913, 924, 935, 946, 957, 968, 979, 990, 1001, 1012, 1023, 1034, 1045, 1056, 1067, 1078, 1089, 1100, 1111, 1122, 1133, 1144, 1155, 1166, 1177, 1188, 1199, 1210, 1221, 1232, 1243, 1254, 1265, 1276, 1287, 1298, 1309, 1320, 1331, 1342, 1353, 1364, 1375, 1386, 1397, 1408, 1419, 1430, 1495, 1506, 1517, 1528, 1539, 1550, 1561, 1572, 1583, 1594, 1605, 1616, 1627, 1638, 1649, 1660, 1833, 1846, 1859, 1872, 1885, 1898, 1911, 1928, 1941, 1954, 1967, 1980, 1993, 2006, 2019, 2032, 2045, 2058, 2071, 2084, 2097, 2110, 2123, 2136, 2149, 2162, 2175, 2188, 2201, 2214, 2227, 2240, 2253, 3352, 3363, 3374, 3385, 3396, 3407, 3418, 3429, 3440, 3451, 3462, 3473, 3484, 3495, 3506, 3517, 3528, 3539, 3550, 3561, 3572, 3686, 3696, 3718, and 3729.

Within said aspect, it is also envisaged in the context of the present invention to provide an multispecific antigen-binding molecule, wherein the first and second binding domain comprises a VL region selected from the group consisting of SEQ ID Nos: 40, 51, 62, 73, 84, 95, 106, 117, 128, 139, 150, 161, 172, 183, 194, 205, 216, 227, 238, 249, 260, 271, 282, 293, 304, 315, 326, 337, 348, 359, 370, 381, 392, 403, 414, 425, 436, 447, 458, 469, 480, 491, 502, 513, 524, 535, 546, 557, 568, 579, 590, 601, 612, 623, 634, 645, 656, 667, 903, 914, 925, 936, 947, 958, 969, 980, 991, 1002, 1013, 1024, 1035, 1046, 1057, 1068, 1079, 1090, 1101, 1112, 1123, 1134, 1145, 1156, 1167, 1178, 1189, 1200, 1211, 1222, 1233, 1244, 1255, 1266, 1277, 1288, 1299, 1310, 1321, 1332, 1343, 1354, 1365, 1376, 1387, 1398, 1409, 1420, 1431, 1496, 1507, 1518, 1529, 1540, 1551, 1562, 1573, 1584, 1595, 1606, 1617, 1628, 1639, 1650, 1661, 1834, 1847, 1860, 1873, 1886, 1899, 1912, 1929, 1942, 1955, 1968, 1981, 1994, 2007, 2020, 2033, 2046, 2059, 2072, 2085, 2098, 2111, 2124, 2137, 2150, 2163, 2176, 2189, 2202, 2215, 2228, 2241, 2254, 3353, 3364, 3375, 3386, 3397, 3408, 3419, 3430, 3441, 3452, 3463, 3474, 3485, 3496, 3507, 3518, 3529, 3540, 3551, 3562, 3573, 3685, 3697, 3719, and 3730.

Within said aspect, it is also envisaged in the context of the present invention to provide an multispecific antigen-binding molecule, wherein the first and second binding domain comprises a scFv sequence selected from the group consisting of SEQ ID Nos: 41, 52, 63, 74, 85, 96, 107, 118, 129, 140, 151, 162, 173, 184, 195, 206, 217, 228, 239, 250, 261, 272, 283, 294, 305, 316, 327, 338, 349, 360, 371, 382, 393, 404, 415, 426, 437, 448, 459, 470, 481, 492, 503, 514, 525, 536, 547, 558, 569, 580, 591, 602, 613, 624, 635, 646, 657, 668, 671, 674, 677, 680, 683, 686, 689, 692, 695, 698, 701, 704, 707, 710, 713, 716, 719, 722, 725, 728, 731, 734, 737, 740, 743, 746, 749, 752, 755, 758, 761, 764, 767, 770, 773, 776, 779, 782, 785, 788, 791, 794, 797, 800, 803, 806, 809, 812, 815, 818, 821, 824, 827, 830, 833, 836, 839, 842, 845, 848, 851, 854, 857, 860, 863, 866, 869, 872, 874, 876, 878, 880, 882, 884, 886, 888, 890, 892, 894, 904, 915, 926, 937, 948, 959, 970, 981, 992, 1003, 1014, 1025, 1036, 1047, 1058, 1069, 1080, 1091, 1102, 1113, 1124, 1135, 1146, 1157, 1168, 1179, 1190, 1201, 1212, 1223, 1234, 1245, 1256, 1267, 1278, 1289, 1300, 1311, 1322, 1333, 1344, 1355, 1366, 1377, 1388, 1399, 1410, 1421, 1432, 1435, 1438, 1441, 1444, 1447, 1450, 1453, 1456, 1459, 1462, 1465, 1468, 1471, 1474, 1477, 1480, 1483, 1486, 1497, 1508, 1519, 1530, 1541, 1552, 1563, 1574, 1585, 1596, 1607, 1618, 1629, 1640, 1651, 1662, 1665, 1668, 1671, 1674, 1677, 1680, 1683, 1686, 1689, 1692, 1695, 1698, 1701, 1704, 1707, 1710, 1713, 1716, 1719, 1722, 1725, 1728, 1731, 1734, 1737, 1740, 1743, 1746, 1749, 1752, 1755, 1758, 1761, 1764, 1767, 1770, 1773, 1776, 1779, 1782, 1785, 1788, 1791, 1794, 1797, 1800, 1803, 1806, 1809, 1812, 1815, 1818, 1821, 1824, 1835, 1848, 1861, 1874, 1887, 1900, 1913, 1930, 1943, 1956, 1969, 1982, 1995, 2008, 2021, 2034, 2047, 2060, 2073, 2086, 2099, 2112, 2125, 2138, 2151, 2164, 2177, 2190, 2203, 2216, 2229, 2242, 2255, 2264, 2265, 2274, 2275, 2284, 2285, 2294, 2295, 2304, 2305, 2314, 2315, 2324, 2325, 2334, 2335, 2344, 2345, 2354, 2355, 2364, 2365, 2374, 2375, 2384, 2385, 2394, 2395, 2404, 2405, 2414, 2415, 2424, 2425, 2434, 2435, 2444, 2445, 2454, 2455, 2464, 2465, 2474, 2475, 2484, 2485, 2494, 2495, 2504, 2505, 2514, 2515, 2524, 2525, 2534, 2535, 2544, 2545, 2554, 2555, 2564, 2565, 2574, 2575, 2584, 2585, 2594, 2595, 2604, 2605, 2614, 2615, 2624, 2625, 2634, 2635, 2644, 2645, 2654, 2655, 2664, 2665, 2674, 2675, 2684, 2685, 2694, 2695, 2704, 2705, 2714, 2715, 2724, 2725, 2734, 2735, 2744, 2745, 2754, 2755, 2764, 2765, 2774, 2775, 2784, 2785, 2794, 2795, 2804, 2805, 2814, 2815, 2824, 2825, 2834, 2835, 2844, 2845, 2854, 2855, 2864, 2865, 2874, 2875, 2884, 2885, 2894, 2895, 2904, 2905, 2914, 2915, 2914, 2925, 2934, 2935, 2944, 2945, 2954, 2955, 2964, 2965, 2974, 2975, 2984, 2985, 2994, 2995, 3004, 3005, 3014, 3015, 3024, 3025, 3034, 3035, 3044, 3045, 3054, 3055, 3064, 3065, 3074, 3075, 3084, 3085, 3094, 3095, 3104, 3105, 3114, 3115, 3124, 3125, 3134, 3135, 3144, 3145, 3154, 3155, 3164, 3165, 3174, 3175, 3184, 3185, 3194, 3195, 3204, 3205, 3214, 3215, 3224, 3225, 3234, 3235, 3244, 3245, 3254, 3255, 3264, 3265, 3274, 3275, 3284, 3285, 3294, 3295, 3304, 3305, 3314, 3315, 3324, 3325, 3334, 3335, 3354, 3365, 3376, 3387, 3398, 3409, 3420, 3431, 3442, 3453, 3464, 3475, 3486, 3497, 3508, 3519, 3530, 3541, 3552, 3563, 3574, 3577, 3580, 3583, 3586, 3589, 3592, 3595, 3598, 3601, 3604, 3607, 3610, 3613, 3616, 3619, 3622, 3625, 3628, 3631, 3634, 3637, 3640, 3643, 3646, 3649, 3652, 3655, 3658, 3661, 3664, 3667, 3670, 3673, 3676, 3687, 3698, 3701, 3706, 3720, 3731, 3734, 3737, 3740, 3749, 3750, preferably 1399 or 1435.

Within said aspect, it is also envisaged in the context of the present invention to provide an multispecific antigen-binding molecule, comprises a first and/or second target binding domain together with a third effector binding domain, the two or three binding domains together having a sequence selected from the group consisting of SEQ ID Nos: 42, 53, 64, 75, 86, 97, 108, 119, 130, 141, 152, 163, 174, 185, 196, 207, 218, 229, 240, 251, 262, 273, 284, 295, 306, 317, 328, 339, 350, 361, 372, 383, 394, 405, 416, 427, 438, 449, 460, 471, 482, 493, 504, 515, 526, 537, 548, 559, 570, 581, 592, 603, 614, 625, 636, 647, 658, 669, 672, 675, 678, 681, 684, 687, 690, 693, 696, 699, 702, 705, 708, 711, 714, 717, 720, 723, 726, 729, 732, 735, 738, 741, 744, 747, 750, 753, 756, 759, 762, 765, 768, 771, 774, 777, 780, 783, 786, 789, 792, 795, 798, 801, 804, 807, 810, 813, 816, 819, 822, 825, 828, 831, 834, 837, 840, 843, 846, 849, 852, 855, 858, 861, 864, 867, 870 873, 875, 877, 879, 881, 883, 885, 887, 889, 891, 893, 895, 905, 916, 927, 938, 949, 960, 971, 982, 993, 1004, 1015, 1026, 1037, 1048, 1059, 1070, 1081, 1092, 1103, 1114, 1125, 1136, 1147, 1158, 1169, 1180, 1191, 1202, 1213, 1224, 1235, 1246, 1257, 1268, 1279, 1290, 1301, 1312, 1323, 1334, 1345, 1356, 1367, 1378, 1389, 1400, 1411, 1422, 1433, 1436, 1439, 1442, 1445, 1448, 1451, 1454, 1457, 1460, 1463, 1466, 1469, 1472, 1475, 1478, 1481, 1484, 1487, 1498, 1509, 1520, 1531, 1542, 1553, 1564, 1575, 1586, 1597, 1608, 1619, 1630, 1641, 1652, 1663, 1666, 1669, 1672, 1675, 1678, 1681, 1684, 1687, 1690, 1693, 1696, 1699, 1702, 1705, 1708, 1711, 1714, 1717, 1720, 1723, 1726, 1729, 1732, 1735, 1738, 1741, 1744, 1747, 1750, 1753, 1756, 1759, 1762, 1765, 1768, 1771, 1774, 1777, 1780, 1783, 1786, 1789, 1792, 1795, 1798, 1801, 1804, 1807, 1810, 1813, 1816, 1819, 1822, 1825, 1836, 1849, 1862, 1875, 1888, 1901, 1914, 1931, 1944, 1957, 1970, 1983, 1996, 2009, 2022, 2035, 2048, 2061, 2074, 2087, 2300, 2113, 2126, 2139, 2152, 2165, 2178, 2191, 2204, 2217, 2230, 2243, 2256, 2260, 2266, 2267, 2276, 2277, 2286, 2287, 2296, 2297, 2306, 2307, 2316, 2317, 2326, 2327, 2336, 2337, 2346, 2347, 2356, 2357, 2366, 2367, 2376, 2377, 2386, 2387, 2396, 2397, 2406, 2407, 2416, 2417, 2426, 2427, 2436, 2437, 2446, 2447, 2456, 2457, 2466, 2467, 2476, 2477, 2486, 2487, 2496, 2497, 2506, 2507, 2516, 2517, 2526, 2527, 2536, 2537, 2546, 2547, 2556, 2557, 2566, 2567, 2576, 2577, 2586, 2587, 2596, 2597, 2606, 2607, 2616, 2617, 2626, 2627, 2636, 2637, 2646, 2647, 2656, 2657, 2666, 2667, 2676, 2677, 2686, 2687, 2696, 2697, 2706, 2707, 2716, 2717, 2726, 2727, 2736, 2737, 2746, 2747, 2756, 2757, 2766, 2767, 2776, 2777, 2786, 2787, 2796, 2797, 2806, 2807, 2816, 2817, 2826, 2827, 2836, 2837, 2846, 2847, 2856, 2857, 2866, 2867, 2876, 2877, 2886, 2887, 2896, 2897, 2906, 2907, 2916, 2917, 2926, 2927, 2936, 2937, 2946, 2947, 2956, 2957, 2966, 2967, 2976, 2977, 2986, 2987, 2996, 2997, 3006, 3007, 3016, 3017, 3026, 3027, 3036, 3037, 3046, 3047, 3056, 3057, 3066, 3067, 3076, 3077, 3086, 3087, 3096, 3097, 3106, 3107, 3116, 3117, 3126, 3127, 3136, 3137, 3146, 3147, 3156, 3157, 3166, 3167, 3176, 3177, 3186, 3187, 3196, 3197, 3206, 3207, 3216, 3217, 3226, 3227, 3236, 3237, 3246, 3247, 3246, 3247, 3256, 3257, 3266, 3267, 3276, 3277, 3286, 3287, 3296, 3297, 3306, 3307, 3316, 3317, 3326, 3327, 3336, 3337, 3355, 3366, 3377, 3388, 3399, 3410, 3421, 3432, 3443, 3454, 3465, 3476, 3487, 3498, 3509, 3520, 3531, 3542, 3553, 3564, 3575, 3578, 3581, 3584, 3587, 3590, 3593, 3596, 3599, 3602, 3605, 3608, 3611, 3614, 3617, 3620, 3623, 3626, 3629, 3632, 3635, 3638, 3641, 3644, 3647, 3650, 3653, 3656, 3659, 3662, 3665, 3668, 3671, 3674, 3677, 3688, 3699, 3702, 3703, 3707, 3721, 3732, 3735, 3738, 3741, 3751, 3752, preferably 1400 or 1436

Within said aspect, it is also envisaged in the context of the present invention to provide an multispecific antigen-binding molecule, wherein the construct comprises a first and second target binding as disclosed herein, a third domain and optionally a fourth domain conferring extended half-life.

Within said aspect, it is also envisaged in the context of the present invention to provide an antigen-binding molecule, wherein the antigen-binding molecule further comprises in addition to (a) to (d) an amino to carboxyl order:

(e) the first polypeptide monomer of the third domain having a polypeptide sequence selected from the group consisting of SEQ ID NOs: 17-24;
(f) a peptide linker having an amino acid sequence selected from the group consisting of SEQ ID NOs: 5, 6, 7 and 8; and
(g) the second polypeptide monomer of the third domain having a polypeptide sequence selected from the group consisting of SEQ ID NOs: 17-24.

Within said aspect, it is also envisaged in the context of the present invention to provide an antigen-binding molecule, having an amino acid sequence selected from the group consisting of SEQ ID NOs: 41, 52, 63, 74, 85, 96, 107, 118, 129, 140, 151, 162, 173, 184, 195, 206, 217, 228, 239, 250, 261, 272, 283, 294, 305, 316, 327, 338, 349, 360, 371, 382, 393, 404, 415, 426, 437, 448, 459, 470, 481, 492, 503, 514, 525, 536, 547, 558, 569, 580, 591, 602, 613, 624, 635, 646, 657, 668, 671, 674, 677, 680, 683, 686, 689, 692, 695, 698, 701, 704, 707, 710, 713, 716, 719, 722, 725, 728, 731, 734, 737, 740, 743, 746, 749, 752, 755, 758, 761, 764, 767, 770, 773, 776, 779, 782, 785, 788, 791, 794, 797, 800, 803, 806, 809, 812, 815, 818, 821, 824, 827, 830, 833, 836, 839, 842, 845, 848, 851, 854, 857, 860, 863, 866, 869, 872, 874, 876, 878, 880, 882, 884, 886, 888, 890, 892, 894, 904, 915, 926, 937, 948, 959, 970, 981, 992, 1003, 1014, 1025, 1036, 1047, 1058, 1069, 1080, 1091, 1102, 1113, 1124, 1135, 1146, 1157, 1168, 1179, 1190, 1201, 1212, 1223, 1234, 1245, 1256, 1267, 1278, 1289, 1300, 1311, 1322, 1333, 1344, 1355, 1366, 1377, 1388, 1399, 1410, 1421, 1432, 1435, 1438, 1441, 1444, 1447, 1450, 1453, 1456, 1459, 1462, 1465, 1468, 1471, 1474, 1477, 1480, 1483, 1486, 1497, 1508, 1519, 1530, 1541, 1552, 1563, 1574, 1585, 1596, 1607, 1618, 1629, 1640, 1651, 1662, 1665, 1668, 1671, 1674, 1677, 1680, 1683, 1686, 1689, 1692, 1695, 1698, 1701, 1704, 1707, 1710, 1713, 1716, 1719, 1722, 1725, 1728, 1731, 1734, 1737, 1740, 1743, 1746, 1749, 1752, 1755, 1758, 1761, 1764, 1767, 1770, 1773, 1776, 1779, 1782, 1785, 1788, 1791, 1794, 1797, 1800, 1803, 1806, 1809, 1812, 1815, 1818, 1821, and 1824, preferably 1399 or 1435, preferably SEQ ID NOs 42, 53, 64, 75, 86, 97, 108, 119, 130, 141, 152, 163, 174, 185, 196, 207, 218, 229, 240, 251, 262, 273, 284, 295, 306, 317, 328, 339, 350, 361, 372, 383, 394, 405, 416, 427, 438, 449, 460, 471, 482, 493, 504, 515, 526, 537, 548, 559, 570, 581, 592, 603, 614, 625, 636, 647, 658, 669, 672, 675, 678, 681, 684, 687, 690, 693, 696, 699, 702, 705, 708, 711, 714, 717, 720, 723, 726, 729, 732, 735, 738, 741, 744, 747, 750, 753, 756, 759, 762, 765, 768, 771, 774, 777, 780, 783, 786, 789, 792, 795, 798, 801, 804, 807, 810, 813, 816, 819, 822, 825, 828, 831, 834, 837, 840, 843, 846, 849, 852, 855, 858, 861, 864, 867, 870 873, 875, 877, 879, 881, 883, 885, 887, 889, 891, 893, 895, 905, 916, 927, 938, 949, 960, 971, 982, 993, 1004, 1015, 1026, 1037, 1048, 1059, 1070, 1081, 1092, 1103, 1114, 1125, 1136, 1147, 1158, 1169, 1180, 1191, 1202, 1213, 1224, 1235, 1246, 1257, 1268, 1279, 1290, 1301, 1312, 1323, 1334, 1345, 1356, 1367, 1378, 1389, 1400, 1411, 1422, 1433, 1436, 1439, 1442, 1445, 1448, 1451, 1454, 1457, 1460, 1463, 1466, 1469, 1472, 1475, 1478, 1481, 1484, 1487, 1498, 1509, 1520, 1531, 1542, 1553, 1564, 1575, 1586, 1597, 1608, 1619, 1630, 1641, 1652, 1663, 1666, 1669, 1672, 1675, 1678, 1681, 1684, 1687, 1690, 1693, 1696, 1699, 1702, 1705, 1708, 1711, 1714, 1717, 1720, 1723, 1726, 1729, 1732, 1735, 1738, 1741, 1744, 1747, 1750, 1753, 1756, 1759, 1762, 1765, 1768, 1771, 1774, 1777, 1780, 1783, 1786, 1789, 1792, 1795, 1798, 1801, 1804, 1807, 1810, 1813, 1816, 1819, 1822, and 1825, preferably 1400 or 1436, more preferably 43, 54, 65, 76, 87, 98, 109, 120, 131, 142, 153, 164, 175, 186, 197, 208, 219, 230, 241, 252, 263, 274, 285, 296, 307, 318, 329, 340, 351, 362, 373, 384, 395, 406, 417, 428, 439, 450, 461, 472, 483, 494, 505, 516, 527, 538, 549, 560, 571, 582, 593, 604, 615, 626, 637, 648, 659, 670, 673, 676, 679, 682, 685, 688, 691, 694, 697, 700, 703, 706, 709, 712, 715, 718, 721, 724, 727, 730, 733, 736, 739, 742, 745, 748, 751, 754, 757, 760, 763, 766, 769, 772, 775, 778, 781, 784, 787, 790, 793, 796, 799, 802, 805, 808, 811, 814, 817, 820, 823, 826, 829, 832, 835, 838, 841, 844, 847, 850, 853, 856, 859, 862, 865, 868, 871, 906, 917, 928, 939, 950, 961, 972, 983, 994, 1005, 1016, 1027, 1038, 1049, 1060, 1071, 1082, 1093, 1104, 1115, 1126, 1137, 1148, 1159, 1170, 1181, 1192, 1203, 1214, 1225, 1236, 1247, 1258, 1269, 1280, 1291, 1302, 1313, 1324, 1335, 1346, 1357, 1368, 1379, 1390, 1401, 1412, 1423, 1434, 1437, 1440, 1443, 1446, 1449, 1452, 1455, 1458, 1461, 1464, 1467, 1470, 1473, 1476, 1479, 1482, 1485, 1488, 1499, 1510, 1521, 1532, 1543, 1554, 1565, 1576, 1587, 1598, 1609, 1620, 1631, 1642, 1653, 1664, 1667, 1670, 1673, 1676, 1679, 1682, 1685, 1688, 1691, 1694, 1697, 1700, 1703, 1706, 1709, 1712, 1715, 1718, 1721, 1724, 1727, 1730, 1733, 1736, 1739, 1742, 1745, 1748, 1751, 1754, 1757, 1760, 1763, 1766, 1769, 1772, 1775, 1778, 1781, 1784, 1787, 1790, 1793, 1796, 1799, 1802, 1805, 1808, 1811, 1814, 1817, 1820, 1823, 1826, 1838, 1851, 1864, 1877, 1890, 1903, 1916, 1933, 1946, 1959, 1972, 1985, 1998, 2011, 2024, 2037, 2050, 2063, 2076, 2089, 2102, 2115, 2128, 2141, 2154, 2167, 2180, 2194, 2206, 2219, 2232, 2245, 2258, 2262, 2270, 2271, 2280, 2281, 2290, 2291, 2300, 2301, 2310, 2311, 2320, 2321, 2330, 2331, 2340, 2341, 2350, 2351, 2360, 2361, 2370, 2371, 2380, 2381, 2390, 2391, 2400, 2401, 2410, 2411, 2420, 2421, 2430, 2431, 2440, 2441, 2450, 2451, 2460, 2461, 2470, 2471, 2480, 2481, 2490, 2491, 2500, 2501, 2510, 2511, 2520, 2521, 2530, 2531, 2540, 2541, 2550, 2551, 2560, 2561, 2570, 2571, 2580, 2581, 2590, 2591, 2600, 2601, 2610, 2611, 2620, 2621, 2630, 2631, 2640, 2641, 2650, 2651, 2660, 2661, 2670, 2671, 2680, 2681, 2690, 2691, 2700, 2701, 2710, 2711, 2720, 2721, 2730, 2731, 2740, 2741, 2750, 2751, 2760, 2761, 2770, 2771, 2780, 2781, 2790, 2791, 2800, 2801, 2810, 2811, 2820, 2821, 2830, 2831, 2840, 2841, 2850, 2851, 2860, 2861, 2870, 2871, 2880, 2881, 2890, 2891, 2900, 2901, 2910, 2911, 2920, 2921, 2930, 2931, 2940, 2941, 2950, 2951, 2960, 2961, 2970, 2971, 2980, 2981, 2990, 2991, 3000, 3001, 3010, 3011, 3020, 3021, 3030, 3031, 3040, 3041, 3050, 3051, 3060, 3061, 3070, 3071, 3080, 3081, 3090, 3091, 3100, 3101, 3110, 3111, 3120, 3121, 3130, 3131, 3140, 3141, 3150, 3151, 3160, 3161, 3170, 3171, 3180, 3181, 3190, 3191, 3200, 3201, 3210, 3211, 3220, 3221, 3231, 3240, 3241, 3250, 3251, 3260, 3261, 3270, 3271, 3280, 3281, 3290, 3291, 3300, 3301, 3310, 3311, 3320, 3321, 3330, 3331, 3340, 3341, 3344, 3345, 3356, 3367, 3378, 3389, 3400, 3411, 3422, 3433, 3444, 3455, 3466, 3477, 3488, 3499, 3510, 3521, 3532, 3543, 3554, 3565, 3576, 3579, 382, 3585, 3588, 3591, 3594, 3597, 3600, 3603, 3606, 3609, 3612, 3615, 3618, 3621, 3624, 3627, 3630, 3633, 3636, 3639, 3642, 3645, 3648, 3651, 3654, 3657, 3660, 3663, 3666, 3669, 3672, 3675, 3678, 3689, 3700, 3704, 3705, 3708, 3709, 3710, 3711, 3722, 3733, 3736, 3739, 3744, 3747, 3748, 3756, 3757, 3761, and 3762 preferably 1401 or 1437.

Within said aspect, it is also envisaged in the context of the present invention to provide an antigen-binding molecule which has a stability-optimized CD3 binder, the molecule having an amino acid sequence selected from the group consisting of 1839, 1852, 1865, 1878, 1891, 1904, 1917, 1919, 1921, 1934, 1947, 1960, 1973, 1986, 1999, 2012, 2025, 2038, 2051, 2064, 2077, 2090, 2103, 2116, 2029, 2142, 2155, 2168, 2181, 2193, 2207, 2220, 2233, 2246, 2259, 2263, 2272, 2273, 2282, 2283, 2292, 2293, 2302, 2303, 2312, 2313, 2322, 2323, 2332, 2333, 2342, 2343, 2352, 2353, 2362, 2363, 2372, 2373, 2382, 2383, 2392, 2393, 2402, 2403, 2412, 2413, 2422, 2423, 2432, 3433, 2442, 2443, 2452, 2453, 2462, 2463, 2472, 2473, 2482, 2483, 2492, 2493, 2502, 2503, 2512, 2513, 2522, 2523, 2532, 2533, 2542, 2543, 2552, 2553, 2562, 2563, 2572, 2573, 2582, 2583, 2592, 2593, 2602, 2603, 2612, 2613, 2622, 2623, 2632, 2633, 2642, 2643, 2652, 2653, 2662, 2663, 2672, 2673, 2682, 2683, 2692, 2693, 2702, 2703, 2712, 2713, 2722, 2723, 2732, 2733, 2742, 2743, 2752, 2753, 2762, 2763, 2772, 2773, 2782, 2783, 2792, 2793, 2802, 2803, 2812, 2813, 2822, 2823, 2832, 2833, 2842, 2843, 2852, 2853, 2862, 2863, 2872, 2873, 2882, 2883, 2892, 2893, 2902, 2903, 2912, 2913, 2922, 2923, 2932, 2933, 2942, 2943, 2952, 2953, 2962, 2963, 2972, 2973, 2982, 2983, 2992, 2993, 3002, 3003, 3012, 3013, 3022, 3023, 3032, 3033, 3042, 3043, 3052, 3053, 3062, 3063, 3072, 3073, 3082, 3083, 3092, 3093, 3112, 3113, 3122, 3123, 3132, 3133, 3142, 3143, 3152, 3153, 3162, 3163, 3172, 3173, 3182, 3183, 3192, 3193, 3202, 3203, 3212, 3213, 3222, 3223, 3232, 3233, 3242, 3243, 3252, 3253, 3262, 3263, 3272, 3273, 3282, 3283, 3292, 3293, 3302, 3303, 3312, 3313, 3322, 3323, 3332, 3333, 3342, 3343, 3745, 3746, 3758, 3759, and 3760.

In a second aspect, it is further envisaged in the context of the present invention to provide a polynucleotide encoding an antigen-binding molecule of the present invention.

In a third aspect, it is also envisaged in the context of the present invention to provide a vector comprising a polynucleotide of the present invention.

In a fourth aspect, it is further envisaged in the context of the present invention to provide a host cell transformed or transfected with the polynucleotide or with the vector of the present invention.

In a fifth aspect, it is also envisaged in the context of the present invention to provide a process for the production of an antigen-binding molecule of the present invention, said process comprising culturing a host cell of the present invention under conditions allowing the expression of the antigen-binding molecule and recovering the produced antigen-binding molecule from the culture.

In a sixth aspect, it is further envisaged in the context of the present invention to provide a pharmaceutical composition comprising an antigen-binding molecule of the present invention or produced according to the process of the present invention.

Within said aspect, is also envisaged in the context of the present invention that the pharmaceutical composition is stable for at least four weeks at about −20° C.

It is further envisaged in the context of the present invention to provide the antigen-binding molecule of the present invention, or produced according to the process of the present invention, for use in the prevention, treatment or amelioration of a disease selected from a proliferative disease, a tumorous disease, cancer or an immunological disorder.

Within said aspect, it is also envisaged in the context of the present invention that the disease preferably is multiple myeloma (MM), acute myeloid leukemia (AML), Non-Hodgkin lymphoma (NHL), Non-small-cell lung carcinoma (NSCLC) and Colorectal cancer (CRC), wherein preferably a CS1xBCMA or BCMAxCS1 multispecific antigen-binding molecule is for use in the treatment of multiple myeloma, wherein preferably a FLT3xCD123 or CD123xFLT3 multispecific antigen-binding molecule is for use in the treatment of acute myeloid leukemia, and wherein preferably a CD20xCD22 or CD22xCD20 multispecific antigen-binding molecule is for use in the treatment of Non-Hodgkin lymphoma.

In a seventh aspect, it is further envisaged in the context of the present invention to provide a method for the treatment or amelioration of a proliferative disease, a tumorous disease, cancer, or an immunological disorder, comprising the step of administering to a subject in need thereof the antigen-binding molecule of the present invention, or produced according to the process of the present invention, wherein the disease preferably is multiple myeloma, acute myeloid leukemia, Non-Hodgkin lymphoma, Non-small-cell lung carcinoma and/or Colorectal cancer, wherein preferably a CS1xBCMA or BCMAxCS1 multispecific antigen-binding molecule is for the treatment of multiple myeloma, wherein preferably a FLT3xCD123 or CD123xFLT3 multispecific antigen-binding molecule is for the treatment of acute myeloid leukemia, and wherein preferably a CD20xCD22 or CD22xCD20 multispecific antigen-binding molecule is for the treatment of Non-Hodgkin lymphoma.

In an eighth aspect, it is also envisaged in the context of the present invention to provide a kit comprising an antigen-binding molecule of the present invention, or produced according to the process of the present invention, a polynucleotide of the present invention, a vector of the present invention, and/or a host cell of the present invention.

### DESCRIPTION OF THE FIGURES

FIG. 1 shows the general setup of multispecific antigen-binding molecules of the present invention.

FIG. 2 shows a schematic model overview why a multispecific antigen-binding molecule according to the present invention with a short (6 aa) peptidic linker between the first and second binding domain would not be expected to be able to bind to the TAA2 (A), while it would with a longer peptidic linker of 30 aa (B). The third binding domain and the optional fourth domain are not shown for readability.

FIG. 3 shows an overview of three different modeled confirmations of the second binding domain with respect to the first binding domain of example multispecific antigen-binding molecule MSLNxEpCAM HLE BiTE® antigen-binding molecule (A), conformation 2 allowing a maximum space for the TAA2 of 10 to 30 Å (B), conformation 1 allowing a maximum space for the TAA2 of 3 to 8 Å (C), and conformation 3 allowing no space for the TAA2, i.e. provoking a steric clash between the binding domains (D).

FIG. 4: FIG. 4 shows the modeled space between the first and the second binding domain of example multispecific antigen-binding molecule MSLNxEpCAM HLE BiTE® antigen-binding molecule depending on the linker length, wherein a flexible (SGn)x linker, with n being 2 to 4 and x being 3 to 6, has been examined. The maximum available space is 39-50 Å for a 12 aa long exemplary linker (A), 54-60 Å for a 18 aa long exemplary linker (B), and 84-94 Å for a 30 aa long exemplary linker (C). For a CD20xCD22 HLE BiTE® molecule, the available space is modeled with a 5 aa long linker (D), 30 aa long linker (E) and a 50 aa long linker (F), as well as for a CS1xBCMA HLE BiTE® molecule, the available space is modeled with a 5 aa long linker (G), 30 aa long linker (H) and a 50 aa long linker (1).

FIG. 5: FIG. 5 shows binding experiments using electrically switchable nanolever technology. Association and dissociation of exemplary MSLNxEpCAM HLE BiTE® antigen-binding molecule measured in three concentrations (12.5 nM, 25 nM and 50 nM) is shown (A, B, C, D). Tested biochip was coated with a 1:1 mix of antigen EpCAM (A, B) and antigen MSLN (C, D). Quenching of green fluorophore is shown, respectively binding to antigen EpCAM (A, B). Quenching of red fluorophore is shown, respectively binding to antigen MSLN (C, D). Binding experiments using switchSENSE® (Planegg, Germany) technology are further shown in E, F, G, H, I J, K, L M, N, O and P. Association and dissociation of the dual targeting CLL1xFLT3 HLE BiTE® antigen-binding molecule in three concentrations (12.5 nM, 25 nM and 50 nM) is shown. Tested biochip was coated with either antigen CLL1 (E, F), antigen FLT3 (G, H) or a 1:1 mix of both antigens CLL1 and FLT3 (I, J). Association and dissociation of the dual targeting CS1xBCMA HLE BiTE® antigen-binding molecule in three concentrations (12.5 nM, 25 nM and 50 nM) is shown. Tested biochip was coated with either antigen CS1 (K, L), antigen BCMA (M, N) or a 1:1 mix of both antigens CS1 and BCMA (O, P).

FIG. 6: FIG. 6 shows CS1 and BCMA surface expression in human multiple myeloma cell lines and additional human blood cancer cell lines. The antibody clone 162.1 was used to detect CS1 (•) while the antibody clone 19F2 was used to detect BCMA ( [symbol] ).

FIG. 7: FIG. 7 shows re-directed T cell lysis (A), activation of T cells (B), and induction of cytokines (C) by multitargeting (dual) CS1 x BCMA BiTE® antigen-binding molecule using ARH-77 target cells.

FIG. 8: FIG. 8 shows re-directed T cell lysis (A), activation of T cells (B), and induction of cytokines (C, D) using T cell sub-populations by multitargeting (dual) CS1 x BCMA BiTE® antigen-binding molecule using MM.1R target cells.

FIG. 9: FIG. 9 shows re-directed T cell lysis by multitargeting CS1 x BCMA (dual) BiTE® antigen-binding molecule, CS1 (mono) BiTE® antigen-binding molecule, and BCMA (mono) BiTE® antigen-binding molecule using ARH-77 (A), MM.1R (B), OPM-2 (C), and U266B1 (D) target cells.

FIG. 10: FIG. 10 shows re-directed T cell lysis by the multitargeting CS1 x BCMA Dual BiTE® antigen-binding molecule, CS1 Mono BiTE® antigen-binding molecule, and BCMA Mono BiTE® antigen-binding molecule in the presence of soluble CS1 and/or soluble BCMA at different concentrations from 0 ng/ml of each (A) up to 80 ng/ml soluble CS1 and 250 ng/ml soluble BCMA (G) using U266B1 target cells.

FIG. 11: FIG. 11 shows re-directed T cell lysis by the multispecific CS1 x BCMA Dual BiTE®, CS1 Mono BiTE® antigen-binding molecule, and BCMA Mono BiTE® antigen-binding molecule in the presence of soluble CS1 or soluble BCMA at different concentrations from 0 ng/ml of each (A) up to 250 ng/ml soluble BCMA (I) using OPM-2 target cells

FIG. 12: FIG. 12 shows re-directed T cell lysis by the multispecific CS1 x BCMA dual BiTE® antigen-binding molecule, CS1 Mono BiTE® antigen-binding molecule, and BCMA Mono BiTE® antigen-binding molecule in the presence of soluble BCMA using U266B1 (A to C), NCI-H929 (D to F) and OPM-2 (G to I) target cell lines.

FIG. 13: FIG. 13 shows Re-directed T cell lysis using T cell sub-populations Pan T cells (A), CD4+ T cells (B) and CD8⁺ T cells (C) by the CS1 x BCMA Dual BiTE® antigen-binding molecule, CS1 Mono BiTE® antigen-binding molecule, and BCMA Mono BiTE® antigen-binding molecule using U266B1 target cells.

FIG. 14: FIG. 14 shows re-directed T cell lysis using T cell sub-populations by the CS1 x BCMA Dual BiTE® antigen-binding molecule using U266B1 target cells.

FIG. 15: FIG. 15 shows J chain mRNA levels plotted against BiTE® concentration of CS1 mono BiTE® antigen-binding molecule (CS1 HLE BiTE®) vs. BCMA mono BiTE® antigen-binding molecule vs. multitargeting CS1xBCMA BiTE® antigen-binding molecule (dBiTE).

FIG. 16: FIG. 16 (A to C) shows target cell lysis by two representative CD20-CD22 T-cell engager molecules compared to equimolar mixtures of CD20 and CD22 T-cell engager molecules on Ramos wt cells.

FIG. 17: BCMA-CS1 T-cell molecule 1 with 6 amino acid linker showed comparable EC50 values [pM] on OPM-2 cells as engager molecules 2 and 3 with longer linker variants (FIG. 17A). EpCAM-MSLN T-cell engager molecules 2 and 3 with longer linker variants showed comparable EC50 values [pM] on HCT-116 cells as EpCAM-MSLN T-cell engager molecule 1 with original 6 amino acid linker (FIG. 17B). CD123-FLT3 T-cell engager molecules 2 and 3 with longer linker variants showed comparable EC50 values [pM] on OPM-2 cells as CD123-FLT3 T-cell engager molecule 1 with original 6 amino acid linker (FIG. 17C).

FIG. 18: The tested CD22 T-cell engager molecule 1 showed comparable EC50 values [pM] on Raji cells (FIG. 18A) as CD20-CD22 T-cell engager molecule 1 on Raji CRISPR CD20 cells (FIG. 18B).

FIG. 19: FIG. 19 depicts alignments of CD20xCLL1 protein sequences of human and mouse with epitope sections. The extracellular loop 1 (ECL1) of CD20 protein was designated E1, extracellular loop 2 (ECL2) was designated E2. For more refined epitope mapping, the extracellular loop 1 (ECL1) was further divided into ElA and E1B subparts and extracellular loop 2 (ECL2) was further divided into the subparts E2A, E2B, E2C or E2D.

FIG. 20: Flow Cytometry Binding Analysis of known CD20 Antibodies (A) and T Cell Engager Molecules as disclosed herein (B) on transfected CHO cells Expressing Full-length Human or Mouse CD20 Protein Constructs or Human/Mouse Chimeric CD20 Protein Constructs.

FIG. 21: Sequence Alignment of Human and Mouse CD22. Sequence above: human CD22, sequence below: mouse CD22. Sequence alignment of the CD22 protein shows each mouse sequence part (V+C2-1, C2-1+C2-2, C2-2+C2-3, C2-3+C2-4, C2-4+C2-5, C2-5+C2-6) that was replaced with the corresponding human sequence and which amino acids differ between the two species. To guarantee structure integrity of the transition area between domains two adjacent domains of mouse CD22 protein were swapped to the corresponding sequence of human CD22 protein. To get a single domain specific result epitope clustering constructs were designed to overlap each other. The swapped sequence regions comprise two CD22 domains, the amino acid stretch between the swapped domains and the amino acid stretch between the second swapped domain and the following CD22 domain.

FIG. 22: Flow Cytometry Binding Analysis of CD22 Antibodies and T Cell Engager Molecules on HEK Cell Membrane Fragments: Truncated CD22 proteins—controls, i.e. known antibodies (A), Mouse/human chimeric CD22 proteins—controls, i.e. known antibodies (B), Truncated CD22 proteins—CD22 T cell engager molecules disclosed herein (C).

FIG. 23: FACS based cytotoxicity assays testing CD20xCD22 antigen-binding molecules U4U, Z3L, G3P, Y3N, B5K and C8V as disclosed herein on target cells CHO ff/Luc pCMV/hu orl CD22 v1 pEFDHFR with Effector cells unstim. PBMC #263 (E:T-Ratio 50.000:5.000 S100 μl) in RPMI plus+10% FCS at a starting concentration of 160 nM and a dilution of 1:6 on F-bottom plate (A), on target cells CHO huCD20 pEFDHFR/ffLuc pCMV with Effector cells unstim. PBMC #263 (E:T-Ratio 50.000:5.000 S100 μl) in RPMI plus+10% FCS at a starting concentration of 160 nM and a dilution of 1:6 on F-bottom plate (B), and LUC based cytotoxicity assays testing CD20xCD22 antigen-binding molecule Y3N and CD20 antigen-binding molecules T9J and S3 as disclosed herein on target cells huCHO CD20+ Effector cells unstim. PBMC #773 (E:T-Ratio 25.000:2.500 S50 μl) in RPMI plus+10% FCS at a starting concentration of 160 nM and a dilution of 1:6 on 384-well F-bottom plate (C), and on target cells huCHO CD22+ Effector cells unstim. PBMC #773 (E:T-Ratio 25.000:2.500 S50 μl) in RPMI plus+10% FCS at a starting concentration of 160 nM and a dilution of 1:6 on 384-well F-bottom plate (D).

FIG. 24: Three dual targeting CLL1xFLT3 antigen binding molecules with three different arrangements of domains have been tested for cytotoxic activity on huCHO FLT3 positive cells, huCHO CLL1 positive cells and double positive cells (DT): format 1 1CL1 9-G4 CC x I2C0 x scFc x FL 4-E9 CC (A), format 2 CL1 9-G4 CC x FL 4-E9 CC x I2C0 x ScFc (B), and format 3 CL1 9-G4 CC x ScFc x 4-E9 CC x I2C0 (C).

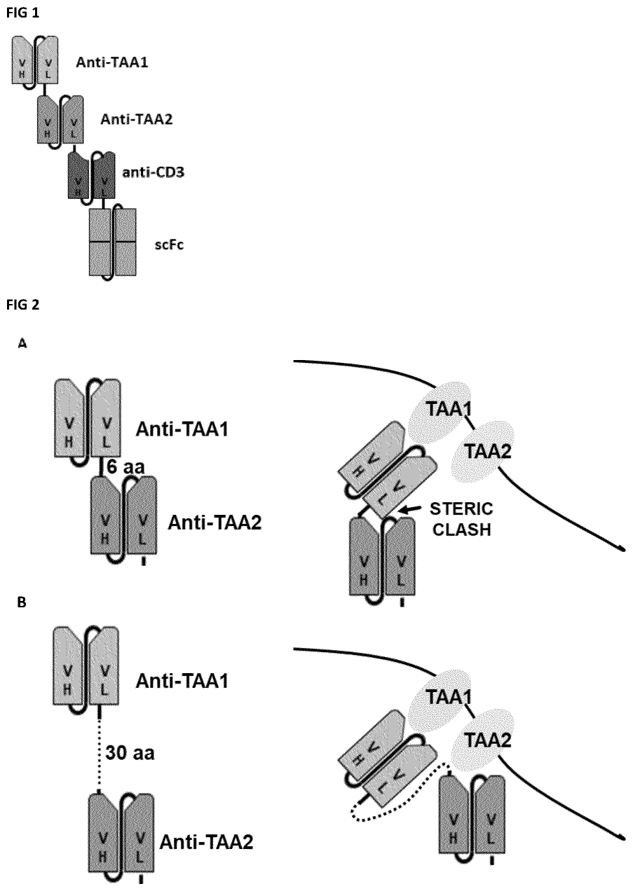

### DETAILED DESCRIPTION

In the context of the present invention, a multitargeting antigen-binding molecule is provided comprising at least three binding domains, wherein the first and second binding domain in amino to carboxyl orientation are capable to preferably target two target cell surface antigens associated with a malignancy simultaneously, wherein the third binding domain binds to an extracellular epitope of the human and/or the Macaca CD3ε chain on an effector cell which is a T cell.

It is a surprising finding in the context of the present invention that the T-cell engaging multispecific antigen-binding molecules according to the present invention are preferably suited to target two (different) antigens on one target cells, such as cancer cells, and in contrast, do less target non-cancer cells. By being capable to address two target antigens at the same time, (a) the likeliness of targeting a target cell such as a cancer cell is greatly increased once such target cell has undergone antigen loss and, thus, is prone to tumor escape from effective anti-tumor therapy because one valid antigen to target remains on the cell which has undergone antigen escape, and (b) the likeliness of targeting a target cell associated with a disease instead of a physiologic cell is greatly increased when two TAAs are chosen which are typically associated with a target cell associated with a disease instead of a physiologic cell. In this regard, multitargeting antigen-binding molecules are envisaged herein, which do not only prevent antigen escape e.g. in a tumor setting, but so furthermore widen the therapeutic window by addressing cells with a pattern of, e.g., two antigens which re typically associated with a particular disease. Thereby, physiologic tissue whose cells express only one of the two targets is not addressed by the instant dual targeting antigen-binding molecules. In particular, a selectivity gap can be achieved by dual targeting molecules, e.g. of formats as described herein, which have a bispecific entity comprising a target binding domain (or binder, as synonymously used throughout this disclosure) and a CD3 binder, a further target binder and optionally a half-life extending domain such as a scFc domain. Dual targeting antigen-binding molecules as described herein typically feature EC50 values below 100 pM, preferably below 50 pM, more preferably below 30 pM and even more preferably about 10 pM or below on cells positive for both targets while such dual targeting molecules typically show significantly higher EC50 values (e.g. at least 50 pM, 100 pM, 250 pM or even 500 pM and higher) when employed with mono-targeting cells. This finding suggests that multitargeting molecules of the present invention do have selectivity gaps in terms of activity of at least factor 10, preferably at least factor 20 or even 30, which can beneficially be used to specifically address pathogenic target cells which express both targets and which can be bound at the same time by said molecules in order to trigger T-cell mediated cytotoxicity. Off-target toxicities and related side effects can thereby be reduced and a safer therapy can be provided based on the instantly described concept. Hence, a T-cell engaging multitargeting antigen-binding molecules according to the present invention, which is typically singe-chained, both provides improved efficacy and safety with regard to existing bispecific antibodies or antigen-binding molecules which are T-cell engaging. Said advantageous properties are preferably achieved by the fact that the first and the second binding domain of the multitargeting antigen-binding molecule are capable to independently from each other to maintain their bioactivity, i.e. to bind their respective targets without being sterically hindered by the respective other binding domain and/or the target to which the respective other target binder has bound. The preserved bioactivity is preferably achieved by (a) the VH-VL setup in amino to carboxyl orientation of both binding domains and/or (b) the careful selection of the linker which links the first and the second binding domain. Said linker needs to have a length with ensures both bioactivity of both binding domains and sufficient (chemical) stability of the construct. Surprisingly relatively short peptide linkers of about 5 to 24, preferably 5 to 18, more preferably 6 or 12 amino acids in length fulfil both requirements. Preferably, such linkers are rich in small or hydrophilic amino acids, such as Gly and Ser, because such composition preferably provides flexibility. In consequence, such flexibility preferably allows for interaction of the respective binding domain independently of the other binding domain of the multitargeting antigen-binding molecule according to the present invention. At the same time, it is surprising that even such short preferably flexible peptide linkers typically provide for sufficient spatial separation between the first and the second binding domain so that both domains retain their bioactivity which is required to have a therapeutically useful molecule in the context of the present invention. An additional advantage of such short linkers as disclosed in the context of the present invention is that interchain mispairings re preferably prevented in comparison to longer linkers.

The above-specified finding underlying the present invention is surprising in view of the teaching of the prior art. For example, Liu et al. showed that the longer the inter-peptide linkers were, the better the preservation of the independent folding and biological activities of the two molecules (Liu Z G, Lin J B, Du W, et al. Anti-proteolysis study of recombinant IIn-UK fusion protein in CHO cell. Prog Biochem Biophys 2005; 32:544-50). Linkers between binding domains, preferably scFv binding domains, that are too short negatively affect protein folding by spatial occupancy, and those that are too long enhance the antigenicity of the scFv antibody and also affect the functionality and activity of scFv antibodies. Xu et al. teach that sufficient length and certain sequence characteristics are the key factors that provide the two half-molecules with sufficient free space to fulfill their functions, and avoiding the formation of the a-helix and b-sheet is important for stability (Xue F, Gu Z, Feng J A. LINKER: a web server to generate peptide sequences with extended conformation. Nucleic Acids Res 2004; 32:W562-5). Hence, the skilled person aiming to maintain distance between binding domains would have contemplated to employ rigid linkers which typically feature a helical structure or are rich in proline. However, also the length of the rigid linkers has a major impact on protein bioactivity. McCormick et al examined rigid peptide linkers (Ala-Pro)n (10-34 aa) which were applied in an interferon-γ-gp120 fusion protein (McCormick A, Thomas M, Heath A. Immunization with an interferon-gamma-gp120 fusion protein induces enhanced immune responses to human immunodeficiency virus gp120. J Infect Dis. 2001; 184:1423-1430). With a short 10-aa linker, the fusion protein possessed a relatively low biological activity of interferon-γ. By increasing the linker length, the bioactivity of the fusion protein was gradually improved, peaking at 88% activity of free interferon-γ with the longest 34-residue linker. Even more, in some cases even with the insertion of flexible or rigid linkers, the impaired bioactivity can still not be overcome due to steric hindrance between domains (Bai Y, Ann D K, Shen W C. Recombinant granulocyte colony-stimulating factor-transferrin fusion protein as an oral myelopoietic agent. Proc Natl Acad Sci USA. 2005; 102:7292-7296).

In view of the obstacles know in the art, the skilled person would have been prompted to avoid short flexible or even rigid linkers and would turn to longer rigid lingers, wherein “long” could be understood from the art as about 30 amino acids, preferably comprising proline. Based on this information, the skilled person would preferably model the first and the second binding domain linked by a peptide linker to confirm what linker length to take and which to avoid using state of the art modeling technology. Provided the linker is a flexible linker rich in Ger and Ser, a linker length of 30 amino acids would typically lead to a rather large space between the first and the second binding domain, typically of at least 70 Å, more typically of at least 80 Å, which the skilled person would consider safe in size to accommodate the second target cell surface antigen (e.g. TAA2) to facilitate binding by the second binding domain of the multitargeting antigen-binding molecule. It is important to note in the context of the present invention that while the first binding domain, i.e. the N-terminal binding domain, is comparably easy to access as it has only one adjacent binding domain which potentially causes steric hindrance when binding to the target, the second binding domain is connected to the first binding domain in N-direction

Typically, when a SGGGGS linker is modeled between the two target binding domains which are scFvs (e.g. MSLNxEpCAM), when a (GGGGS)3 linker between the VH and VL within the binding domains, respectively, when the first binding domain, e.g. an anti-MSLN binding domain, is fixed, and when three likely expected conformations are applied where the linker swings in different orthogonal (linker conformation 1, 2 and 3, respectively), then in case of linker position 3, a complete clash is observed, while in positions 1 and 2, no clash is observed. However, the space is typically still not enough to accommodate the TAA2 EpCAM based on where the CDRs are preferably located in the second binding domain of the multitargeting antigen-binding molecule according to the present invention. Hence, this result strongly indicates the need of a longer linker between the two target binding domains. If the skilled person used the size of target EpCAM as guide, one would predict a better linker to be one that has preferably at least about 30 residues, less preferred at least 20 residues (i.e. 70 A preferred distance divided by 3.8 per aa). Accordingly, lack of space renders a short linker solution such as a SGGGS linker and short multiplicities thereof (e.g. S(G4S)2 and S(G4S)2 between the two target binding domains according to the present invention a non-preferred and therefore non-obvious choice for this setup of target binders in a multitargeting antigen-binding molecule, in particular a dual targeting BiTE® molecule. The same applies to a linker of 12 aa which typically offers a maximum available space as small as about 35 Å which, depending on the circumstances, can be up to about 50 Å which would not safely accommodate typical target to be bound which is at least about 45, 50, 55, 60, 65, 70, 75, 80 or 85 Å in size. Also, an 18 aa long linker (e.g. SGGGGSGGGGSGGGGSGG) with a maximum available space between binding domains in a setup as described herein of not more than 60 Å, typically not more than 55 Å, for example, 54 to 60 Å, would likely not allow binding to the second TAA2 of an exemplary size of 45 to 70 Å. In contrast, a 30 aa long linker would typically offer 84 to 94 Å of maximum space, thus safely allowing the target binder to bind its exemplified target EpCAM of about 45 to 70 Å. Thus, the skilled person would have chosen a linker length at least greater than 18 aa to ensure binding of the second TAA2, such as in a MSLNxEpCAM HLE dual BiTE® as an example for the multitargeting antigen-binding molecule according to the present invention. It has to be noted that the above considerations are based on flexible linkers with a high Ser and/or Gly content. The skilled person would have contemplated that less flexible likers may require even higher numbers of amino acids to ensure sufficient length to keep distance between the two adjacent target binding domains according to the present invention, in order to keep said target binding domains biologically functional.

It is envisaged that the bispecific antigen-binding molecules according to the present invention have cross-reactivity to, for example, cynomolgus monkey tumor antigens such as CS1, BCMA, CD20, CD22, FLT3, CD123, CLL1, MSLN and EpCAM. It is in particular envisaged in the context of the present invention that two targets can be addressed simultaneously by one multitargeting antigen-binding molecule simultaneously which dual targeting mitigates, for example, (i) the presence of soluble target which otherwise would “mask” the target on the target cell by binding the antibody-based drug without allowing any therapeutic effect (e.g. soluble BCMA) and (ii) low cell surface BCMA expression risk in the course of antigen loss as the driving factor for tumor escape, as generally described herein.

For example, a multitargeting antigen-binding molecule according to the present invention such as a construct directed against CS1 as TAA1 and BCMA as TAA2 is suitable for use in the treatment of multiple myeloma (MM). The presently presented multitargeting antigen-binding molecule is particularly suitable to achieve efficacious exposure despite the presence of soluble target (e.g. BCMA), and antigen loss may lead to resistance. BCMA and CS1 are good combination partners due to their broad MM expression and limited normal expression relative to other MM antigens (e.g., CD38). A BCMAxCS1 HLE (half-life extended) dual BiTE® antigen-binding molecule as disclosed herein as an illustrative example, is manufacturable, has an acceptable safety profile, shows increased efficacy as the multitargeting BiTE® antigen-binding molecule induces lysis of CS1- and/or BCMA-expressing cells and is active in the presence of high concentrations of sBCMA and/or sCS1.

It is especially envisaged in the context of the present invention that a multitargeting antigen-binding molecule which preferably addresses two different target cell surface antigens thereby is very specific for its target cell and, therefore, preferably safe in its therapeutic use. This has been demonstrated in a cynomolgus toxicology study. Exemplary CS1xBCMA HLE dual BiTE® antigen-binding molecule is typically well tolerated and also typically no cytokine release syndrome occurs (CRS, a typical but severe side effect for T cell redirecting therapy) despite CS1 target expression in periphery. Peripheral CS1-expressing NK and T cells are preferably unaffected. Histopathological findings for the multitargeting BiTE® antigen-binding molecule are comparable to findings on a BiTE® antigen-binding molecule with only a binding domain to TAA BCMA. Further, multitargeting BiTE® antigen-binding molecule-mediated depletion of plasma cells from periphery and BM is typically observed and correlated with exposure. Even further, when J chain mRNA levels are plotted against BiTE® concentration of CS1 HLE BiTE® antigen-binding molecule versus BCMA HLE BiTE® antigen-binding molecule vs. monospecific CS1xBCMA HLE BiTE® antigen-binding molecule, the data preferably demonstrates that the exemplary CS1xBCMA HLE BiTE® antigen-binding molecule induces deeper target cell depletion than either CS1 HLE BiTE® antigen-binding molecule or BCMA HLE BiTE® which indicates better clinical efficacy of multitargeting antigen-binding molecules according to the present invention. Mesothelin, also known as MSLN, is a 40 kDa protein that is expressed in mesothelial cells. Mesothelin is a tumor differentiation antigen that is normally present on the mesothelial cells lining the pleura, peritoneum and pericardium. As MSLN is overexpressed in several cancers and is immunogenic, the protein could be exploited as tumor marker or as the antigenic target of a therapeutic cancer vaccine

Preferred target cell surface antigens in the context of the present invention are CS1, BCMA, CD20, CD22, FLT3, CD123, CLL1, MSLN, CDH3 and EpCAM. Typically, target cell surface antigens in the context of the present invention are tumor associated antigens (TAA). CS1 is a member of the CD2 subset of immunoglobulin superfamily (IgSF) expressed on NK, T and stimulated B cells. B-cell maturation antigen (BCMA or BCM), also known as tumor necrosis factor receptor superfamily member 17 (TNFRSF17), is a protein that is expressed in mature B lymphocytes. B-lymphocyte antigen CD20 or CD20 is expressed on the surface of all B-cells beginning at the pro-B phase (CD45R+, CD117+) and progressively increasing in concentration until maturity. CD22, or cluster of differentiation-22, is a molecule belonging to the SIGLEC family of lectins. It is found on the surface of mature B cells and to a lesser extent on some immature B cells. Fins like tyrosine kinase 3 (FLT3) is also known as Cluster of differentiation antigen 135 (CD135), receptor-type tyrosine-protein kinase FLT3, or fetal liver kinase-2 (Flk2). FLT3 is a cytokine receptor which belongs to the receptor tyrosine kinase class III. CD135 is the receptor for the cytokine Flt3 ligand (FLT3L). The FLT3 gene is frequently mutated in acute myeloid leukemia (AML). The interleukin-3 receptor (CD123) is a molecule found on cells which helps transmit the signal of interleukin-3, a soluble cytokine important in the immune system. C-type lectin-like receptor (CLL1), also known as CLEC12A, or as MICL. It contains an ITIM motif in cytoplasmic tail that can associate with signaling phosphatases SHP-1 and SHP-2. Human MICL is expressed as a monomer primarily on myeloid cells, including granulocytes, monocytes, macrophages and dendritic cells and is associated with AML. Mesothelin (MSLN) is a 40 kDa protein that is expressed in mesothelial cells and overexpressed in several human tumors. Cadherin-3 (CDH3), also known as P-Cadherin, is a calcium-dependent cell-cell adhesion glycoprotein composed of five extracellular cadherin repeats, a transmembrane region and a highly conserved cytoplasmic tail. It is associated with some types of tumors. Epithelial cell adhesion molecule (EpCAM) is a transmembrane glycoprotein mediating Ca2+-independent homotypic cell-cell adhesion in epithelia. EpCAM has oncogenic potential and appears to play a role in tumorigenesis and metastasis of carcinomas.

Further, it is envisaged as optionally but advantageously in the context of the present invention that the multitargeting antigen-binding molecule is provides with a fourth domain, typically a scFc domain, i.e. a HLE, antigen-binding molecule enables intravenous dosing that is administrated only once every week, once every two weeks, once every three weeks or even once every four weeks, or less frequently.

In order to determine the epitope(s) of preferred multitargeting antigen-binding molecules according to the present invention directed, e.g. to the CD20 epitope, mapping was conducted as described herein. The human CD20 protein extracellular region was divided into two parts: (1) extracellular loop 1 (ECL1, amino acids 72 to 84, see references in Example 17), designated E1, and extracellular loop 2 (ECL2), designated E2. The extracellular loop 1 (E1) was further divided into two subparts, designated ElA (aa 72 to 79) and ElB (aa 80 to 84). The extracellular loop 2 (E2, aa 142 to 188) was further divided into four subparts, designated E2A (aa 142 to 161), E2B (aa 162 to 166), E2C (aa 167 to 175) and E2D (aa 176 to 188). It was surprisingly found that CD20 antigen-binding molecules, both mono and dual targeting, show preferably higher cytotoxic activity when binding (i) to the ElA and the E2B and E2C epitope or (ii) to the E2 Å and E2B epitope. Correspondingly, for the purpose of epitope characterization the human CD22 protein extracellular region was divided into seven parts: V (aa 20-142 as specified in Uniprot P20273+RPFP), C2-1 (aa 143-241 as specified in Uniprot P20273+LNVKHT), C2-2 (aa 242-330 as specified in Uniprot P20273+VQYA), C2-3 (aa 331-418 as specified in Uniprot P20273+YP), C2-4 (aa 419-504 as specified in Uniprot P20273+VQYA), C2-5 (aa 505-592 as specified in Uniprot P20273+KAWTLEVLYA) and C2-6 (aa 593-687 as specified in Uniprot P20273+VYYSPETIGRR). It was surprisingly found that CD22 antigen-binding molecules, both mono and dual targeting, show preferably higher cytotoxic activity when binding to the C2-1 epitope.

It is particular surprising that a multispecific antigen-binding molecule according to the present invention is capable, despite the short linker between the target binding domains, to bind, preferably simultaneously to two different targets. Simultaneous binding has been demonstrated herein for several targets. However, this is surprising given the typically typical distance between the targets. For example, CD20 comprises two small extra cellular domains of only 13 aa (E1) and 47 aa (E2). In contrast, CD22 comprises a 7 Ig domain long extracellular domain with 676 aa. However, despite the significantly different extracellular size and setup, a multispecific antigen-binding molecule according to the present intention may successfully address both TAAs CD20 and CD22 at the same time for the benefit of increased efficacy and less toxicity. This is preferably achieved if the

The antigen-binding molecule of the present invention is preferably multitargeting and bispecific. However, also monospecific (for one target) and bispecific antigen-binding molecules which comprisc (i) a first binding domain against one cell surface target antigen and a second binding domain which is an effector binding domain which binds preferably to CD3c, or (ii) a first and a second binding domain against the same cell surface target and a third binding domain which is an effector binding domain which binds preferably to CD3e is encompassed by the present invention, wherein the cell surface target antigen is selected from CS1, CD20, CD22, CD123 and CLL1. For example, a CS1xCD3 bispecific antigen-binding molecule, i.e. which is directed to CS1 as only target cell surface antigen next to the effector CD3, has shown comparable efficacy in terms of EC₅₀ values than BCMAxCD3 antibody which is in clinical assessment and evaluation since a longer time. Also, e.g. CD20 antigen-binding molecules as described herein feature high activity against CD20 bearing targets in terms of EC50 values, in particular if they address specific epitopes as disclosed herein.

It is envisaged in the context of the present invention, that preferred multispecific antigen-binding molecules do not only show a favorable ratio of cytotoxicity to affinity, but additionally show sufficient stability characteristics in order to facilitate practical handling in formulating, storing and administrating said constructs. Sufficient stability is, for example, characterized by a high monomer content (i.e. non-aggregated and/or non-associated, native molecule) after standard preparation, such as at least 65% as determined by preparative size exclusion chromatography (SEC), more preferably at least 70% and even more preferably at least 75%. Also, the turbidity measured, e.g., at 340 nm as optical absorption at a concentration of 2.5 mg/ml should, preferably, be equal to or lower than 0.025, more preferably 0.020, e.g., in order to conclude to the essential absence of undesired aggregates. Advantageously, high monomer content is maintained after incubation in stress conditions such as freeze/thaw or incubation at 37 or 40° C. Even more, multispecific antigen-binding molecules according to the present invention typically have a thermal stability which is at least comparable or even higher than that of bispecific antigen-binding molecules which have only one target binding domain but otherwise comprise a CD3 binding domain and, optionally, a half-life extending scFc domain, i.e. which are structurally less complex. The skilled person would expect that a more structurally complex protein-based molecule was less prone to thermal and other degradation, i.e. be less thermal stable. However, surprisingly the contrary is the case, e.g., a CS1xBCMA or BCMAxCS1 multispecific antigen-binding molecule according to the present invention shows higher thermal stability, less monomer decrease after storage, higher monomer percentage after three freeze thaw cycles and higher protein homogeneity than a CS1 or a BCMA bispecific monotargeting antigen-binding molecule as disclosed herein. The same applies, for example, to the CD123xFLT3 multispecific antigen-binding molecules as disclosed herein, with respect to FTL3 bispecific antigen-binding molecules as disclosed herein.

Thus, the present invention provides a multispecific antigen-binding molecule comprising:

(i) the first binding domain specifically binds to a first target cell surface antigen (e.g. TAA1),
(ii) the second binding domain specifically binds to a second target cell surface antigen (e.g. TAA2), and
(iii) the third binding domain binds to an extracellular epitope of the human and/or the Macaca CD3ε chain, wherein the first, second and third binding domain are arranged in an amino to carboxyl order, and wherein the first binding domain and the second binding domain are linked by a peptide linker having a length of 5 to 25, preferably 5 to 18 or 6 to 16 amino acids, and optionally
(iv) a fourth domain which comprises two polypeptide monomers, each comprising a hinge, a CH2 and a CH3 domain, wherein said two polypeptide monomers are fused to each other via a peptide linker.

In an embodiment, the present invention provides a multispecific antigen-binding molecule comprising all four such domains. In a preferred embodiment, the domains under (i), (ii), (iii) and (iv) are arranged in an N to C orientation (format 2). However, alternatively, the multispecific antigen-binding molecule may have the domains arranged in the order (ii), (iii), (iv) (format 1) and (i), or (ii), (iv), (i) and (iii) (format 3) in an N to C orientation. Surprisingly, all arrangements (a) provide significant efficacy in terms of on target cytotoxicity and (b) are producible in acceptable product quality. As a general requirement for the multitargeting bispecific antigen-binding molecule of the present invention, one target binding domain has to be located adjacently N-terminally to the effector CD3 binding domain in order to act as a bispecific entity and, thereby, form a cytolytic synapse between the—preferably double positive-target cell and the effector T-cell.

The term “polypeptide” is understood herein as an organic polymer which comprises at least one continuous, unbranched amino acid chain. In the context of the present invention, a polypeptide comprising more than one amino acid chain is likewise envisaged. An amino acid chain of a polypeptide typically comprises at least 50 amino acids, preferably at least 100, 200, 300, 400 or 500 amino acids. It is also envisaged in the context of the present invention that an amino acid chain of a polymer is linked to an entity which is not composed of amino acids.

The term “antigen-binding polypeptide” according to the present invention is preferably a polypeptide which immunospecifically binds to its target or antigen. It typically comprises the heavy chain variable region (VH) and/or the light chain variable region (VL) of an antibody, or comprises domains derived therefrom. A polypeptide according to the invention comprises the minimum structural requirements of an antibody which allow for immunospecific target binding. This minimum requirement may e.g. be defined by the presence of at least three light chain CDRs (i.e. CDR1, CDR2 and CDR3 ofthe VL region) and/or three heavy chain CDRs (i.e. CDR1, CDR2 and CDR3 of the VH region), preferably of all six CDRs. A T-cell engaging polypeptide may hence be characterized by the presence of three or six CDRs in either one or both binding domains, and the skilled person knows where (in which order) those CDRs are located within the binding domain. Typically, an “antigen-binding molecule” is understood as an “antigen-binding polypeptide” in the context of the present invention.

Alternatively, in the context of the present invention, an antigen-binding polypeptide corresponds to an “antibody construct” which typically refers to a molecule in which the structure and/or function is/are based on the structure and/or function of an antibody, e.g., of a full-length or whole immunoglobulin molecule. An antigen-binding molecule is hence capable of binding to its specific target or antigen and/or is/are drawn from the variable heavy chain (VH) and/or variable light chain (VL) domains of an antibody or fragment thereof. Furthermore, the domain which binds to its binding partner according to the present invention is understood herein as a binding domain of an antigen-binding molecule according to the invention. Typically, a binding domain according to the present invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs. An alternative approach to define the minimal structure requirements of an antibody is the definition of the epitope of the antibody within the structure of the specific target, respectively, the protein domain of the target protein composing the epitope region (epitope cluster) or by reference to a specific antibody competing with the epitope of the defined antibody. The antibodies on which the constructs according to the invention are based include for example monoclonal, recombinant, chimeric, deimmunized, humanized and human antibodies.

The binding domain of an antigen-binding molecule according to the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding domain. Additional examples for the format of antibody fragments, antibody variants or binding domains include (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library). Examples for embodiments of antigen-binding molecules according to the invention are e.g. described in WO 00/006605, WO 2005/040220, WO 2008/119567, WO 2010/037838, WO 2013/026837, WO 2013/026833, US 2014/0308285, US 2014/0302037, WO 2014/144722, WO 2014/151910, and WO 2015/048272.

Also, within the definition of “binding domain” or “domain which binds” are fragments of full-length antibodies, such as VH, VHH, VL, (s)dAb, Fv, Fd, Fab, Fab′, F(ab′)2 or “r IgG” (“half antibody”). Antigen-binding molecules according to the invention may also comprise modified fragments of antibodies, also called antibody variants, such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “multibodies” such as triabodies or tetrabodies, and single domain antibodies such as nanobodies or single variable domain antibodies comprising merely one variable domain, which may be VHH, VH or VL, that specifically bind an antigen or epitope independently of other V regions or domains.

As used herein, the terms “single-chain Fv,” “single-chain antibodies” or “scFv” refer to single polypeptide chain antibody fragments that comprise the variable regions from both the heavy and light chains, but lack the constant regions. Generally, a single-chain antibody further comprises a polypeptide linker between the VH and VL domains which enables it to form the desired structure which would allow for antigen binding. Single chain antibodies are discussed in detail by Pluckthun in The Pharmacology of Monoclonal Antibodies, vol. 113, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, and/or humanized and/or synthetic.

Furthermore, the definition of the term “antigen-binding molecule” includes preferably polyvalent/multivalent constructs and, thus, bispecific molecules, wherein bispecific means that they specifically bind to two cell typs comprising distinctive antigenic structures, i.e. target cells and effector cells. As the antigen-binding molecules of the present invention are preferably multitargeting, they are typically as well as polyvalent/multivalent molecules, which specifically bind more than two antigenic structures, preferably. three, through distinct binding domains in the context of the present invention which are two target binding domains and one CD3 binding domain. Moreover, the definition of the term “antigen-binding molecule” includes molecules consisting of only one polypeptide chain as well as molecules consisting of more than one polypeptide chain, which chains can be either identical (homodimers, homotrimers or homo oligomers) or different (heterodimer, heterotrimer or heterooligomer). Such molecules comprising more than one polypeptide chain, i.e. typically two chains, have these chains typically attached to each other as heterodimers via charged pair binding, e.g. within a heteroFc entity which serves as a half-life extending moiety e.g. in C-terminal position of the CD3 binder as described herein. Examples for the above identified antigen-binding molecules, e.g. antibody-based molecules are described inter alia in Harlow and Lane, Antibodies a laboratory manual, CSHL Press (1988) and Using Antibodies: a laboratory manual, CSHL Press (1999), Kontermann and Dübel, Antibody Engineering, Springer, 2nd ed. 2010 and Little, Recombinant Antibodies for Immunotherapy, Cambridge University Press 2009.

The term “bispecific” as used herein refers to an antigen-binding molecule which is “at least bispecific”, i.e., it addresses two different cell types, i.e. target an effector cells, and comprises at least a first binding domain and a second binding domain, wherein at least one binding domain binds to an antigen or target selected preferably from CS1, BCMA, CD20, CD22, FLT3, CD123, MSLN, CLL1 and EpCAM, and another binding domain of the same molecule binds to another antigen or target (here: CD3). Accordingly, antigen-binding molecules according to the invention comprise specificities for at least two different antigens or targets. For example, one domain does preferably not bind to an extracellular epitope of CD3e of one or more of the species as described herein.

The term “target cell surface antigen” refers to an antigenic structure expressed by a cell and which is present at the cell surface such that it is accessible for an antigen-binding molecule as described herein. A preferred target cell surface antigen in the context of the present invention is a tumor associated antigen (TAA). It may be a protein, preferably the extracellular portion of a protein, or a carbohydrate structure, preferably a carbohydrate structure of a protein, such as a glycoprotein. It is preferably a tumor antigen. The term “bispecific antigen-binding molecule” of the invention also encompasses multispecific antigen-binding molecules such as trispecific antigen-binding molecules, the latter ones including three binding domains, or constructs having more than three (e.g. four, five . . . ) specificities.

Preferred in the context of the present invention is a molecule which is “multispecific”, which is understood herein to be “at least bispecific”. In this regard, a multispecific molecule such as an antigen-binding molecule is specific for an effector such as CD3, more preferably CD3e, and at least two target cell surface antigens. Said specificity is conferred by respective binding domains as defined herein. Typically, “multispecific” refers to a molecule which is specific for two different target cell surface effectors as such multi-specificity confers to preferred properties of a multispecific antigen-binding molecule according to the present invention, namely mitigation of antigen loss and increase of the therapeutic window or higher tolerability.

Given that the antigen-binding molecules according to the invention are (at least) bispecific, they do not occur naturally and they are markedly different from naturally occurring products. A “bispecific” antigen-binding molecule or immunoglobulin is hence an artificial hybrid antibody or immunoglobulin having at least two distinct binding sides with different specificities. Bispecific antigen-binding molecules can be produced by a variety of methods including fusion of hybridomas or linking of Fab′ fragments. See, e.g., Songsivilai & Lachmann, Clin. Exp. Immunol. 79:315-321 (1990).

The at least three binding domains and the variable domains (VH/VL) of the antigen-binding molecule of the present invention typically comprise peptide linkers (spacer peptides). The term “peptide linker” comprises in accordance with the present invention an amino acid sequence by which the amino acid sequences of one (variable and/or binding) domain and another (variable and/or binding) domain of the antigen-binding molecule of the invention are linked with each other. The peptide linker between the first and the second binding domain, which are capable to bind simultaneously to two targets, which are preferably different targets (e.g. TAA1 and TAA2), are preferably flexible and of limited length, e.g. of 5, 6, 7, 8, 9, 10, 11, 12, 13, 14, 15, 16, 17 or 18 amino acids. The peptide linkers can also be used to fuse the third domain to the other domains of the antigen-binding molecule of the invention. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the antigen-binding molecule of the invention. However, typically the linker between the first and the second target binding domain differs from the intra-binder linker which links the VH and VL within the target binding domain. Said difference is the linker between the first and the second binding domain having one amino acid more than intra-binder linkers, e.g. six and five amino acids, respectively, such as SGGGGS versus GGGGS. This confers surprisingly flexibility and stability at the same time in the specific antigen-binding molecule format as described herein.

The antigen-binding molecules of the present invention are preferably “in vitro generated antigen-binding molecules”. This term refers to an antigen-binding molecule according to the above definition where all or part of the variable region (e.g., at least one CDR) is generated in a non-immune cell selection, e.g., an in vitro phage display, protein chip or any other method in which candidate sequences can be tested for their ability to bind to an antigen. This term thus preferably excludes sequences generated solely by genomic rearrangement in an immune cell in an animal. A “recombinant antibody” is an antibody made through the use of recombinant DNA technology or genetic engineering.

The term “monoclonal antibody” (mAb) or monoclonal antigen-binding molecule as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic side or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance analysis, e.g. Biacore™ to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the Biacore system can be used to increase the efficiency of phage antibodies which bind to an epitope of a target cell surface antigen (Schier, Human Antibodies Hybridomas 7 (1996), 97-105; Malmborg, J. Immunol. Methods 183 (1995), 7-13).

Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antigen-binding molecules include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dubel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antigen-binding molecules, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the antigen-binding molecules involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e. g. 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e. g. binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human CS1, BCMA, CD20, CD22, FLT3, CD123, MSLN, CLL1 or EpCAM. Such contact residues and neighbouring residues are candidates for substitution according to the techniques elaborated herein. Once such variants are generated, the panel of variants is subjected to screening as described herein and antibodies with superior properties in one or more relevant assays may be selected for further development.

The monoclonal antibodies and antigen-binding molecules of the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Momson et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. ScL U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antigen-binding molecule, antibody fragment or antibody variant may also be modified by specific deletion of human T cell epitopes (a method called “deimmunization”) by the methods disclosed for example in WO 98/52976 or WO 00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. MoI. Biol. 227:776-798; Cook, G. P. et al. (1995) Immunol. Today Vol. 16 (5): 237-242; and Tomlinson et al. (1995) EMBO J. 14: 14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, antigen-binding molecules, variants or fragments thereof (such as Fv, Fab, Fab′, F(ab′)2 or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

The term “human antibody”, “human antigen-binding molecule” and “human binding domain” includes antibodies, antigen-binding molecules and binding domains having antibody regions such as variable and constant regions or domains which correspond substantially to human germline immunoglobulin sequences known in the art, including, for example, those described by Kabat et al. (1991) (loc. cit.). The human antibodies, antigen-binding molecules or binding domains of the invention may include amino acid residues not encoded by human germline immunoglobulin sequences (e.g., mutations introduced by random or site-specific mutagenesis in vitro or by somatic mutation in vivo), for example in the CDRs, and in particular, in CDR3. The human antibodies, antigen-binding molecules or binding domains can have at least one, two, three, four, five, or more positions replaced with an amino acid residue that is not encoded by the human germline immunoglobulin sequence. The definition of human antibodies, antigen-binding molecules and binding domains as used herein also contemplates fully human antibodies, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences.

In some embodiments, the antigen-binding molecules of the invention are “isolated” or “substantially pure” antigen-binding molecules. “Isolated” or “substantially pure”, when used to describe the antigen-binding molecules disclosed herein, means an antigen-binding molecule that has been identified, separated and/or recovered from a component of its production environment. Preferably, the antigen-binding molecule is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The antigen-binding molecules may e.g. constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an antigen-binding molecule in a wide variety of organisms and/or host cells that are known in the art. In preferred embodiments, the antigen-binding molecule will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antigen-binding molecule will be prepared by at least one purification step.

The term “binding domain” characterizes in connection with the present invention a domain which (specifically) binds to/interacts with/recognizes a given target epitope or a given target side on the target molecules (antigens), e.g. CS1, BCMA, CD20, CD22, FLT3, CD123, CLL1, MSLN, or EpCAM, and CD3, respectively. The structure and function of the first and/or second binding domain (recognizing CS1, BCMA, CD20, CD22, FLT3, CD123, CLL1, MSLN, or EpCAM), and preferably also the structure and/or function of the effector binding domain (typically the third binding domain recognizing CD3), is/are based on the structure and/or function of an antibody, e.g. of a full-length or whole immunoglobulin molecule, and/or is/are drawn from the variable heavy chain (VH) and/or variable light chain (VL) domains of an antibody or fragment thereof. Preferably the target cell surface antigen(s) binding domain(s) is/are characterized by the presence of three light chain CDRs (i.e. CDR1, CDR2 and CDR3 of the VL region) and/or three heavy chain CDRs (i.e. CDR1, CDR2 and CDR3 of the VH region). The effector (typically CD3) binding domain preferably also comprises the minimum structural requirements of an antibody which allow for the target binding. More preferably, the second binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that the first and/or second binding domain is produced by or obtainable by phage-display or library screening methods rather than by grafting CDR sequences from a pre-existing (monoclonal) antibody into a scaffold.

According to the present invention, binding domains are in the form of one or more polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

The term “polypeptide” as used herein describes a group of molecules, which usually consist of more than 30 amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is effected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

Preferably the binding domain which binds to any of CS1, BCMA, CD20, CD22, FLT3, CD123, CLL1, MSLN, and EpCAM, and/or the binding domain which binds to CD3s is/are human binding domains. Antibodies and antigen-binding molecules comprising at least one human binding domain avoid some of the problems associated with antibodies or antigen-binding molecules that possess non-human such as rodent (e.g. murine, rat, hamster or rabbit) variable and/or constant regions. The presence of such rodent derived proteins can lead to the rapid clearance of the antibodies or antigen-binding molecules or can lead to the generation of an immune response against the antibody or antigen-binding molecule by a patient. In order to avoid the use of rodent derived antibodies or antigen-binding molecules, human or fully human antibodies/antigen-binding molecules can be generated through the introduction of human antibody function into a rodent so that the rodent produces fully human antibodies.

The ability to clone and reconstruct megabase-sized human loci in yeast artificial chromosomes YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antigen-binding molecules are expected to minimize the immunogenic and allergic responses intrinsic to mouse or mouse-derivatized mAbs and thus to increase the efficacy and safety of the administered antibodies/antigen-binding molecules. The use of fully human antibodies or antigen-binding molecules can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with YACs containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions may recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouseanimals is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3,068,180 B2, 3068506 B2, and 3068507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773288 and 843961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

Human anti-mouse antibody (HAMA) responses have led the industry to prepare chimeric or otherwise humanized antibodies. It is however expected that certain human anti-chimeric antibody (HACA) responses will be observed, particularly in chronic or multi-dose utilizations of the antibody. Thus, it would be desirable to provide antigen-binding molecules comprising a human binding domain against CS1, BCMA, CD20, CD22, FLT3, CD123, CLL1, MSLN, CDH3 or EpCAM and a human binding domain against CD3s in order to vitiate concerns and/or effects of HAMA or HACA response.

The terms “(specifically) or (immune-specifically) binds to”, (specifically) recognizes”, “is (specifically) directed to”, and “(specifically) reacts with” mean in accordance with this invention that a binding domain, preferably by means of its paratope, interacts or specifically interacts with a given epitope or a given target side on the target molecules (antigens), here preferably CS1, BCMA, CD20, CD22, FLT3, CD123, CLL1, MSLN, CDH3 or EpCAM, and CD3s, respectively.

In the context of the present invention, a paratope is understood as an antigen-binding site which is a part of a polypeptide as described herein and which recognizes and binds to an antigen. A paratope is typically a small region of about at least 5 amino acids. A paratope as understood herein typically comprises parts of antibody-derived heavy (VH) and light chain (VL) sequences. Each binding domain of a polypeptide according to the present invention is provided with a paratope comprising a set of 6 complementarity-determining regions (CDR loops) with three of each being comprised within the antibody-derived VH and VL sequence, respectively.

In the context of the present invention, an antigen-binding molecule, i.e. preferably a polypeptide, of the present invention binds to its respective target structure in a particular manner. Preferably, a polypeptide according to the present invention comprises one paratope per binding domain which specifically or immunospecifically binds to”, “(specifically or immunospecifically) recognizes”, or “(specifically or immunospecifically) reacts with” its respective target structure. This means in accordance with this invention that a polypeptide or a binding domain thereof interacts or (immuno-)specifically interacts with a given epitope on the target molecule (antigen) and CD3, respectively. This interaction or association occurs more frequently, more rapidly, with greater duration, with greater affinity, or with some combination of these parameters, to an epitope on the specific target than to alternative substances (non-target molecules). Because of the sequence similarity between homologous proteins in different species, an antibody construct or a binding domain that immunspecifically binds to its target (such as a human target) may, however, cross-react with homologous target molecules from different species (such as, from non-human primates). The term “specific/immunospecific binding” can hence include the binding of an antibody construct or binding domain to epitopes and/or structurally related epitopes in more than one species. The term “(immuno-) selectively binds does exclude the binding to structurally related epitopes.

The term “epitope” refers to a side on an antigen to which a binding domain, such as an antibody or immunoglobulin, or a derivative, fragment or variant of an antibody or an immunoglobulin, specifically binds. An “epitope” is antigenic and thus the term epitope is sometimes also referred to herein as “antigenic structure” or “antigenic determinant”. Thus, the binding domain is an “antigen interaction side”. Said binding/interaction is also understood to define a “specific recognition”.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically, a conformational epitope comprises an increased number of amino acids relative to a linear epitope. With regard to recognition of conformational epitopes, the binding domain recognizes a three-dimensional structure of the antigen, preferably a peptide or protein or fragment thereof (in the context of the present invention, the antigenic structure for one of the binding domains is comprised within the target cell surface antigen protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

A method for epitope mapping is described in the following: When a region (a contiguous amino acid stretch) in the human CS1, BCMA, CD20, CD22, FLT3, CD123, CLL1, MSLN, or EpCAM protein is exchanged or replaced with its corresponding region of a non-human and non-primate CS1, BCMA, CD20, CD22, FLT3, CD123, CLL1, MSLN, or EpCAM (e.g., mouse CS1, BCMA, CD20, CD22, FLT3, CD123, CLL1, MSLN, or EpCAM, but others like chicken, rat, hamster, rabbit etc. may also be conceivable), a decrease in the binding of the binding domain is expected to occur, unless the binding domain is cross-reactive for the non-human, non-primate CS1, BCMA, CD20, CD22, FLT3, CD123, CLL1, MSLN, CDH3 or EpCAM used. Said decrease is preferably at least 10%, 20%, 30%, 40%, or 50%; more preferably at least 60%, 70%, or 80%, and most preferably 90%, 95% or even 100% in comparison to the binding to the respective region in the human CS1, BCMA, CD20, CD22, FLT3, CD123, CLL1, MSLN, or EpCAM protein, whereby binding to the respective region in the human CS1, BCMA, CD20, CD22, FLT3, CD123, CLL1, MSLN, or EpCAM protein is set to be 100%. It is envisaged that the aforementioned human CS1, BCMA, CD20, CD22, FLT3, CD123, CLL1, MSLN, or EpCAM/non-human CS1, BCMA, CD20, CD22, FLT3, CD123, CLL1, MSLN, or EpCAM chimeras are expressed in CHO cells. It is also envisaged that the human CS1, BCMA, CD20, CD22, FLT3, CD123, CLL1, MSLN, or EpCAM/non-human CS1, BCMA, CD20, CD22, FLT3, CD123, CLL1, MSLN, or EpCAM chimeras are fused with a transmembrane domain and/or cytoplasmic domain of a different membrane-bound protein such as EpCAM.

In an alternative or additional method for epitope mapping, several truncated versions of the human CS1, BCMA, CD20, CD22, FLT3, CD123, CLL1, MSLN, or EpCAM extracellular domain can be generated in order to determine a specific region that is recognized by a binding domain. In these truncated versions, the different extracellular CS1, BCMA, CD20, CD22, FLT3, CD123, CLL1, MSLN, or EpCAM domains/sub-domains or regions are stepwise deleted, starting from the N-terminus. It is envisaged that the truncated CS1, BCMA, CD20, CD22, FLT3, CD123, CLL1, MSLN, or EpCAM versions may be expressed in CHO cells. It is also envisaged that the truncated CS1, BCMA, CD20, CD22, FLT3, CD123, CLL1, MSLN, or EpCAM versions may be fused with a transmembrane domain and/or cytoplasmic domain of a different membrane-bound protein such as EpCAM. It is also envisaged that the truncated CS1, BCMA, CD20, CD22, FLT3, CD123, CLL1, MSLN, or EpCAM versions may encompass a signal peptide domain at their N-terminus, for example a signal peptide derived from mouse IgG heavy chain signal peptide. It is furthermore envisaged that the truncated CS1, BCMA, CD20, CD22, FLT3, CD123, CLL1, MSLN, or EpCAM versions may encompass a v5 domain at their N-terminus (following the signal peptide) which allows verifying their correct expression on the cell surface. A decrease or a loss of binding is expected to occur with those truncated CS1, BCMA, CD20, CD22, FLT3, CD123, CLL1, MSLN, or EpCAM versions which do not encompass any more the CS1, BCMA, CD20, CD22, FLT3, CD123, CLL1, MSLN, or EpCAM region that is recognized by the binding domain. The decrease of binding is preferably at least 10%, 20%, 30%, 40%, 50%; more preferably at least 60%, 70%, 80%, and most preferably 90%, 95% or even 100%, whereby binding to the entire human CS1, BCMA, CD20, CD22, FLT3, CD123, CLL1, MSLN, or EpCAM protein (or its extracellular region or domain) is set to be 100.

A further method to determine the contribution of a specific residue of CS1, BCMA, CD20, CD22, FLT3, CD123, CLL1, MSLN, or EpCAM to the recognition by an antigen-binding molecule or binding domain is alanine scanning (see e.g. Morrison K L & Weiss G A. Cur Opin Chem Biol. 2001 June; 5(3):302-7), where each residue to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired. Alanine scanning is a mature technology which has been used for a long period of time.

The interaction between the binding domain and the epitope or the region comprising the epitope implies that a binding domain exhibits appreciable affinity for the epitope/the region comprising the epitope on a particular protein or antigen (here: CS1, BCMA, CD20, CD22, FLT3, CD123, CLL1, MSLN, CDH3 or EpCAM and CD3, respectively) and, generally, does not exhibit significant reactivity with proteins or antigens other than the CS1, BCMA, CD20, CD22, FLT3, CD123, CLL1, MSLN, CDH3 or EpCAM or CD3. “Appreciable affinity” includes binding with an affinity of about 10⁻⁶ M (KD) or stronger. Preferably, binding is considered specific when the binding affinity is about 10⁻¹² to 10⁻⁸ M, 10-12 to 10⁻⁹ M, 10-12 to 10-10 M, 10-11 to 10⁻⁸ M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than the CS1, BCMA, CD20, CD22, FLT3, CD123, CLL1, MSLN, CDH3 or EpCAM or CD3. Preferably, a binding domain of the invention does not essentially or substantially bind to proteins or antigens other than CS1, BCMA, CD20, CD22, FLT3, CD123, CLL1, MSLN, or EpCAM or CD3 (i.e., the first binding domain is not capable of binding to proteins other than CS1, BCMA, CD20, CD22, FLT3, CD123, CLL1, MSLN, CDH3 or EpCAM and the second binding domain is not capable of binding to proteins other than CD3). It is an envisaged characteristic of the antigen-binding molecules according to the present invention to have superior affinity characteristics in comparison to other HLE formats. Such a superior affinity, in consequence, suggests a prolonged half-life in vivo. The longer half-life of the antigen-binding molecules according to the present invention may reduce the duration and frequency of administration which typically contributes to improved patient compliance. This is of particular importance as the antigen-binding molecules of the present invention are particularly beneficial for highly weakened or even multimorbid cancer patients.

The term “does not essentially/substantially bind” or “is not capable of binding” means that a binding domain of the present invention does not bind a protein or antigen other than the CS1, BCMA, CD20, CD22, FLT3, CD123, CLL1, MSLN, or EpCAM or CD3, i.e., does not show reactivity of more than 30%, preferably not more than 20%, more preferably not more than 10%, particularly preferably not more than 9%, 8%, 7%, 6% or 5% with proteins or antigens other than CS1, BCMA, CD20, CD22, FLT3, CD123, CLL1, MSLN, CDH3 or EpCAM or CD3, whereby binding to the CS1, BCMA, CD20, CD22, FLT3, CD123, CLL1, MSLN, CDH3 or EpCAM or CD3, respectively, is set to be 100%.

Specific binding is believed to be effected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-side with its specific antigen may result in a simple binding of said side to the antigen. Moreover, the specific interaction of the antigen-interaction-side with its specific antigen may alternatively or additionally result in the initiation of a signal, e.g. due to the induction of a change of the conformation of the antigen, an oligomerization of the antigen, etc.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding site.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface. The variable domains of naturally occurring heavy and light chains each comprise four FRM regions (FR1, FR2, FR3, and FR4), largely adopting a R-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding side (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. MoI. Biol, 1987, 196: 901-917; and MacCallum et al., J. MoI. Biol, 1996, 262: 732). Still another standard for characterizing the antigen binding side is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. MoI. Biol., 1987, 196: 901; Chothia et al., Nature, 1989, 342: 877; Martin and Thornton, J. MoI. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some antigen-binding molecules, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding side. H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The CH domain most proximal to VH is usually designated as CH1. The constant (“C”) domains are not directly involved in antigen binding, but exhibit various effector functions, such as antibody-dependent, cell-mediated cytotoxicity and complement activation. The Fc region of an antibody is comprised within the heavy chain constant domains and is for example able to interact with cell surface located Fc receptors.

The sequence of antibody genes after assembly and somatic mutation is highly varied, and these varied genes are estimated to encode 10¹⁰ different antibody molecules (Immunoglobulin Genes, 2^{nd} ed., eds. Jonio et al., Academic Press, San Diego, Calif., 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

The term “Fc portion” or “Fc monomer” means in connection with this invention a polypeptide comprising at least one domain having the function of a CH2 domain and at least one domain having the function of a CH3 domain of an immunoglobulin molecule. As apparent from the term “Fc monomer”, the polypeptide comprising those CH domains is a “polypeptide monomer”. An Fc monomer can be a polypeptide comprising at least a fragment of the constant region of an immunoglobulin excluding the first constant region immunoglobulin domain of the heavy chain (CH1), but maintaining at least a functional part of one CH2 domain and a functional part of one CH3 domain, wherein the CH2 domain is amino terminal to the CH3 domain. In a preferred aspect of this definition, an Fc monomer can be a polypeptide constant region comprising a portion of the Ig-Fc hinge region, a CH2 region and a CH3 region, wherein the hinge region is amino terminal to the CH2 domain. It is envisaged that the hinge region of the present invention promotes dimerization. Such Fc polypeptide molecules can be obtained by papain digestion of an immunoglobulin region (of course resulting in a dimer of two Fc polypeptide), for example and not limitation. In another aspect of this definition, an Fc monomer can be a polypeptide region comprising a portion of a CH2 region and a CH3 region. Such Fc polypeptide molecules can be obtained by pepsin digestion of an immunoglobulin molecule, for example and not limitation. In one embodiment, the polypeptide sequence of an Fc monomer is substantially similar to an Fc polypeptide sequence of: an IgG₁ Fc region, an IgG₂ Fc region, an IgG₃ Fc region, an IgG₄ Fc region, an IgM Fc region, an IgA Fc region, an IgD Fc region and an IgE Fc region. (See, e.g., Padlan, Molecular Immunology, 31(3), 169-217 (1993)). Because there is some variation between immunoglobulins, and solely for clarity, Fc monomer refers to the last two heavy chain constant region immunoglobulin domains of IgA, IgD, and IgG, and the last three heavy chain constant region immunoglobulin domains of IgE and IgM. As mentioned, the Fc monomer can also include the flexible hinge N-terminal to these domains. For IgA and IgM, the Fc monomer may include the J chain. For IgG, the Fc portion comprises immunoglobulin domains CH2 and CH3 and the hinge between the first two domains and CH2. Although the boundaries of the Fc portion may vary an example for a human IgG heavy chain Fc portion comprising a functional hinge, CH2 and CH3 domain can be defined e.g. to comprise residues D231 (of the hinge domain—corresponding to D234 in Table 1 below) to P476, respectively L476 (for IgG₄) of the carboxyl-terminus of the CH3 domain, wherein the numbering is according to Kabat. The two Fc portion or Fc monomer, which are fused to each other via a peptide linker define the third domain of the antigen-binding molecule of the invention, which may also be defined as scFc domain.

In one embodiment of the invention it is envisaged that a scFc domain as disclosed herein, respectively the Fc monomers fused to each other are comprised only in the third domain of the antigen-binding molecule.

In line with the present invention an IgG hinge region can be identified by analogy using the Kabat numbering as set forth in Table 1. In line with the above, it is envisaged that for a hinge domain/region of the present invention the minimal requirement comprises the amino acid residues corresponding to the IgG1 sequence stretch of D231 D234 to P243 according to the Kabat numbering. It is likewise envisaged that a hinge domain/region of the present invention comprises or consists of the IgG1 hinge sequence DKTHTCPPCP (SEQ ID NO) (corresponding to the stretch D234 to P243 as shown in Table 1 below—variations of said sequence are also envisaged provided that the hinge region still promotes dimerization). In a preferred embodiment of the invention the glycosylation site at Kabat position 314 of the CH2 domains in the third domain of the antigen-binding molecule is removed by a N314X substitution, wherein X is any amino acid excluding Q. Said substitution is preferably a N314G substitution. In a more preferred embodiment, said CH2 domain additionally comprises the following substitutions (position according to Kabat) V321C and R309C (these substitutions introduce the intra domain cysteine disulfide bridge at Kabat positions 309 and 321).

It is also envisaged that the third domain of the antigen-binding molecule of the invention comprises or consists in an amino to carboxyl order: DKTHTCPPCP (SEQ ID NO) (i.e. hinge) —CH2-CH3-linker-DKTHTCPPCP (SEQ ID NO) (i.e. hinge) —CH2-CH3. The peptide linker of the aforementioned antigen-binding molecule is in a preferred embodiment characterized by the amino acid sequence Gly-Gly-Gly-Gly-Ser, i.e. Gly4Ser (SEQ ID NO: 1), or polymers thereof, i.e. (Gly4Ser)x, where x is an integer of 5 or greater (e.g. 5, 6, 7, 8 etc. or greater), 6 being preferred ((Gly4Ser)6). Said construct may further comprise the aforementioned substitutions: N314X, preferably N314G, and/or the further substitutions V321C and R309C. In a preferred embodiment of the antigen-binding molecules of the invention as defined herein before, it is envisaged that the second domain binds to an extracellular epitope of the human and/or the Macaca CD3ε chain.

TABLE 1
Kabat numbering of the amino acid residues of the hinge region

| IMGT numbering | IgG₁ amino acid | Kabat |
|---|---|---|
| for the hinge | translation | numbering |
| 1 | (E) | 226 |
| 2 | P | 227 |
| 3 | K | 228 |
| 4 | S | 232 |
| 5 | C | 233 |
| 6 | D | 234 |
| 7 | K | 235 |
| 8 | T | 236 |
| 9 | H | 237 |
| 10 | T | 238 |
| 11 | C | 239 |
| 12 | P | 240 |
| 13 | P | 241 |
| 14 | C | 242 |
| 15 | P | 243 |

In further embodiments of the present invention, the hinge domain/region comprises or consists of the IgG2 subtype hinge sequence ERKCCVECPPCP (SEQ ID NO), the IgG3 subtype hinge sequence ELKTPLDTTHTCPRCP (SEQ ID NO) or ELKTPLGDTTHTCPRCP (SEQ ID NO), and/or the IgG4 subtype hinge sequence ESKYGPPCPSCP (SEQ ID NO). The IgG1 subtype hinge sequence may be the following one EPKSCDKTHTCPPCP (as shown in Table 1 and SEQ ID NO). These core hinge regions are thus also envisaged in the context of the present invention.

The location and sequence of the IgG CH2 and IgG CD3 domain can be identified by analogy using the Kabat numbering as set forth in Table 2:

TABLE 2
Kabat numbering of the amino acid residues of the IgG CH2
and CH3 region

| IgG | CH2 aa | CH2 Kabat | CH3 aa | CH3 Kabat |
|---|---|---|---|---|
| subtype | translation | numbering | translation | numbering |
| IgG₁ | APE . . . KAK | 244 . . . 360 | GQP . . . PGK | 361 . . . 478 |
| IgG₂ | APP . . . KTK | 244 . . . 360 | GQP . . . PGK | 361 . . . 478 |
| IgG₃ | APE . . . KTK | 244 . . . 360 | GQP . . . PGK | 361 . . . 478 |
| IgG₄ | APE . . . KAK | 244 . . . 360 | GQP . . . PGK | 361 . . . 478 |

In one embodiment of the invention the emphasized bold amino acid residues in the CH3 domain of the first or both Fc monomers are deleted.

The peptide linker, by whom the polypeptide monomers (“Fc portion” or “Fc monomer”) of the third domain are fused to each other, preferably comprises at least 25 amino acid residues (25, 26, 27, 28, 29, 30 etc.). More preferably, this peptide linker comprises at least 30 amino acid residues (30, 31, 32, 33, 34, 35 etc.). It is also preferred that the linker comprises up to 40 amino acid residues, more preferably up to 35 amino acid residues, most preferably exactly 30 amino acid residues. A preferred embodiment of such peptide linker is characterized by the amino acid sequence Gly-Gly-Gly-Gly-Ser, i.e. Gly₄Ser (SEQ ID NO: 1), or polymers thereof, i.e. (Gly₄Ser)x, where x is an integer of 5 or greater (e.g. 6, 7 or 8). Preferably the integer is 6 or 7, more preferably the integer is 6.

In the event that a linker is used to fuse the first domain to the second domain, or the first or second domain to the third domain, this linker is preferably of a length and sequence sufficient to ensure that each of the first and second domains can, independently from one another, retain their differential binding specificities. For peptide linkers which connect the at least two binding domains (or two variable domains) in the antigen-binding molecule of the invention, those peptide linkers are preferred which comprise only a few number of amino acid residues, e.g. 12 amino acid residues or less. Thus, peptide linkers of 12, 11, 10, 9, 8, 7, 6 or 5 amino acid residues are preferred. An envisaged peptide linker with less than 5 amino acids comprises 4, 3, 2 or one amino acid(s), wherein Gly-rich linkers are preferred. A preferred embodiment of the peptide linker for a fusion the first and the second domain is depicted in SEQ ID NO:1. A preferred linker embodiment of the peptide linker for fusing the second and the third domain is a (Gly)₄-linker, also called G₄-linker.

A particularly preferred “single” amino acid in the context of one of the above described “peptide linker” is Gly. Accordingly, said peptide linker may consist of the single amino acid Gly. In a preferred embodiment of the invention a peptide linker is characterized by the amino acid sequence Gly-Gly-Gly-Gly-Ser, i.e. Gly₄Ser (SEQ ID NO: 1), or polymers thereof, i.e. (Gly₄Ser)x, where x is an integer of 1 or greater (e.g. 2 or 3). Preferred linkers are depicted in SEQ ID NOs: 1 to 12. The characteristics of said peptide linker, which comprise the absence of the promotion of secondary structures, are known in the art and are described e.g. in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers which furthermore do not promote any secondary structures are preferred. The linkage of said domains to each other can be provided, e.g., by genetic engineering, as described in the examples. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, N.Y., 2001).

In a preferred embodiment of the antigen-binding molecule or the present invention the first and second domain form an antigen-binding molecule in a format selected from the group consisting of (scFv)₂, scFv-single domain mAb, diabody and oligomers of any of these formats.

According to a particularly preferred embodiment, and as documented in the appended examples, the first and the second domain of the antigen-binding molecule of the invention is a “bispecific single chain antigen-binding molecule”, more preferably a bispecific “single chain Fv” (scFv). Although the two domains of the Fv fragment, VL and VH, are coded for by separate genes, they can be joined, using recombinant methods, by a synthetic linker—as described hereinbefore—that enables them to be made as a single protein chain in which the VL and VH regions pair to form a monovalent molecule; see e.g., Huston et al. (1988) Proc. Natl. Acad. Sci USA 85:5879-5883). These antibody fragments are obtained using conventional techniques known to those with skill in the art, and the fragments are evaluated for function in the same manner as are whole or full-length antibodies. A single-chain variable fragment (scFv) is hence a fusion protein of the variable region of the heavy chain (VH) and of the light chain (VL) of immunoglobulins, usually connected with a short linker peptide as described herein. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and introduction of the linker.

Bispecific single chain antigen-binding molecules are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Brühl, Immunol., (2001), 166, 2420-2426, Kipriyanov, J. Mol. Biol., (1999), 293, 41-56. Techniques described for the production of single chain antibodies (see, inter alia, U.S. Pat. No. 4,946,778, Kontermann and Dübel (2010), loc. cit. and Little (2009), loc. cit.) can be adapted to produce single chain antigen-binding molecules specifically recognizing (an) elected target(s).

Bivalent (also called divalent) or bispecific single-chain variable fragments (bi-scFvs or di-scFvs having the format (scFv)₂ can be engineered by linking two scFv molecules (e.g. with linkers as described hereinbefore). If these two scFv molecules have the same binding specificity, the resulting (scFv)₂ molecule will preferably be called bivalent (i.e. it has two valences for the same target epitope). If the two scFv molecules have different binding specificities, the resulting (scFv)₂ molecule will preferably be called bispecific. The linking can be done by producing a single peptide chain with two VH regions and two VL regions, yielding tandem scFvs (see e.g. Kufer P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. This type is known as diabodies (see e.g. Hollinger, Philipp et al., (July 1993) Proceedings of the National Academy of Sciences of the United States of America 90 (14): 6444-8).

In line with this invention either the first, the second or the first and the second domain may comprise a single domain antibody, respectively the variable domain or at least the CDRs of a single domain antibody. Single domain antibodies comprise merely one (monomeric) antibody variable domain which is able to bind selectively to a specific antigen, independently of other V regions or domains. The first single domain antibodies were engineered from heavy chain antibodies found in camelids, and these are called V_{H}H fragments. Cartilaginous fishes also have heavy chain antibodies (IgNAR) from which single domain antibodies called V_{NAR} fragments can be obtained. An alternative approach is to split the dimeric variable domains from common immunoglobulins e.g. from humans or rodents into monomers, hence obtaining VH or VL as a single domain Ab. Although most research into single domain antibodies is currently based on heavy chain variable domains, nanobodies derived from light chains have also been shown to bind specifically to target epitopes. Examples of single domain antibodies are called sdAb, nanobodies or single variable domain antibodies.

A (single domain mAb)₂ is hence a monoclonal antigen-binding molecule composed of (at least) two single domain monoclonal antibodies, which are individually selected from the group comprising V_{H}, V_{L}, V_{H}H and V_{NAR}. The linker is preferably in the form of a peptide linker. Similarly, an “scFv-single domain mAb” is a monoclonal antigen-binding molecule composed of at least one single domain antibody as described above and one scFv molecule as described above. Again, the linker is preferably in the form of a peptide linker.

Whether or not an antigen-binding molecule competes for binding with another given antigen-binding molecule can be measured in a competition assay such as a competitive ELISA or a cell-based competition assay. Avidin-coupled microparticles (beads) can also be used. Similar to an avidin-coated ELISA plate, when reacted with a biotinylated protein, each of these beads can be used as a substrate on which an assay can be performed. Antigen is coated onto a bead and then precoated with the first antibody. The second antibody is added and any additional binding is determined. Possible means for the read-out includes flow cytometry.

T cells or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (a) and beta (D) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

The CD3 receptor complex is a protein complex and is composed of four chains. In mammals, the complex contains a CD3γ (gamma) chain, a CD36 (delta) chain, and two CD3ε (epsilon) chains. These chains associate with the T cell receptor (TCR) and the so-called ((zeta) chain to form the T cell receptor CD3 complex and to generate an activation signal in T lymphocytes. The CD3γ (gamma), CD36 (delta), and CD3ε (epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3ε gene which resides on chromosome 11. The most preferred epitope of CD3 epsilon is comprised within amino acid residues 1-27 of the human CD3 epsilon extracellular domain. It is envisaged that antigen-binding molecules according to the present invention typically and advantageously show less unspecific T cell activation, which is not desired in specific immunotherapy. This translates to a reduced risk of side effects.

The redirected lysis of target cells via the recruitment of T cells by a multispecific, at least bispecific, antigen-binding molecule involves cytolytic synapse formation and delivery of perforin and granzymes. The engaged T cells are capable of serial target cell lysis, and are not affected by immune escape mechanisms interfering with peptide antigen processing and presentation, or clonal T cell differentiation; see, for example, WO 2007/042261.

Cytotoxicity mediated by antigen-binding molecules of the invention can be measured in various ways. Effector cells can be e.g. stimulated enriched (human) CD8 positive T cells or unstimulated (human) peripheral blood mononuclear cells (PBMC). If the target cells are of macaque origin or express or are transfected with macaque CS1, BCMA, CD20, CD22, FLT3, CD123, CLL1, MSLN, or EpCAM which is bound by the first domain, the effector cells should also be of macaque origin such as a macaque T cell line, e.g. 4119LnPx. The target cells should express (at least the extracellular domain of) CS1, BCMA, CD20, CD22, FLT3, CD123, CLL1, MSLN, or EpCAM, e.g. human or macaque CS1, BCMA, CD20, CD22, FLT3, CD123, CLL1, MSLN, or EpCAM. Target cells can be a cell line (such as CHO) which is stably or transiently transfected with CS1, BCMA, CD20, CD22, FLT3, CD123, CLL1, MSLN, or EpCAM, e.g. human or macaque CS1, BCMA, CD20, CD22, FLT3, CD123, CLL1, MSLN, or EpCAM. Usually EC₅₀ values are expected to be lower with target cell lines expressing higher levels of CS1, BCMA, CD20, CD22, FLT3, CD123, CLL1, MSLN, or EpCAM on the cell surface. The effector to target cell (E:T) ratio is usually about 10:1, but can also vary. Cytotoxic activity of CS1, BCMA, CD20, CD22, FLT3, CD123, CLL1, MSLN, or EpCAMbispecific antigen-binding molecules can be measured in a ⁵¹Cr-release assay (incubation time of about 18 hours) or in a in a FACS-based cytotoxicity assay (incubation time of about 48 hours). Modifications of the assay incubation time (cytotoxic reaction) are also possible. Other methods of measuring cytotoxicity are well-known to the skilled person and comprise MTT or MTS assays, ATP-based assays including bioluminescent assays, the sulforhodamine B (SRB) assay, WST assay, clonogenic assay and the ECIS technology.

The cytotoxic activity mediated by CS1, BCMA, CD20, CD22, FLT3, CD123, CLL1, MSLN, or EpCAMxCD3 bispecific antigen-binding molecules of the present invention is preferably measured in a cell-based cytotoxicity assay. It may also be measured in a ⁵¹Cr-release assay. It is represented by the EC₅₀ value, which corresponds to the half maximal effective concentration (concentration of the antigen-binding molecule which induces a cytotoxic response halfway between the baseline and maximum). Preferably, the EC₅₀ value of the CS1, BCMA, CD20, CD22, FLT3, CD123, CLL1, MSLN, or EpCAMxCD3 bispecific antigen-binding molecules is ≤5000 pM or ≤4000 pM, more preferably ≤3000 pM or ≤2000 pM, even more preferably ≤1000 pM or ≤500 pM, even more preferably ≤400 pM or ≤300 pM, even more preferably ≤200 pM, even more preferably ≤100 pM, even more preferably ≤50 pM, even more preferably ≤20 pM or ≤10 pM, and most preferably 5 pM.

The above given EC₅₀ values can be measured in different assays. The skilled person is aware that an EC₅₀ value can be expected to be lower when stimulated/enriched CD8⁺ T cells are used as effector cells, compared with unstimulated PBMC. It can furthermore be expected that the EC₅₀ values are lower when the target cells express a high number of CS1, BCMA, CD20, CD22, FLT3, CD123, CLL1, MSLN, or EpCAM compared with a low target expression rat. For example, when stimulated/enriched human CD8⁺ T cells are used as effector cells (and either CS1, BCMA, CD20, CD22, FLT3, CD123, CLL1, MSLN, or EpCAM transfected cells such as CHO cells or CS1, BCMA, CD20, CD22, FLT3, CD123, CLL1, MSLN, or EpCAM positive human cell lines are used as target cells), the EC₅₀ value of the CS1, BCMA, CD20, CD22, FLT3, CD123, CLL1, MSLN, or EpCAMxCD3 bispecific antigen-binding molecule is preferably ≤1000 pM, more preferably ≤500 pM, even more preferably ≤250 pM, even more preferably ≤100 pM, even more preferably ≤50 pM, even more preferably ≤10 pM, and most preferably ≤5 pM. When human PBMCs are used as effector cells, the EC₅₀ value of the CS1, BCMA, CD20, CD22, FLT3, CD123, CLL1, MSLN, or EpCAMxCD3 bispecific antigen-binding molecule is preferably ≤5000 pM or ≤4000 pM (in particular when the target cells are CS1, BCMA, CD20, CD22, FLT3, CD123, CLL1, MSLN, or EpCAM positive human cell lines), more preferably ≤2000 pM (in particular when the target cells are CS1, BCMA, CD20, CD22, FLT3, CD123, CLL1, MSLN, or EpCAM transfected cells such as CHO cells), more preferably 1000 pM or ≤500 pM, even more preferably ≤200 pM, even more preferably ≤150 pM, even more preferably ≤100 pM, and most preferably ≤50 pM, or lower. When a macaque T cell line such as LnPx4l 19 is used as effector cells, and a macaque CS1, BCMA, CD20, CD22, FLT3, CD123, CLL1, MSLN, or EpCAM transfected cell line such as CHO cells is used as target cell line, the EC₅₀ value of the CS1, BCMA, CD20, CD22, FLT3, CD123, CLL1, MSLN, or EpCAMxCD3 bispecific antigen-binding molecule is preferably ≤2000 pM or ≤1500 pM, more preferably ≤1000 pM or ≤500 pM, even more preferably ≤300 pM or ≤250 pM, even more preferably ≤100 pM, and most preferably ≤50 pM.

Preferably, the CS1, BCMA, CD20, CD22, FLT3, CD123, CLL1, MSLN, or EpCAMxCD3 bispecific antigen-binding molecules of the present invention do not induce/mediate lysis or do not essentially induce/mediate lysis of CS1, BCMA, CD20, CD22, FLT3, CD123, CLL1, MSLN, or EpCAM negative cells such as CHO cells. The term “do not induce lysis”, “do not essentially induce lysis”, “do not mediate lysis” or “do not essentially mediate lysis” means that an antigen-binding molecule of the present invention does not induce or mediate lysis of more than 30%, preferably not more than 20%, more preferably not more than 10%, particularly preferably not more than 9%, 8%, 7%, 6% or 5% of CS1, BCMA, CD20, CD22, FLT3, CD123, CLL1, MSLN, or EpCAM negative cells, whereby lysis of a CS1, BCMA, CD20, CD22, FLT3, CD123, CLL1, MSLN, or EpCAM positive human cell line is set to be 100%. This usually applies for concentrations of the antigen-binding molecule of up to 500 nM. The skilled person knows how to measure cell lysis without further ado. Moreover, the present specification teaches specific instructions how to measure cell lysis.

The difference in cytotoxic activity between the monomeric and the dimeric isoform of individual CS1, BCMA, CD20, CD22, FLT3, CD123, CLL1, MSLN, or EpCAMxCD3 bispecific antigen-binding molecules is referred to as “potency gap”. This potency gap can e.g. be calculated as ratio between EC₅₀ values of the molecule's monomeric and dimeric form. Potency gaps of the CS1, BCMA, CD20, CD22, FLT3, CD123, CLL1, MSLN, or EpCAMxCD3 bispecific antigen-binding molecules of the present invention are preferably ≤5, more preferably ≤4, even more preferably ≤3, even more preferably ≤2 and most preferably ≤1.

The first and/or the second (or any further) binding domain(s) of the antigen-binding molecule of the invention is/are preferably cross-species specific for members of the mammalian order of primates. Cross-species specific CD3 binding domains are, for example, described in WO 2008/119567. According to one embodiment, the first and/or second binding domain, in addition to binding to human CS1, BCMA, CD20, CD22, FLT3, CD123, CLL1, MSLN, or EpCAM and human CD3, respectively, will also bind to CS1, BCMA, CD20, CD22, FLT3, CD123, CLL1, MSLN, or EpCAM/CD3 of primates including (but not limited to) new world primates (such as Callithrix jacchus, Saguinus Oedipus or Saimiri sciureus), old world primates (such baboons and macaques), gibbons, and non-human homininae.

In one embodiment of the antigen-binding molecule of the invention the first domain binds to human CS1, BCMA, CD20, CD22, FLT3, CD123, CLL1, MSLN, or EpCAM and further binds to macaque CS1, BCMA, CD20, CD22, FLT3, CD123, CLL1, MSLN, or EpCAM, such as CS1, BCMA, CD20, CD22, FLT3, CD123, CLL1, MSLN, or EpCAM ofMacacafascicularis, and more preferably, to macaque CS1, BCMA, CD20, CD22, FLT3, CD123, CLL1, MSLN, or EpCAM expressed on the surface of cells, e.g. such as CHO or 293 cells. The affinity of the first domain for CS1, BCMA, CD20, CD22, FLT3, CD123, CLL1, MSLN, or EpCAM, preferably for human CS1, BCMA, CD20, CD22, FLT3, CD123, CLL1, MSLN, or EpCAM, is preferably ≤100 nM or ≤50 nM, more preferably ≤25 nM or ≤20 nM, more preferably ≤15 nM or ≤10 nM, even more preferably ≤5 nM, even more preferably ≤2.5 nM or ≤2 nM, even more preferably ≤1 nM, even more preferably ≤0.6 nM, even more preferably ≤0.5 nM, and most preferably ≤0.4 nM. The affinity can be measured for example in a BIAcore assay or in a Scatchard assay. Other methods of determining the affinity are also well-known to the skilled person. The affinity of the first domain for macaque CS1, BCMA, CD20, CD22, FLT3, CD123, CLL1, MSLN, or EpCAM is preferably ≤15 nM, more preferably ≤10 nM, even more preferably ≤5 nM, even more preferably ≤1 nM, even more preferably ≤0.5 nM, even more preferably ≤0.1 nM, and most preferably ≤0.05 nM or even ≤0.01 nM.

Preferably the affinity gap of the antigen-binding molecules according to the invention for binding macaque CS1, BCMA, CD20, CD22, FLT3, CD123, CLL1, MSLN, or EpCAM versus human CS1, BCMA, CD20, CD22, FLT3, CD123, CLL1, MSLN, or EpCAM [ma CS1, BCMA, CD20, CD22, FLT3, CD123, CLL1, MSLN, or EpCAM: hu CS1, BCMA, CD20, CD22, FLT3, CD123, CLL1, MSLN, or EpCAM] (as determined e.g. by BiaCore or by Scatchard analysis) is <100, preferably <20, more preferably <15, further preferably <10, even more preferably <8, more preferably <6 and most preferably <2. Preferred ranges for the affinity gap of the antigen-binding molecules according to the invention for binding macaque CS1, BCMA, CD20, CD22, FLT3, CD123, CLL1, MSLN, or EpCAM versus human CS1, BCMA, CD20, CD22, FLT3, CD123, CLL1, MSLN, or EpCAM are between 0.1 and 20, more preferably between 0.2 and 10, even more preferably between 0.3 and 6, even more preferably between 0.5 and 3 or between 0.5 and 2.5, and most preferably between 0.5 and 2 or between 0.6 and 2.

The third binding domain of the antigen-binding molecule of the invention binds to human CD3 epsilon and/or to Macaca CD3 epsilon. In a preferred embodiment the second domain further binds to Callithrix jacchus, Saguinus Oedipus or Saimiri sciureus CD3 epsilon. Callithrix jacchus and Saguinus oedipus are both new world primate belonging to the family of Callitrichidae, while Saimiri sciureus is a new world primate belonging to the family of Cebidae. Said binding domain may preferably be referred to in Table 5 as “12C” or “12C0”.

It is preferred for the antigen-binding molecule of the present invention that the third binding domain which binds to an extracellular epitope of the human and/or the Macaca CD3 epsilon chain comprises a VL region comprising CDR-L1, CDR-L2 and CDR-L3 selected from:

(a) CDR-L1 as depicted in SEQ ID NO: 27 of WO 2008/119567, CDR-L2 as depicted in SEQ ID NO: 28 of WO 2008/119567 and CDR-L3 as depicted in SEQ ID NO: 29 of WO 2008/119567;
(b) CDR-L1 as depicted in SEQ ID NO: 117 of WO 2008/119567, CDR-L2 as depicted in SEQ ID NO: 118 of WO 2008/119567 and CDR-L3 as depicted in SEQ ID NO: 119 of WO 2008/119567; and
(c) CDR-L1 as depicted in SEQ ID NO: 153 of WO 2008/119567, CDR-L2 as depicted in SEQ ID NO: 154 of WO 2008/119567 and CDR-L3 as depicted in SEQ ID NO: 155 of WO 2008/119567.

In a furthermore preferred embodiment of the antigen-binding molecule of the present invention, the third domain which binds to an extracellular epitope of the human and/or the Macaca CD3 epsilon chain comprises a VH region comprising CDR-H1, CDR-H2 and CDR-H3 selected from:

(a) CDR-H1 as depicted in SEQ ID NO: 12 of WO 2008/119567, CDR-H2 as depicted in SEQ ID NO: 13 of WO 2008/119567 and CDR-H3 as depicted in SEQ ID NO: 14 of WO 2008/119567;
(b) CDR-H1 as depicted in SEQ ID NO: 30 of WO 2008/119567, CDR-H2 as depicted in SEQ ID NO: 31 of WO 2008/119567 and CDR-H3 as depicted in SEQ ID NO: 32 of WO 2008/119567;
(c) CDR-H1 as depicted in SEQ ID NO: 48 of WO 2008/119567, CDR-H2 as depicted in SEQ ID NO: 49 of WO 2008/119567 and CDR-H3 as depicted in SEQ ID NO: 50 of WO 2008/119567;
(d) CDR-H1 as depicted in SEQ ID NO: 66 of WO 2008/119567, CDR-H2 as depicted in SEQ ID NO: 67 of WO 2008/119567 and CDR-H3 as depicted in SEQ ID NO: 68 of WO 2008/119567;
(e) CDR-H1 as depicted in SEQ ID NO: 84 of WO 2008/119567, CDR-H2 as depicted in SEQ ID NO: 85 of WO 2008/119567 and CDR-H3 as depicted in SEQ ID NO: 86 of WO 2008/119567;
(f) CDR-H1 as depicted in SEQ ID NO: 102 of WO 2008/119567, CDR-H2 as depicted in SEQ ID NO: 103 of WO 2008/119567 and CDR-H3 as depicted in SEQ ID NO: 104 of WO 2008/119567;
(g) CDR-H1 as depicted in SEQ ID NO: 120 of WO 2008/119567, CDR-H2 as depicted in SEQ ID NO: 121 of WO 2008/119567 and CDR-H3 as depicted in SEQ ID NO: 122 of WO 2008/119567;
(h) CDR-H1 as depicted in SEQ ID NO: 138 of WO 2008/119567, CDR-H2 as depicted in SEQ ID NO: 139 of WO 2008/119567 and CDR-H3 as depicted in SEQ ID NO: 140 of WO 2008/119567;
(i) CDR-H1 as depicted in SEQ ID NO: 156 of WO 2008/119567, CDR-H2 as depicted in SEQ ID NO: 157 of WO 2008/119567 and CDR-H3 as depicted in SEQ ID NO: 158 of WO 2008/119567; and
(j) CDR-H1 as depicted in SEQ ID NO: 174 of WO 2008/119567, CDR-H2 as depicted in SEQ ID NO: 175 of WO 2008/119567 and CDR-H3 as depicted in SEQ ID NO: 176 of WO 2008/119567.

In a preferred embodiment of the antigen-binding molecule of the invention the above described three groups of VL CDRs are combined with the above described ten groups of VH CDRs within the third binding domain to form (30) groups, each comprising CDR-L 1-3 and CDR-H1-3.

It is preferred for the antigen-binding molecule of the present invention that the third domain which binds to CD3 comprises a VL region selected from the group consisting of those depicted in SEQ ID NOs: 17, 21, 35, 39, 53, 57, 71, 75, 89, 93, 107, 111, 125, 129, 143, 147, 161, 165, 179 or 183 of WO 2008/119567 or as depicted in SEQ ID NO: 13 according to the present invention.

It is also preferred that the third domain which binds to CD3 comprises a VH region selected from the group consisting of those depicted in SEQ ID NO: 15, 19, 33, 37, 51, 55, 69, 73, 87, 91, 105, 109, 123, 127, 141, 145, 159, 163, 177 or 181 of WO 2008/119567 or as depicted in SEQ ID NO: 14.

More preferably, the antigen-binding molecule of the present invention is characterized by a third domain which binds to CD3 comprising a VL region and a VH region selected from the group consisting of:

(a) a VL region as depicted in SEQ ID NO: 17 or 21 of WO 2008/119567 and a VH region as depicted in SEQ ID NO: 15 or 19 of WO 2008/119567;
(b) a VL region as depicted in SEQ ID NO: 35 or 39 of WO 2008/119567 and a VH region as depicted in SEQ ID NO: 33 or 37 of WO 2008/119567;
(c) a VL region as depicted in SEQ ID NO: 53 or 57 of WO 2008/119567 and a VH region as depicted in SEQ ID NO: 51 or 55 of WO 2008/119567;
(d) a VL region as depicted in SEQ ID NO: 71 or 75 of WO 2008/119567 and a VH region as depicted in SEQ ID NO: 69 or 73 of WO 2008/119567;
(e) a VL region as depicted in SEQ ID NO: 89 or 93 of WO 2008/119567 and a VH region as depicted in SEQ ID NO: 87 or 91 of WO 2008/119567;
(f) a VL region as depicted in SEQ ID NO: 107 or 111 of WO 2008/119567 and a VH region as depicted in SEQ ID NO: 105 or 109 of WO 2008/119567;
(g) a VL region as depicted in SEQ ID NO: 125 or 129 of WO 2008/119567 and a VH region as depicted in SEQ ID NO: 123 or 127 of WO 2008/119567;
(h) a VL region as depicted in SEQ ID NO: 143 or 147 of WO 2008/119567 and a VH region as depicted in SEQ ID NO: 141 or 145 of WO 2008/119567;
(i) a VL region as depicted in SEQ ID NO: 161 or 165 of WO 2008/119567 and a VH region as depicted in SEQ ID NO: 159 or 163 of WO 2008/119567; and
(j) a VL region as depicted in SEQ ID NO: 179 or 183 of WO 2008/119567 and a VH region as depicted in SEQ ID NO: 177 or 181 of WO 2008/119567.

Also preferred in connection with the antigen-binding molecule of the present invention is a third domain which binds to CD3 comprising a VL region as depicted in SEQ ID NO: 13 and a VH region as depicted in SEQ ID NO: 14.

According to a preferred embodiment of the antigen-binding molecule of the present invention, the first and/or the third domain have the following format: The pairs of VH regions and VL regions are in the format of a single chain antibody (scFv). The VH and VL regions are arranged in the order VH-VL or VL-VH. It is preferred that the VH-region is positioned N-terminally of a linker sequence, and the VL-region is positioned C-terminally of the linker sequence.

A preferred embodiment of the above described antigen-binding molecule of the present invention is characterized by the third domain which binds to CD3 comprising an amino acid sequence selected from the group consisting of SEQ ID NOs: 23, 25, 41, 43, 59, 61, 77, 79, 95, 97, 113, 115, 131, 133, 149, 151, 167, 169, 185 or 187 of WO 2008/119567 or as depicted in SEQ ID NO: 15.

The invention further provides an antigen-binding molecule comprising or having an amino acid sequence (full bispecific antigen-binding molecule) selected from the group consisting of any of 673, 676, 679, 682, 685, 688, 691, 694, 697, 700, 703, 706, 709, 712, 715, 718, 721, 724, 727, 730, 733, 736, 739, 742, 745, 748, 751, 754, 757, 760, 763, 766, 769, 772, 775, 778, 781, 784, 787, 790, 793, 796, 799, 802, 805, 808, 811, 814, 817, 820, 823, 826, 829, 832, 835, 838, 841, 844, 847, 850, 853, 856, 859, 862, 865, 868, 871, 1437, 1440, 1443, 1446, 1449, 1452, 1455, 1458, 1461, 1464, 1467, 1470, 1473, 1476, 1479, 1482, 1485, 1488, 1499, 1667, 1670, 1673, 1676, 1679, 1682, 1685, 1688, 1691, 1694, 1697, 1700, 1703, 1706, 1709, 1712, 1715, 1718, 1721, 1724, 1727, 1730, 1733, 1736, 1739, 1742, 1745, 1748, 1751, 1754, 1757, 1760, 1763, 1766, 1769, 1772, 1775, 1778, 1781, 1784, 1787, 1790, 1793, 1796, 1799, 1802, 1805, 1808, 1811, 1814, 1817, 1820, 1823, 1826, 1829, 1838, 1851, 1864, 1877, 1890, 1903, 1916, 1933, 1946, 1959, 1972, 1985, 1998, 2011, 2024, 2037, 2050, 2063, 2076, 2089, 2102, 2115, 2128, 2141, 2154, 2167, 2180, 2194, 2206, 2219, 2232, 2245, 2258, 2262, 2270, 2271, 2280, 2281, 2290, 2291, 2300, 2301, 2310, 2311, 2320, 2321, 2330, 2331, 2340, 2341, 2350, 2351, 2360, 2361, 2370, 2371, 2380, 2381, 2390, 2391, 2400, 2401, 2410, 2411, 2420, 2421, 2430, 2431, 2440, 2441, 2450, 2451, 2460, 2461, 2470, 2471, 2480, 2481, 2490, 2491, 2500, 2501, 2510, 2511, 2520, 2521, 2530, 2531, 2540, 2541, 2550, 2551, 2560, 2561, 2570, 2571, 2580, 2581, 2590, 2591, 2600, 2601, 2610, 2611, 2620, 2621, 2630, 2631, 2640, 2641, 2650, 2651, 2660, 2661, 2670, 2671, 2680, 2681, 2690, 2691, 2700, 2701, 2710, 2711, 2720, 2721, 2730, 2731, 2740, 2741, 2750, 2751, 2760, 2761, 2770, 2771, 2780, 2781, 2790, 2791, 2800, 2801, 2810, 2811, 2820, 2821, 2830, 2831, 2840, 2841, 2850, 2851, 2860, 2861, 2870, 2871, 2880, 2881, 2890, 2891, 2900, 2901, 2910, 2911, 2920, 2921, 2930, 2931, 2940, 2941, 2950, 2951, 2960, 2961, 2970, 2971, 2980, 2981, 2990, 2991, 3000, 3001, 3010, 3011, 3020, 3021, 3030, 3031, 3040, 3041, 3050, 3051, 3060, 3061, 3070, 3071, 3080, 3081, 3090, 3091, 3100, 3101, 3110, 3111, 3120, 3121, 3130, 3131, 3140, 3141, 3150, 3151, 3160, 3161, 3170, 3171, 3180, 3181, 3190, 3191, 3200, 3201, 3210, 3211, 3220, 3221, 3231, 3240, 3241, 3250, 3251, 3260, 3261, 3270, 3271, 3280, 3281, 3290, 3291, 3300, 3301, 3310, 3311, 3320, 3321, 3330, 3331, 3340, 3341, 3344, 3345, 3356, 3367, 3378, 3389, 3400, 3411, 3422, 3433, 3444, 3455, 3466, 3477, 3488, 3499, 3510, 3521, 3532, 3543, 3554, 3565, 3576, 3579, 382, 3585, 3588, 3591, 3594, 3597, 3600, 3603, 3606, 3609, 3612, 3615, 3618, 3621, 3624, 3627, 3630, 3633, 3636, 3639, 3642, 3645, 3648, 3651, 3654, 3657, 3660, 3663, 3666, 3669, 3672, 3675, 3678, 3689, 3700, 3704, 3705, 3708, 3709, 3710, 3711, 3722, 3733, 3736, 3739, 3744, 3747, 3748, 3756, 3757, 3761, and 3762, preferably 1437, or having an amino acid sequence having at least 90, 91, 92, 93, 94 95, 96, 97, 98 or 99% identity to said sequences.

Covalent modifications of the antigen-binding molecules are also included within the scope of this invention, and are generally, but not always, done post-translationally. For example, several types of covalent modifications of the antigen-binding molecule are introduced into the molecule by reacting specific amino acid residues of the antigen-binding molecule with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or 1311 to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking the antigen-binding molecules of the present invention to a water-insoluble support matrix or surface for use in a variety of methods. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates as described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the antigen-binding molecules included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antigen-binding molecule is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the amino acid sequence of an antigen-binding molecule is preferably altered through changes at the DNA level, particularly by mutating the DNA encoding the polypeptide at preselected bases such that codons are generated that will translate into the desired amino acids.

Another means of increasing the number of carbohydrate moieties on the antigen-binding molecule is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting antigen-binding molecule may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Other modifications of the antigen-binding molecule are also contemplated herein. For example, another type of covalent modification of the antigen-binding molecule comprises linking the antigen-binding molecule to various non-proteinaceous polymers, including, but not limited to, various polyols such as polyethylene glycol, polypropylene glycol, polyoxyalkylenes, or copolymers of polyethylene glycol and polypropylene glycol, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the antigen-binding molecule, e.g. in order to facilitate the addition of polymers such as PEG.

In some embodiments, the covalent modification of the antigen-binding molecules of the invention comprises the addition of one or more labels. The labelling group may be coupled to the antigen-binding molecule via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to:
a) isotopic labels, which may be radioactive or heavy isotopes, such as radioisotopes or radionuclides (e.g., ³H, ⁴C, ¹⁵N, ³⁵S, ⁸⁹Zr, ⁹⁰Y, ⁹⁹Tc, ¹¹¹In, ¹²⁵I, ¹³¹I)
b) magnetic labels (e.g., magnetic particles)
c) redox active moieties
d) optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluors or proteinaceous fluors
e) enzymatic groups (e.g. horseradish peroxidase, β-galactosidase, luciferase, alkaline phosphatase)
f) biotinylated groups
g) predetermined polypeptide epitopes recognized by a secondary reporter (e.g., leucine zipper pair sequences, binding sides for secondary antibodies, metal binding domains, epitope tags, etc.)

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658; 5,418,155; 5,683,888; 5,741,668; 5,777,079; 5,804,387; 5,874,304; 5,876,995; 5,925,558).

The antigen-binding molecule of the invention may also comprise additional domains, which are e.g. helpful in the isolation of the molecule or relate to an adapted pharmacokinetic profile of the molecule. Domains helpful for the isolation of an antigen-binding molecule may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed antigen-binding molecules may comprise a His-tag domain, which is generally known as a repeat of consecutive His residues in the amino acid sequence of a molecule, preferably of five, and more preferably of six His residues (hexa-histidine). The His-tag may be located e.g. at the N- or C-terminus of the antigen-binding molecule, preferably it is located at the C-terminus. Most preferably, a hexa-histidine tag (HHHHHH) (SEQ ID NO:16) is linked via peptide bond to the C-terminus of the antigen-binding molecule according to the invention. Additionally, a conjugate system of PLGA-PEG-PLGA may be combined with a poly-histidine tag for sustained release application and improved pharmacokinetic profile.

Amino acid sequence modifications of the antigen-binding molecules described herein are also contemplated. For example, it may be desirable to improve the binding affinity and/or other biological properties of the antigen-binding molecule. Amino acid sequence variants of the antigen-binding molecules are prepared by introducing appropriate nucleotide changes into the antigen-binding molecules nucleic acid, or by peptide synthesis. All of the below described amino acidacid sequence modifications should result in an antigen-binding molecule which still retains the desired biological activity (binding to CS1, BCMA, CD20, CD22, FLT3, CD123, CLL1, MSLN, or EpCAM and to CD3) of the unmodified parental molecule.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (GIn or Q); glutamic acid (GIu or E); glycine (GIy or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, GIu); a positively charged sidechain (e.g., Arg, His, Lys); or an uncharged polar side chain (e.g., Asn, Cys, GIn, GIy, His, Met, Phe, Ser, Thr, Trp, and Tyr).

Amino acid modifications include, for example, deletions from, and/or insertions into, and/or substitutions of, residues within the amino acid sequences of the antigen-binding molecules. Any combination of deletion, insertion, and substitution is made to arrive at the final construct, provided that the final construct possesses the desired characteristics. The amino acid changes also may alter post-translational processes of the antigen-binding molecules, such as changing the number or position of glycosylation sites.

For example, 1, 2, 3, 4, 5, or 6 amino acids may be inserted, substituted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted, substituted or deleted in each of the FRs. Preferably, amino acid sequence insertions into the antigen-binding molecule include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. Corresponding modifications may also performed within the third domain of the antigen-binding molecule of the invention. An insertional variant of the antigen-binding molecule of the invention includes the fusion to the N-terminus or to the C-terminus of the antigen-binding molecule of an enzyme or the fusion to a polypeptide.

The sites of greatest interest for substitutional mutagenesis include (but are not limited to) the CDRs of the heavy and/or light chain, in particular the hypervariable regions, but FR alterations in the heavy and/or light chain are also contemplated. The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the antigen-binding molecules that are preferred locations for mutagenesis is called “alanine scanning mutagenesis” as described by Cunningham and Wells in Science, 244: 1081-1085 (1989). Here, a residue or group of target residues within the antigen-binding molecule is/are identified (e.g. charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions are then refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed antigen-binding molecule variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of antigen binding activities, such as CS1, BCMA, CD20, CD22, FLT3, CD123, CLL1, MSLN, or EpCAM or CD3 binding.

Generally, if amino acids are substituted in one or more or all of the CDRs of the heavy and/or light chain, it is preferred that the then-obtained “substituted” sequence is at least 60% or 65%, more preferably 70% or 75%, even more preferably 80% or 85%, and particularly preferably 90% or 95% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the antigen-binding molecule may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions” listed in Table 3, below) is envisaged as long as the antigen-binding molecule retains its capability to bind to CS1, BCMA, CD20, CD22, FLT3, CD123, CLL1, MSLN, or EpCAM via the first domain and to CD3 epsilon via the second domain and/or its CDRs have an identity to the then substituted sequence (at least 60% or 65%, more preferably 70% or 75%, even more preferably 80% or 85%, and particularly preferably 90% or 95% identical to the “original” CDR sequence).

Conservative substitutions are shown in Table 3 under the heading of “preferred substitutions”. If such substitutions result in a change in biological activity, then more substantial changes, denominated “exemplary substitutions” in Table 3, or as further described below in reference to amino acid classes, may be introduced and the products screened for a desired characteristic.

TABLE 3
Amino acid substitutions

| Original | Exemplary Substitutions | Preferred Substitutions |
|---|---|---|
| Ala (A) | val, leu, ile | Val |
| Arg (R) | lys, gln, asn | Lys |
| Asn (N) | gin, his, asp, lys, arg | Gln |
| Asp (D) | glu, asn | Glu |
| Cys (C) | ser, ala | ser |
| Gln (Q) | asn, glu | asn |
| Glu (E) | asp, gln | asp |
| Gly (G) | Ala | ala |
| His (H) | asn, gin, lys, arg | arg |
| Ile (I) | leu, val, met, ala, phe | leu |
| Leu (L) | norleucine, ile, val, met, ala | ile |
| Lys (K) | arg, gln, asn | arg |
| Met (M) | leu, phe, ile | leu |
| Phe (F) | leu, val, ile, ala, tyr | tyr |
| Pro (P) | Ala | ala |
| Ser (S) | Thr | thr |
| Thr (T) | Ser | ser |
| Trp (W) | tyr, phe | tyr |
| Tyr (Y) | trp, phe, thr, ser | phe |
| Val (V) | ile, leu, met, phe, ala | leu |

Substantial modifications in the biological properties of the antigen-binding molecule of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr; asn, gln (3) acidic: asp, glu; (4) basic: his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the antigen-binding molecule may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

For amino acid sequences, sequence identity and/or similarity is determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABJOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs or VH/VL sequences are at least 60% to the sequences depicted herein, and more typically with preferably increasing homologies or identities of at least 65% or 70%, more preferably at least 75% or 80%, even more preferably at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the antigen-binding molecule. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs or VH/VL sequences and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Thus, a “variant CDR” or a “variant VH/VL region” is one with the specified homology, similarity, or identity to the parent CDR/VH/VL of the invention, and shares biological function, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR or VH/VL.

In one embodiment, the percentage of identity to human germline of the antigen-binding molecules according to the invention is ≥70% or ≥75%, more preferably ≥80% or ≥85%, even more preferably ≥90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang & Foote (“Immunogenicity of engineered antibodies”; Methods 36 (2005) 3-10) demonstrate that the reduction of non-human portions of drug antigen-binding molecules leads to a decrease of risk to induce anti-drug antibodies in the patients during treatment. By comparing an exhaustive number of clinically evaluated antibody drugs and the respective immunogenicity data, the trend is shown that humanization of the V-regions of antibodies makes the protein less immunogenic (average 5.1% of patients) than antibodies carrying unaltered non-human V regions (average 23.59% of patients). A higher degree of identity to human sequences is hence desirable for V-region based protein therapeutics in the form of antigen-binding molecules. For this purpose of determining the germline identity, the V-regions of VL can be aligned with the amino acid sequences of human germline V segments and J segments (http://vbase.mrc-cpe.cam.ac.uk/) using Vector NTI software and the amino acid sequence calculated by dividing the identical amino acid residues by the total number of amino acid residues of the VL in percent. The same can be for the VH segments (http://vbase.mrc-cpe.cam.ac.uk/) with the exception that the VH CDR3 may be excluded due to its high diversity and a lack of existing human germline VH CDR3 alignment partners. Recombinant techniques can then be used to increase sequence identity to human antibody germline genes.

In a further embodiment, the bispecific antigen-binding molecules of the present invention exhibit high monomer yields under standard research scale conditions, e.g., in a standard two-step purification process. Preferably the monomer yield of the antigen-binding molecules according to the invention is ≥0.25 mg/L supernatant, more preferably ≥0.5 mg/L, even more preferably ≥1 mg/L, and most preferably ≥3 mg/L supernatant.

Likewise, the yield of the dimeric antigen-binding molecule isoforms and hence the monomer percentage (i.e., monomer: (monomer+dimer)) of the antigen-binding molecules can be determined. The productivity of monomeric and dimeric antigen-binding molecules and the calculated monomer percentage can e.g. be obtained in the SEC purification step of culture supernatant from standardized research-scale production in roller bottles. In one embodiment, the monomer percentage of the antigen-binding molecules is ≥80%, more preferably ≥85%, even more preferably ≥90%, and most preferably ≥95%.

In one embodiment, the antigen-binding molecules have a preferred plasma stability (ratio of EC50 with plasma to EC50 w/o plasma) of ≤5 or ≤4, more preferably ≤3.5 or ≤3, even more preferably ≤2.5 or ≤2, and most preferably ≤1.5 or ≤1. The plasma stability of an antigen-binding molecule can be tested by incubation of the construct in human plasma at 37° C. for 24 hours followed by EC50 determination in a ⁵¹chromium release cytotoxicity assay. The effector cells in the cytotoxicity assay can be stimulated enriched human CD8 positive T cells. Target cells can e.g. be CHO cells transfected with human CS1, BCMA, CD20, CD22, FLT3, CD123, CLL1, MSLN, or EpCAM. The effector to target cell (E:T) ratio can be chosen as 10:1 or 5:1. The human plasma pool used for this purpose is derived from the blood of healthy donors collected by EDTA coated syringes. Cellular components are removed by centrifugation and the upper plasma phase is collected and subsequently pooled. As control, antigen-binding molecules are diluted immediately prior to the cytotoxicity assay in RPMI-1640 medium. The plasma stability is calculated as ratio of EC50 (after plasma incubation) to EC50 (control).

It is furthermore preferred that the monomer to dimer conversion of antigen-binding molecules of the invention is low. The conversion can be measured under different conditions and analyzed by high performance size exclusion chromatography. For example, incubation of the monomeric isoforms of the antigen-binding molecules can be carried out for 7 days at 37° C. and concentrations of e.g. 100 μg/ml or 250 μg/ml in an incubator. Under these conditions, it is preferred that the antigen-binding molecules of the invention show a dimer percentage that is ≤5%, more preferably ≤4%, even more preferably ≤3%, even more preferably ≤2.5%, even more preferably ≤2%, even more preferably ≤1.5%, and most preferably ≤1% or ≤0.5% or even 0%.

It is also preferred that the bispecific antigen-binding molecules of the present invention present with very low dimer conversion after a number of freeze/thaw cycles. For example, the antigen-binding molecule monomer is adjusted to a concentration of 250 μg/ml e.g. in generic formulation buffer and subjected to three freeze/thaw cycles (freezing at −80° C. for 30 min followed by thawing for 30 min at room temperature), followed by high performance SEC to determine the percentage of initially monomeric antigen-binding molecule, which had been converted into dimeric antigen-binding molecule. Preferably the dimer percentages of the bispecific antigen-binding molecules are ≤5%, more preferably ≤4%, even more preferably ≤3%, even more preferably ≤2.5%, even more preferably ≤2%, even more preferably ≤1.5%, and most preferably ≤1% or even ≤0.5%, for example after three freeze/thaw cycles.

The bispecific antigen-binding molecules of the present invention preferably show a favorable thermostability with aggregation temperatures ≥45° C. or ≥50° C., more preferably ≥52° C. or ≥54° C., even more preferably ≥56° C. or ≥57° C., and most preferably ≥58° C. or ≥59° C. The thermostability parameter can be determined in terms of antibody aggregation temperature as follows: Antibody solution at a concentration 250 pg/ml is transferred into a single use cuvette and placed in a Dynamic Light Scattering (DLS) device. The sample is heated from 40° C. to 70° C. at a heating rate of 0.5° C./min with constant acquisition of the measured radius. Increase of radius indicating melting of the protein and aggregation is used to calculate the aggregation temperature of the antibody.

Alternatively, temperature melting curves can be determined by Differential Scanning Calorimetry (DSC) to determine intrinsic biophysical protein stabilities of the antigen-binding molecules. These experiments are performed using a MicroCal LLC (Northampton, Mass., USA) VP-DSC device. The energy uptake of a sample containing an antigen-binding molecule is recorded from 20° C. to 90° C. compared to a sample containing only the formulation buffer. The antigen-binding molecules are adjusted to a final concentration of 250 μg/ml e.g. in SEC running buffer. For recording of the respective melting curve, the overall sample temperature is increased stepwise. At each temperature T energy uptake of the sample and the formulation buffer reference is recorded. The difference in energy uptake Cp (kcal/mole/° C.) of the sample minus the reference is plotted against the respective temperature. The melting temperature is defined as the temperature at the first maximum of energy uptake.

The CS1, BCMA, CD20, CD22, FLT3, CD123, CLL1, MSLN, or EpCAMxCD3 bispecific antigen-binding molecules of the invention are also envisaged to have a turbidity (as measured by OD340 after concentration of purified monomeric antigen-binding molecule to 2.5 mg/ml and overnight incubation) of ≤0.2, preferably of ≤0.15, more preferably of ≤0.12, even more preferably of ≤0.1, and most preferably of ≤0.08.

In a further embodiment the antigen-binding molecule according to the invention is stable at physiologic or slightly lower pH, i.e. about pH 7.4 to 6.0. The more tolerant the antigen-binding molecule behaves at unphysiologic pH such as about pH 6.0, the higher is the recovery of the antigen-binding molecule eluted from an ion exchange column relative to the total amount of loaded protein. Recovery of the antigen-binding molecule from an ion (e.g., cation) exchange column at about pH 6.0 is preferably ≥30%, more preferably ≥40%, more preferably ≥50%, even more preferably ≥60%, even more preferably ≥70%, even more preferably ≥80%, even more preferably ≥90%, even more preferably ≥95%, and most preferably ≥99%.

It is furthermore envisaged that the bispecific antigen-binding molecules of the present invention exhibit therapeutic efficacy or anti-tumor activity. This can e.g. be assessed in a study as disclosed in the following generalized example of an advanced stage human tumor xenograft model:

On day 1 of the study, 5×10⁶ cells of a human target cell antigen (here: CS1, BCMA, CD20, CD22, FLT3, CD123, CLL1, MSLN, or EpCAM) positive cancer cell line are subcutaneously injected in the right dorsal flank of female NOD/SCID mice. When the mean tumor volume reaches about 100 mm3, in vitro expanded human CD3 positive T cells are transplanted into the mice by injection of about 2×10⁷ cells into the peritoneal cavity of the animals. Mice of vehicle control group 1 do not receive effector cells and are used as an untransplanted control for comparison with vehicle control group 2 (receiving effector cells) to monitor the impact of T cells alone on tumor growth. The antibody treatment starts when the mean tumor volume reaches about 200 mm³. The mean tumor size of each treatment group on the day of treatment start should not be statistically different from any other group (analysis of variance). Mice are treated with 0.5 mg/kg/day of a CS1, BCMA, CD20, CD22, FLT3, CD123, CLL1, MSLN, or EpCAMxCD3 bispecific antigen-binding molecule by intravenous bolus injection for about 15 to 20 days. Tumors are measured by caliper during the study and progress evaluated by intergroup comparison of tumor volumes (TV). The tumor growth inhibition T/C [%] is determined by calculating TV as T/C %=100×(median TV of analyzed group)/(median TV of control group 2).

The skilled person knows how to modify or adapt certain parameters of this study, such as the number of injected tumor cells, the site of injection, the number of transplanted human T cells, the amount of bispecific antigen-binding molecules to be administered, and the timelines, while still arriving at a meaningful and reproducible result. Preferably, the tumor growth inhibition T/C [%] is ≤70 or ≤60, more preferably ≤50 or ≤40, even more preferably ≤30 or ≤20 and most preferably ≤10 or ≤5 or even ≤2.5. Tumor growth inhibition is preferably close to 100%.

In a preferred embodiment of the antigen-binding molecule of the invention the antigen-binding molecule is a single chain antigen-binding molecule.

Also in a preferred embodiment of the antigen-binding molecule of the invention said third domain comprises in an amino to carboxyl order:
hinge-CH2-CH3-linker-hinge-CH2-CH3.

In one embodiment of the invention each of said polypeptide monomers of the third domain has an amino acid sequence that is at least 90% identical to a sequence selected from the group consisting of: SEQ ID NO: 17-24. In a preferred embodiment or the invention each of said polypeptide monomers has an amino acid sequence selected from SEQ ID NO: 17-24.

Also in one embodiment of the invention the CH2 domain of one or preferably each (both) polypeptide monomers of the third domain comprises an intra domain cysteine disulfide bridge. As known in the art the term “cysteine disulfide bridge” refers to a functional group with the general structure R—S—S—R. The linkage is also called an SS-bond or a disulfide bridge and is derived by the coupling of two thiol groups of cysteine residues. It is particularly preferred for the antigen-binding molecule of the invention that the cysteines forming the cysteine disulfide bridge in the mature antigen-binding molecule are introduced into the amino acid sequence of the CH2 domain corresponding to 309 and 321 (Kabat numbering).

In one embodiment of the invention a glycosylation site in Kabat position 314 of the CH2 domain is removed. It is preferred that this removal of the glycosylation site is achieved by a N314X substitution, wherein X is any amino acid excluding Q. Said substitution is preferably a N314G. In a more preferred embodiment, said CH2 domain additionally comprises the following substitutions (position according to Kabat) V321C and R309C (these substitutions introduce the intra domain cysteine disulfide bridge at Kabat positions 309 and 321).

It is assumed that the preferred features of the antigen-binding molecule of the invention compared e.g. to the bispecific heteroFc antigen-binding molecule known in the art (FIG. F1b) may be inter alia related to the introduction of the above described modifications in the CH2 domain. Thus, it is preferred for the construct of the invention that the CH2 domains in the third domain of the antigen-binding molecule of the invention comprise the intra domain cysteine disulfide bridge at Kabat positions 309 and 321 and/or the glycosylation site at Kabat position 314 is removed, preferably by a N314G substitution.

In a further preferred embodiment of the invention the CH2 domains in the third domain of the antigen-binding molecule of the invention comprise the intra domain cysteine disulfide bridge at Kabat positions 309 and 321 and the glycosylation site at Kabat position 314 is removed by a N314G substitution. Most preferably, the polypeptide monomer of the third domain of the antigen-binding molecule of the invention has an amino acid sequence selected from the group consisting of SEQ ID NO: 17 and 18.

In one embodiment the invention provides an antigen-binding molecule, wherein:
(i) the first domain comprises two antibody variable domains and the second domain comprises two antibody variable domains;
(ii) the first domain comprises one antibody variable domain and the second domain comprises two antibody variable domains;
(iii) the first domain comprises two antibody variable domains and the second domain comprises one antibody variable domain; or
(iv) the first domain comprises one antibody variable domain and the second domain comprises one antibody variable domain.

Accordingly, the first and the second domain may be binding domains comprising each two antibody variable domains such as a VH and a VL domain. Examples for such binding domains comprising two antibody variable domains where described herein above and comprise e.g. Fv fragments, scFv fragments or Fab fragments described herein above. Alternatively either one or both of those binding domains may comprise only a single variable domain. Examples for such single domain binding domains where described herein above and comprise e.g. nanobodies or single variable domain antibodies comprising merely one variable domain, which may be VHH, VH or VL, that specifically bind an antigen or epitope independently of other V regions or domains.

In a preferred embodiment of the antigen-binding molecule of the invention first and second domain are fused to the third domain via a peptide linker. Preferred peptide linker have been described herein above and are characterized by the amino acid sequence Gly-Gly-Gly-Gly-Ser, i.e. Gly₄Ser (SEQ ID NO: 1), or polymers thereof, i.e. (Gly₄Ser)x, where x is an integer of 1 or greater (e.g. 2 or 3). A particularly preferred linker for the fusion of the first and second domain to the third domain is depicted in SEQ ID NO: 1.

In a preferred embodiment the antigen-binding molecule of the invention is characterized to comprise in an amino to carboxyl order:
(a) the first domain;
(b) a peptide linker having an amino acid sequence selected from the group consisting of SEQ ID NO: 1-3;
(c) the second domain;
(d) a peptide linker having an amino acid sequence selected from the group consisting of SEQ ID NO: 1, 2, 3, 9, 10, 11 and 12;
(e) the first polypeptide monomer of the third domain;
(f) a peptide linker having an amino acid sequence selected from the group consisting of SEQ ID NO: 5, 6, 7 and 8; and
(g) the second polypeptide monomer of the third domain.

The antigen-binding molecule of the present invention comprises a first domain which binds to CS1, BCMA, CD20, CD22, FLT3, CD123, CLL1, MSLN, or EpCAM, preferably to the extracellular domain(s) (ECD) of CS1, BCMA, CD20, CD22, FLT3, CD123, CLL1, MSLN, or EpCAM. It is understood that the term “binding to the extracellular domain of CS1, BCMA, CD20, CD22, FLT3, CD123, CLL1, MSLN, or EpCAM”, in the context of the present invention, implies that the binding domain binds to CS1, BCMA, CD20, CD22, FLT3, CD123, CLL1, MSLN, or EpCAM expressed on the surface of a target cell. The first domain according to the invention hence preferably binds to CS1, BCMA, CD20, CD22, FLT3, CD123, CLL1, MSLN, or EpCAM when it is expressed by naturally expressing cells or cell lines, and/or by cells or cell lines transformed or (stably/transiently) transfected with CS1, BCMA, CD20, CD22, FLT3, CD123, CLL1, MSLN, or EpCAM. In a preferred embodiment the first binding domain also binds to CS1, BCMA, CD20, CD22, FLT3, CD123, CLL1, MSLN, or EpCAM when CS1, BCMA, CD20, CD22, FLT3, CD123, CLL1, MSLN, or EpCAM is used as a “target” or “ligand” molecule in an in vitro binding assay such as BIAcore or Scatchard. The “target cell” can be any prokaryotic or eukaryotic cell expressing CS1, BCMA, CD20, CD22, FLT3, CD123, CLL1, MSLN, or EpCAM on its surface; preferably the target cell is a cell that is part of the human or animal body, such as a specific CS1, BCMA, CD20, CD22, FLT3, CD123, CLL1, MSLN, or EpCAM expressing cancer or tumor cell.

Preferably, the first binding domain binds to human CS1, BCMA, CD20, CD22, FLT3, CD123, CLL1, MSLN, or EpCAM/CS1, BCMA, CD20, CD22, FLT3, CD123, CLL1, MSLN, or EpCAM ECD. In a further preferred embodiment, it binds to macaque CS1, BCMA, CD20, CD22, FLT3, CD123, CLL1, MSLN, or EpCAM/CS1, BCMA, CD20, CD22, FLT3, CD123, CLL1, MSLN, or EpCAM ECD. According to the most preferred embodiment, it binds to both the human and the macaque CS1, BCMA, CD20, CD22, FLT3, CD123, CLL1, MSLN, or EpCAM/CS1, BCMA, CD20, CD22, FLT3, CD123, CLL1, MSLN, or EpCAM ECD. The “CS1, BCMA, CD20, CD22, FLT3, CD123, CLL1, MSLN, or EpCAM extracellular domain” or “CS1, BCMA, CD20, CD22, FLT3, CD123, CLL1, MSLN, or EpCAM ECD” refers to the CS1, BCMA, CD20, CD22, FLT3, CD123, CLL1, MSLN, or EpCAM region or sequence which is essentially free of transmembrane and cytoplasmic domains of CS1, BCMA, CD20, CD22, FLT3, CD123, CLL1, MSLN, or EpCAM. It will be understood by the skilled artisan that the transmembrane domain identified for the CS1, BCMA, CD20, CD22, FLT3, CD123, CLL1, MSLN, or EpCAM polypeptide of the present invention is identified pursuant to criteria routinely employed in the art for identifying that type of hydrophobic domain. The exact boundaries of a transmembrane domain may vary but most likely by no more than about 5 amino acids at either end of the domain specifically mentioned herein.

Preferred binding domains which bind to CD3 are disclosed in WO 2010/037836, and WO 2011/121110. Any binding domain for CD3 described in these applications may be used in the context of the present invention.

In line with an embodiment of the present invention, multispecific antibody comprise a first and/or a second binding domain comprising a VH region comprising CDR-H1, CDR-H2 and CDR-H3 selected from CDR-H1, CDR-H2 and CDR-H3 selected from the group consisting of SEQ ID Nos: for CD123 33 to 35, 44 to 46, 55 to 57, 66 to 68, 77 to 79, 88 to 90, 99 to 101, 110 to 112, 121 to 123, 132 to 134, 143 to 145, 154 to 156, 165 to 167, 176 to 178, 187 to 189, 198 to 200,209 to 211,220 to 222, 231 to 233, 242 to 244, 253 to 255, 264 to 266, 275 to 277, 286 to 288, 297 to 299, 308 to 310, 319 to 321, 330 to 332, 341 to 343, 352 to 354, 363 to 365, 374 to 376, 385 to 387, 396 to 398, 407 to 409, 418 to 420, 429 to 431, 440 to 442, 451 to 453, 462 to 464, 473 to 475, 484 to 486, 495 to 497, 506 to 508, 517 to 519, 528 to 530, 539 to 541, 550 to 552, 561 to 563, 572 to 574, 583 to 585, 594 to 596, 605 to 607, 616 to 618, 627 to 629, for FLT3: 638 to 640, 649 to 651, 660 to 662, for CS1: 896 to 898, 907 to 909, 918 to 920, 929 to 931, 940 to 942, 951 to 953, 962 to 964, 973 to 975, 984 to 986, 995 to 997, 1006 to 1008, 1017 to 1019, 1028 to 1030, 1039 to 1041, 1050 to 1052, 1061 to 1063, 1072 to 1074, 1083 to 1085, 1094 to 1096, 1105 to 1107, 1116 to 1118, 1127 to 1129, 1138 to 1140, 1149 to 1151, 1160 to 1162, 1171 to 1173, 1182 to 1184, 1193 to 1195, 1204 to 1206, 1215 to 1217, 1226 to 1228, 1237 to 1239, 1248 to 1250, 1259 to 1261, 1270 to 1272, 1281 to 1283, 1292 to 1294, 1303 to 1305, 1314 to 1316, 1325 to 1327, 1336 to 1338, 1347 to 1349, 1358 to 1360, 1369 to 1371, 1380 to 1382, 1391 to 1393, for BCMA: 1402 to 1404, 1413 to 1415, 1424 to 1426, for CD22: 1489 to 1491, 1500 to 1502, 1511 to 1513, 1522 to 1524, 1533 to 1535, 1544 to 1546, for CD20: 1555 to 1557, 1566 to 1568, 1577 to 1579, 1588 to 1590, 1599 to 1601, 1610 to 1612, 1621 to 1623, 1632 to 1634, 1643 to 1645, and 1654 to 1656.

In line with an embodiment of the present invention, multispecific antibody comprise a first and/or a second binding domain comprising a VL region comprising CDR-L1, CDR-L2 and CDR-L3 selected from CDR-L1, CDR-L2 and CDR-L3 selected from the group consisting of SEQ ID Nos: for CD123: 36 to 38, 47 to 49, 58 to 60, 69 to 71, 80 to 82, 91 to 93, 102 to 104, 113 to 115, 124 to 126, 135 to 137, 146 to 148, 157 to 159, 168 to 170, 179 to 181, 190 to 192,201 to 203,212 to 214,223 to 225, 234 to 236, 245 to 247, 256 to 258, 267 to 269, 278 to 280, 289 to 291, 300 to 302, 311 to 313, 322 to 324, 333 to 335, 344 to 346, 355 to 357, 366 to 368, 377 to 379, 388 to 390, 399 to 401, 410 to 412, 421 to 423, 432 to 434, 443 to 445, 454 to 456, 465 to 467, 476 to 478, 487 to 489, 498 to 500, 509 to 511, 520 to 522, 531 to 533, 542 to 544, 553 to 555, 564 to 566, 575 to 577, 586 to 588, 597 to 599, 608 to 610, 619 to 621, 630 to 632, for FLT3: 641 to 643, 652 to 654, 663 to 665, for CS1: 899 to 901, 910 to 912, 921 to 923, 932 to 934, 943 to 945, 954 to 956, 965 to 967, 976 to 978, 987 to 989, 998 to 1000, 1009 to 1011, 1020 to 1022, 1031 to 1033, 1042 to 1043, 1053 to 1055, 1064 to 1066, 1075 to 1077, 1086 to 1088, 1097 to 1099, 1108 to 1110, 1119 to 1121, 1130 to 1132, 1141 to 1143, 1152 to 1154, 1163 to 1165, 1174 to 1176, 1185 to 1187, 1196 to 1198, 1207 to 1209, 1218 to 1220, 1229 to 1231, 1240 to 1242, 1251 to 1253, 1262 to 1264, 1273 to 1275, 1284 to 1286, 1295 to 1297, 1306 to 1308, 1317 to 1319, 1328 to 1330, 1339 to 1341, 1350 to 1352, 1361 to 1363, 1372 to 1374, 1383 to 1385, 1394 to 1396, for BCMA: 1405 to 1407, 1416 to 1418, 1427 to 1429, for CD22: 1492 to 1494, 1503 to 1505, 1514 to 1516, 1525 to 1527, 1536 to 1538, 1547 to 1549, for CD20: 1558 to 1560, 1569 to 1571, 1580 to 1582, 1591 to 1593, 1602 to 1604, 1613 to 1615, 1624 to 1626, 1635 to 1637, 1646 to 1648, and 1657 to 1659.

Also in line with an embodiment of the present invention, the multispecific antigen-binding molecule comprises a first and second binding domain comprising a VH region selected from the group consisting of SEQ ID Nos: for CD123: 39, 50, 61, 72, 83, 94, 105, 116, 127, 138, 149, 160, 171, 182, 193, 204, 215, 226, 237, 248, 259, 270, 281, 292, 303, 314, 325, 336, 347, 358, 369, 380, 391, 402, 413, 424, 435, 446, 457, 468, 479, 490, 501, 512, 523, 534, 545, 556, 567, 578, 589, 600, 611, 622, 633, for FLT3: 644, 655, 666, for CS1: 902, 913, 924, 935, 946, 957, 968, 979, 990, 1001, 1012, 1023, 1034, 1045, 1056, 1067, 1078, 1089, 1100, 1111, 1122, 1133, 1144, 1155, 1166, 1177, 1188, 1199, 1210, 1221, 1232, 1243, 1254, 1265, 1276, 1287, 1298, 1309, 1320, 1331, 1342, 1353, 1364, 1375, 1386, 1397, for BCMA: 1408, 1419, 1430, for CD20: 1495, 1506, 1517, 1528, 1539, for CD20: 1550, 1561, 1572, 1583, 1594, 1605, 1616, 1627, 1638, 1649, and 1660.

Also in line with an embodiment of the present invention, the multispecific antigen-binding molecule comprises a first and/or second binding domain comprising a VL region selected from the group consisting of SEQ ID Nos: for CD123: 40, 51, 62, 73, 84, 95, 106, 117, 128, 139, 150, 161, 172, 183, 194, 205, 216, 227, 238, 249, 260, 271, 282, 293, 304, 315, 326, 337, 348, 359, 370, 381, 392, 403, 414, 425, 436, 447, 458, 469, 480, 491, 502, 513, 524, 535, 546, 557, 568, 579, 590, 601, 612, 623, 634, for FLT3: 645, 656, 667, for CS1: 903, 914, 925, 936, 947, 958, 969, 980, 991, 1002, 1013, 1024, 1035, 1046, 1057, 1068, 1079, 1090, 1101, 1112, 1123, 1134, 1145, 1156, 1167, 1178, 1189, 1200, 1211, 1222, 1233, 1244, 1255, 1266, 1277, 1288, 1299, 1310, 1321, 1332, 1343, 1354, 1365, 1376, 1387, 1398, for BCMA: 1409, 1420, 1431, for CD20: 1496, 1507, 1518, 1529, 1540, 1551, for CD22: 1562, 1573, 1584, 1595, 1606, 1617, 1628, 1639, 1650, and 1661.

In line with this embodiment, the first and second domain which are fused via a peptide linker to a single chain polypeptide comprise a sequence selected from the group consisting of: SEQ ID NO for CD123 41, 52, 63, 74, 85, 96, 107, 118, 129, 140, 151, 162, 173, 184, 195, 206, 217, 228, 239, 250, 261, 272, 283, 294, 305, 316, 327, 338, 349, 360, 371, 382, 393, 404, 415, 426, 437, 448, 459, 470, 481, 492, 503, 514, 525, 536, 547, 558, 569, 580, 591, 602, 613, 624, 635, for FTL3: 646, 657, 668, for FLT3xCD123: 671, 674, 677, 680, 683, 686, 689, 692, 695, 698, 701, 704, 707, 710, 713, 716, 719, 722, 725, 728, 731, 734, 737, 740, 743, 746, 749, 752, 755, 758, 761, 764, 767, 770, 773, 776, 779, 782, 785, 788, 791, 794, 797, 800, 803, 806, 809, 812, 815, 818, 821, 824, 827, 830, 833, for CD123xFLT3: 836, 839, 842, 845, 848, 851, 854, 857, 860, 863, 866, 869, 872, 874, 876, 878, 880, 882, 884, 886, 888, 890, 892, 894, for CS1: 904, 915, 926, 937, 948, 959, 970, 981, 992, 1003, 1014, 1025, 1036, 1047, 1058, 1069, 1080, 1091, 1102, 1113, 1124, 1135, 1146, 1157, 1168, 1179, 1190, 1201, 1212, 1223, 1234, 1245, 1256, 1267, 1278, 1289, 1300, 1311, 1322, 1333, 1344, 1355, 1366, 1377, 1388, 1399, for BCMA: 1410, 1421, 1432, for CS1xBCMA: 1435, for BCMAxCS1: 1438, 1441, 1444, 1447, 1450, 1453, 1456, 1459, 1462, for CS1xBCMA: 1465, 1468, 1471, 1474, 1477, 1480, 1483, 1486, for CD22: 1497, 1508, 1519, 1530, 1541, 1552, for CD20: 1563, 1574, 1585, 1596, 1607, 1618, 1629, 1640, 1651, 1662, for CD22xCD20: 1665, 1668, 1671, 1674, 1677, 1680, 1683, 1686, 1689, 1692, 1695, 1698, 1701, 1704, 1707, 1710, 1713, 1716, 1719, 1722, 1725, 1728, 1731, 1734, 1737, 1740, 1743, 1746, 1749, 1752, for CD20xCD22: 1755, 1758, 1761, 1764, 1767, 1770, 1773, 1776, 1779, 1782, 1785, 1788, 1791, 1794, 1797, 1800, 1803, 1806, 1809, 1812, 1815, 1818, 1821, and 1824, preferably 1399 or 1435 In one aspect the antigen-binding molecule of the invention is characterized by having an amino acid sequence selected from the group consisting of: SEQ ID NO: for CD123: 42, 53, 64, 75, 86, 97, 108, 119, 130, 141, 152, 163, 174, 185, 196, 207, 218, 229, 240, 251, 262, 273, 284, 295, 306, 317, 328, 339, 350, 361, 372, 383, 394, 405, 416, 427, 438, 449, 460, 471, 482, 493, 504, 515, 526, 537, 548, 559, 570, 581, 592, 603, 614, 625, 636, for FLT3: 647, 658, 669, for FLT3xCD123: 672, 675, 678, 681, 684, 687, 690, 693, 696, 699, 702, 705, 708, 711, 714, 717, 720, 723, 726, 729, 732, 735, 738, 741, 744, 747, 750, 753, 756, 759, 762, 765, 768, 771, 774, 777, 780, 783, 786, 789, 792, 795, 798, 801, 804, 807, 810, 813, 816, 819, 822, 825, 828, 831, 834, for CD123xFLT3: 837, 840, 843, 846, 849, 852, 855, 858, 861, 864, 867, 870 873, 875, 877, 879, 881, 883, 885, 887, 889, 891, 893, 895, for CS1: 905, 916, 927, 938, 949, 960, 971, 982, 993, 1004, 1015, 1026, 1037, 1048, 1059, 1070, 1081, 1092, 1103, 1114, 1125, 1136, 1147, 1158, 1169, 1180, 1191, 1202, 1213, 1224, 1235, 1246, 1257, 1268, 1279, 1290, 1301, 1312, 1323, 1334, 1345, 1356, 1367, 1378, 1389, 1400, for BCMA: 1411, 1422, 1433, for CS1xBCMA: 1436, for BCMAxCS1: 1439, 1442, 1445, 1448, 1451, 1454, 1457, 1460, 1463, for CS1xBCMA: 1466, 1469, 1472, 1475, 1478, 1481, 1484, 1487, for CD22: 1498, 1509, 1520, 1531, 1542, 1553, for CD20: 1564, 1575, 1586, 1597, 1608, 1619, 1630, 1641, 1652, 1663, for CD22xCD20: 1666, 1669, 1672, 1675, 1678, 1681, 1684, 1687, 1690, 1693, 1696, 1699, 1702, 1705, 1708, 1711, 1714, 1717, 1720, 1723, 1726, 1729, 1732, 1735, 1738, 1741, 1744, 1747, 1750, 1753, and for CD20xCD22: 1756, 1759, 1762, 1765, 1768, 1771, 1774, 1777, 1780, 1783, 1786, 1789, 1792, 1795, 1798, 1801, 1804, 1807, 1810, 1813, 1816, 1819, 1822, and 1825, preferably 1400 or 1436.

The invention further provides a polynucleotide/nucleic acid molecule encoding an antigen-binding molecule of the invention. A polynucleotide is a biopolymer composed of 13 or more nucleotide monomers covalently bonded in a chain. DNA (such as cDNA) and RNA (such as mRNA) are examples of polynucleotides with distinct biological function. Nucleotides are organic molecules that serve as the monomers or subunits of nucleic acid molecules like DNA or RNA. The nucleic acid molecule or polynucleotide can be double stranded and single stranded, linear and circular. It is preferably comprised in a vector which is preferably comprised in a host cell. Said host cell is, e.g. after transformation or transfection with the vector or the polynucleotide of the invention, capable of expressing the antigen-binding molecule. For that purpose the polynucleotide or nucleic acid molecule is operatively linked with control sequences.

The genetic code is the set of rules by which information encoded within genetic material (nucleic acids) is translated into proteins. Biological decoding in living cells is accomplished by the ribosome which links amino acids in an order specified by mRNA, using tRNA molecules to carry amino acids and to read the mRNA three nucleotides at a time. The code defines how sequences of these nucleotide triplets, called codons, specify which amino acid will be added next during protein synthesis. With some exceptions, a three-nucleotide codon in a nucleic acid sequence specifies a single amino acid. Because the vast majority of genes are encoded with exactly the same code, this particular code is often referred to as the canonical or standard genetic code. While the genetic code determines the protein sequence for a given coding region, other genomic regions can influence when and where these proteins are produced.

Furthermore, the invention provides a vector comprising a polynucleotide/nucleic acid molecule of the invention. A vector is a nucleic acid molecule used as a vehicle to transfer (foreign) genetic material into a cell. The term “vector” encompasses—but is not restricted to—plasmids, viruses, cosmids and artificial chromosomes. In general, engineered vectors comprise an origin of replication, a multicloning site and a selectable marker. The vector itself is generally a nucleotide sequence, commonly a DNA sequence that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding side. Eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding side is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Transfection” is the process of deliberately introducing nucleic acid molecules or polynucleotides (including vectors) into target cells. The term is mostly used for non-viral methods in eukaryotic cells. Transduction is often used to describe virus-mediated transfer of nucleic acid molecules or polynucleotides. Transfection of animal cells typically involves opening transient pores or “holes” in the cell membrane, to allow the uptake of material. Transfection can be carried out using calcium phosphate, by electroporation, by cell squeezing or by mixing a cationic lipid with the material to produce liposomes, which fuse with the cell membrane and deposit their cargo inside.

The term “transformation” is used to describe non-viral transfer of nucleic acid molecules or polynucleotides (including vectors) into bacteria, and also into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be effected by artificial means. For transformation to happen, cells or bacteria must be in a state of competence, which may occur as a time-limited response to environmental conditions such as starvation and cell density.

Moreover, the invention provides a host cell transformed or transfected with the polynucleotide/nucleic acid molecule or with the vector of the invention. As used herein, the terms “host cell” or “recipient cell” are intended to include any individual cell or cell culture that can be or has/have been recipients of vectors, exogenous nucleic acid molecules, and polynucleotides encoding the antigen-binding molecule of the present invention; and/or recipients of the antigen-binding molecule itself. The introduction of the respective material into the cell is carried out by way of transformation, transfection and the like. The term “host cell” is also intended to include progeny or potential progeny of a single cell. Because certain modifications may occur in succeeding generations due to either natural, accidental, or deliberate mutation or due to environmental influences, such progeny may not, in fact, be completely identical (in morphology or in genomic or total DNA complement) to the parent cell, but is still included within the scope of the term as used herein. Suitable host cells include prokaryotic or eukaryotic cells, and also include but are not limited to bacteria, yeast cells, fungi cells, plant cells, and animal cells such as insect cells and mammalian cells, e.g., murine, rat, macaque or human.

The antigen-binding molecule of the invention can be produced in bacteria. After expression, the antigen-binding molecule of the invention is isolated from the E. coli cell paste in a soluble fraction and can be purified through, e.g., affinity chromatography and/or size exclusion. Final purification can be carried out similar to the process for purifying antibody expressed e.g., in CHO cells.

In addition to prokaryotes, eukaryotic microbes such as filamentous fungi or yeast are suitable cloning or expression hosts for the antigen-binding molecule of the invention. Saccharomyces cerevisiae, or common baker's yeast, is the most commonly used among lower eukaryotic host microorganisms. However, a number of other genera, species, and strains are commonly available and useful herein, such as Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of glycosylated antigen-binding molecule of the invention are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodopterafrugiperda cells.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36: 59 (1977)); baby hamster kidney cells (BHK, ATCC CCL 10); Chinese hamster ovary cells/-DHFR (CHO, Urlaub et al., Proc. Natl. Acad. Sci. USA 77: 4216 (1980)); mouse sertoli cells (TM4, Mather, Biol. Reprod. 23: 243-251 (1980)); monkey kidney cells (CVI ATCC CCL 70); African green monkey kidney cells (VERO-76, ATCC CRL1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2,1413 8065); mouse mammary tumor (MMT 060562, ATCC CCL5 1); TRI cells (Mather et al., Annals N. Y Acad. Sci. (1982) 383: 44-68); MRC 5 cells; FS4 cells; and a human hepatoma line (Hep G2).

In a further embodiment the invention provides a process for the production of an antigen-binding molecule of the invention, said process comprising culturing a host cell of the invention under conditions allowing the expression of the antigen-binding molecule of the invention and recovering the produced antigen-binding molecule from the culture.

As used herein, the term “culturing” refers to the in vitro maintenance, differentiation, growth, proliferation and/or propagation of cells under suitable conditions in a medium. The term “expression” includes any step involved in the production of an antigen-binding molecule of the invention including, but not limited to, transcription, post-transcriptional modification, translation, post-translational modification, and secretion.

When using recombinant techniques, the antigen-binding molecule can be produced intracellularly, in the periplasmic space, or directly secreted into the medium. If the antigen-binding molecule is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antigen-binding molecule of the invention prepared from the host cells can be recovered or purified using, for example, hydroxylapatite chromatography, gel electrophoresis, dialysis, and affinity chromatography. Other techniques for protein purification such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromato-focusing, SDS-PAGE, and ammonium sulfate precipitation are also available depending on the antibody to be recovered. Where the antigen-binding molecule of the invention comprises a CH3 domain, the Bakerbond ABX resin (J. T. Baker, Phillipsburg, N.J.) is useful for purification.

Affinity chromatography is a preferred purification technique. The matrix to which the affinity ligand is attached is most often agarose, but other matrices are available. Mechanically stable matrices such as controlled pore glass or poly (styrenedivinyl) benzene allow for faster flow rates and shorter processing times than can be achieved with agarose.

Moreover, the invention provides a pharmaceutical composition comprising an antigen-binding molecule of the invention or an antigen-binding molecule produced according to the process of the invention. It is preferred for the pharmaceutical composition of the invention that the homogeneity of the antigen-binding molecule is ≥80%, more preferably ≥81%, ≥82%, ≥83%, ≥84%, or ≥85%, further preferably ≥86%, ≥87%, ≥88%, ≥89%, or ≥90%, still further preferably, ≥91%, ≥92%, ≥93%, ≥94%, or ≥95% and most preferably ≥96%, ≥97%, ≥98% or ≥99%.

As used herein, the term “pharmaceutical composition” relates to a composition which is suitable for administration to a patient, preferably a human patient. The particularly preferred pharmaceutical composition of this invention comprises one or a plurality of the antigen-binding molecule(s) of the invention, preferably in a therapeutically effective amount. Preferably, the pharmaceutical composition further comprises suitable formulations of one or more (pharmaceutically effective) carriers, stabilizers, excipients, diluents, solubilizers, surfactants, emulsifiers, preservatives and/or adjuvants. Acceptable constituents of the composition are preferably nontoxic to recipients at the dosages and concentrations employed. Pharmaceutical compositions of the invention include, but are not limited to, liquid, frozen, and lyophilized compositions.

The inventive compositions may comprise a pharmaceutically acceptable carrier. In general, as used herein, “pharmaceutically acceptable carrier” means any and all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide pharmaceutical compositions comprising the antigen-binding molecule of the invention and further one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of the invention to improve effectiveness and or to stabilize such formulations and processes against degradation and spoilage due to, for instance, stresses that occur during manufacturing, shipping, storage, pre-use preparation, administration, and thereafter.

In certain embodiments, the pharmaceutical composition may contain formulation materials for the purpose of modifying, maintaining or preserving, e.g., the pH, osmolarity, viscosity, clarity, color, isotonicity, odor, sterility, stability, rate of dissolution or release, adsorption or penetration of the composition (see, REMINGTON'S PHARMACEUTICAL SCIENCES, 18″ Edition, (A. R. Genrmo, ed.), 1990, Mack Publishing Company). In such embodiments, suitable formulation materials may include, but are not limited to:
amino acids such as glycine, alanine, glutamine, asparagine, threonine, proline, 2-phenylalanine, including charged amino acids, preferably lysine, lysine acetate, arginine, glutamate and/or histidine
antimicrobials such as antibacterial and antifungal agents
antioxidants such as ascorbic acid, methionine, sodium sulfite or sodium hydrogen-sulfite;
buffers, buffer systems and buffering agents which are used to maintain the composition at physiological pH or at a slightly lower pH, preferably a lower pH of 4.0 to 6.5; examples of buffers are borate, bicarbonate, Tris-HCl, citrates, phosphates or other organic acids, succinate, phosphate, and histidine; for example Tris buffer of about pH 7.0-8.5;
non-aqueous solvents such as propylene glycol, polyethylene glycol, vegetable oils such as olive oil, and injectable organic esters such as ethyl oleate;
aqueous carriers including water, alcoholic/aqueous solutions, emulsions or suspensions, including saline and buffered media;
biodegradable polymers such as polyesters;
bulking agents such as mannitol or glycine;
chelating agents such as ethylenediamine tetraacetic acid (EDTA);
isotonic and absorption delaying agents;
complexing agents such as caffeine, polyvinylpyrrolidone, beta-cyclodextrin or hydroxypropyl-beta-cyclodextrin)
fillers;
monosaccharides; disaccharides; and other carbohydrates (such as glucose, mannose or dextrins);
carbohydrates may be non-reducing sugars, preferably trehalose, sucrose, octasulfate, sorbitol or xylitol;
(low molecular weight) proteins, polypeptides or proteinaceous carriers such as human or bovine serum albumin, gelatin or immunoglobulins, preferably of human origin;
coloring and flavouring agents;
sulfur containing reducing agents, such as glutathione, thioctic acid, sodium thioglycolate, thioglycerol, [alpha]-monothioglycerol, and sodium thio sulfate
diluting agents;
emulsifying agents;
hydrophilic polymers such as polyvinylpyrrolidone)
salt-forming counter-ions such as sodium;
preservatives such as antimicrobials, anti-oxidants, chelating agents, inert gases and the like;
examples are: benzalkonium chloride, benzoic acid, salicylic acid, thimerosal, phenethyl alcohol, methylparaben, propylparaben, chlorhexidine, sorbic acid or hydrogen peroxide);
metal complexes such as Zn-protein complexes;
solvents and co-solvents (such as glycerin, propylene glycol or polyethylene glycol);
sugars and sugar alcohols, such as trehalose, sucrose, octasulfate, mannitol, sorbitol or xylitol stachyose, mannose, sorbose, xylose, ribose, myoiisitose, galactose, lactitol, ribitol, myoinisitol, galactitol, glycerol, cyclitols (e.g., inositol), polyethylene glycol; and polyhydric sugar alcohols;
suspending agents;
surfactants or wetting agents such as pluronics, PEG, sorbitan esters, polysorbates such as polysorbate 20, polysorbate, triton, tromethamine, lecithin, cholesterol, tyloxapal; surfactants may be detergents, preferably with a molecular weight of >1.2 KD and/or a polyether, preferably with a molecular weight of >3 KD; non-limiting examples for preferred detergents are Tween 20, Tween 40, Tween 60, Tween 80 and Tween 85; non-limiting examples for preferred polyethers are PEG 3000, PEG 3350, PEG 4000 and PEG 5000;
stability enhancing agents such as sucrose or sorbitol;
tonicity enhancing agents such as alkali metal halides, preferably sodium or potassium chloride, mannitol sorbitol;
parenteral delivery vehicles including sodium chloride solution, Ringer's dextrose, dextrose and sodium chloride, lactated Ringer's, or fixed oils;
intravenous delivery vehicles including fluid and nutrient replenishers, electrolyte replenishers (such as those based on Ringer's dextrose).

In the context of the present invention, a pharmaceutical composition, which is preferably a liquid composition or may be a solid composition obtained by lyophilisation or may be a reconstituted liquid composition comprises

(a) an antigen-binding molecule comprising at least three domains, wherein:
a first domain binds to a target cell surface antigen and has an isoelectric point (pI) in the range of 4 to 9,5;
a second domain binds to a second antigen; and has a pI in the range of 8 to 10, preferably 8.5 to 9.0; and
optionally a third domain comprises two polypeptide monomers, each comprising a hinge, a CH2 domain and a CH3 domain, wherein said two polypeptide monomers are fused to each other via a peptide linker;
(b) at least one buffer agent;
(c) at least one saccharide; and
(d) at least one surfactant;
and wherein the pH of the pharmaceutical composition is in the range of 3.5 to 6.

It is further envisaged in the context of the present invention that the at least one buffer agent is present at a concentration range of 5 to 200 mM, more preferably at a concentration range of 10 to 50 mM. It is envisaged in the context of the present invention that the at least one saccharide is selected from the group consisting of monosaccharide, disaccharide, cyclic polysaccharide, sugar alcohol, linear branched dextran or linear non-branched dextran. It is also envisaged in the context of the present invention that the disaccharide is selected from the group consisting of sucrose, trehalose and mannitol, sorbitol, and combinations thereof. It is further envisaged in the context of the present invention that the sugar alcohol is sorbitol. It is envisaged in the context of the present invention that the at least one saccharide is present at a concentration in the range of 1 to 15% (m/V), preferably in a concentration range of 9 to 12% (m/V).

It is also envisaged in the context of the present invention that the at least one surfactant is selected from the group consisting of polysorbate 20, polysorbate 40, polysorbate 60, polysorbate 80, poloxamer 188, pluronic F68, triton X-100, polyoxyethylen, PEG 3350, PEG 4000 and combinations thereof. It is further envisaged in the context of the present invention that the at least one surfactant is present at a concentration in the range of 0.004 to 0.5% (m/V), preferably in the range of 0.001 to 0.01% (m/V). It is envisaged in the context of the present invention that the pH of the composition is in the range of 4.0 to 5.0, preferably 4.2. It is also envisaged in the context of the present invention that the pharmaceutical composition has an osmolarity in the range of 150 to 500 mOsm. It is further envisaged in the context of the present invention that the pharmaceutical composition further comprises an excipient selected from the group consisting of, one or more polyol and one or more amino acid. It is envisaged in the context of the present invention that said one or more excipient is present in the concentration range of 0.1 to 15% (w/V).

It is also envisaged in the context of the present invention that the pharmaceutical composition comprises

(a) the antigen-binding molecule as discussed above,
(b) 10 mM glutamate or acetate,
(c) 9% (m/V) sucrose or 6% (m/V) sucrose and 6% (m/V) hydroxypropyl-β-cyclodextrin,
(d) 0.01% (m/V) polysorbate 80
and wherein the pH of the liquid pharmaceutical composition is 4.2.

It is further envisaged in the context of the present invention that the antigen-binding molecule is present in a concentration range of 0.1 to 8 mg/ml, preferably of 0.2-2.5 mg/ml, more preferably of 0.25-1.0 mg/ml.

It is evident to those skilled in the art that the different constituents of the pharmaceutical composition (e.g., those listed above) can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

It is envisaged that the composition of the invention may comprise, in addition to the polypeptide of the invention defined herein, further biologically active agents, depending on the intended use of the composition. Such agents may be drugs acting on the gastro-intestinal system, drugs acting as cytostatica, drugs preventing hyperurikemia, drugs inhibiting immunoreactions (e.g. corticosteroids), drugs modulating the inflammatory response, drugs acting on the circulatory system and/or agents such as cytokines known in the art. It is also envisaged that the antigen-binding molecule of the present invention is applied in a co-therapy, i.e., in combination with another anti-cancer medicament.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in vivo release and rate of in vivo clearance of the antigen-binding molecule of the invention. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In certain embodiments, the antigen-binding molecule of the invention compositions may be prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the antigen-binding molecule of the invention may be formulated as a lyophilizate using appropriate excipients such as sucrose.

When parenteral administration is contemplated, the therapeutic compositions for use in this invention may be provided in the form of a pyrogen-free, parenterally acceptable aqueous solution comprising the desired antigen-binding molecule of the invention in a pharmaceutically acceptable vehicle. A particularly suitable vehicle for parenteral injection is sterile distilled water in which the antigen-binding molecule of the invention is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired antigen-binding molecule.

Additional pharmaceutical compositions will be evident to those skilled in the art, including formulations involving the antigen-binding molecule of the invention in sustained- or controlled-delivery/release formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP 058481), copolymers of L-glutamic acid and gamma ethyl-L-glutamate (Sidman et al., 1983, Biopolymers 2:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP 133,988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP 036,676; EP 088,046 and EP 143,949.

The antigen-binding molecule may also be entrapped in microcapsules prepared, for example, by coacervation techniques or by interfacial polymerization (for example, hydroxymethylcellulose or gelatine-microcapsules and poly (methylmethacylate) microcapsules, respectively), in colloidal drug delivery systems (for example, liposomes, albumin microspheres, microemulsions, nanoparticles and nanocapsules), or in macroemulsions. Such techniques are disclosed in Remington's Pharmaceutical Sciences, 16th edition, Oslo, A., Ed., (1980).

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Another aspect of the invention includes self-buffering antigen-binding molecule of the invention formulations, which can be used as pharmaceutical compositions, as described in international patent application WO 06138181A2 (PCT/US2006/022599). A variety of expositions are available on protein stabilization and formulation materials and methods useful in this regard, such as Arakawa et al., “Solvent interactions in pharmaceutical formulations,” Pharm Res. 8(3): 285-91 (1991); Kendrick et al., “Physical stabilization of proteins in aqueous solution” in: RATIONAL DESIGN OF STABLE PROTEIN FORMULATIONS: THEORY AND PRACTICE, Carpenter and Manning, eds. Pharmaceutical Biotechnology. 13: 61-84 (2002), and Randolph et al., “Surfactant-protein interactions”, Pharm Biotechnol. 13: 159-75 (2002), see particularly the parts pertinent to excipients and processes of the same for self-buffering protein formulations in accordance with the current invention, especially as to protein pharmaceutical products and processes for veterinary and/or human medical uses.

Salts may be used in accordance with certain embodiments of the invention to, for example, adjust the ionic strength and/or the isotonicity of a formulation and/or to improve the solubility and/or physical stability of a protein or other ingredient of a composition in accordance with the invention. As is well known, ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, in particular, the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. A number of categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic”. Destabilizing solutes are referred to as “chaotropic”. Kosmotropes commonly are used at high concentrations (e.g., >1 molar ammonium sulfate) to precipitate proteins from solution (“salting-out”). Chaotropes commonly are used to denture and/or to solubilize proteins (“salting-in”). The relative effectiveness of ions to “salt-in” and “salt-out” defines their position in the Hofmieister series.

Free amino acids can be used in the antigen-binding molecule of the invention formulations in accordance with various embodiments of the invention as bulking agents, stabilizers, and antioxidants, as well as other standard uses. Lysine, proline, serine, and alanine can be used for stabilizing proteins in a formulation. Glycine is useful in lyophilization to ensure correct cake structure and properties. Arginine may be useful to inhibit protein aggregation, in both liquid and lyophilized formulations. Methionine is useful as an antioxidant.

Polyols include sugars, e.g., mannitol, sucrose, and sorbitol and polyhydric alcohols such as, for instance, glycerol and propylene glycol, and, for purposes of discussion herein, polyethylene glycol (PEG) and related substances. Polyols are kosmotropic. They are useful stabilizing agents in both liquid and lyophilized formulations to protect proteins from physical and chemical degradation processes. Polyols also are useful for adjusting the tonicity of formulations. Among polyols useful in select embodiments of the invention is mannitol, commonly used to ensure structural stability of the cake in lyophilized formulations. It ensures structural stability to the cake. It is generally used with a lyoprotectant, e.g., sucrose. Sorbitol and sucrose are among preferred agents for adjusting tonicity and as stabilizers to protect against freeze-thaw stresses during transport or the preparation of bulks during the manufacturing process. Reducing sugars (which contain free aldehyde or ketone groups), such as glucose and lactose, can glycate surface lysine and arginine residues. Therefore, they generally are not among preferred polyols for use in accordance with the invention. In addition, sugars that form such reactive species, such as sucrose, which is hydrolyzed to fructose and glucose under acidic conditions, and consequently engenders glycation, also is not among preferred polyols of the invention in this regard. PEG is useful to stabilize proteins and as a cryoprotectant and can be used in the invention in this regard.

Embodiments of the antigen-binding molecule of the invention formulations further comprise surfactants. Protein molecules may be susceptible to adsorption on surfaces and to denaturation and consequent aggregation at air-liquid, solid-liquid, and liquid-liquid interfaces. These effects generally scale inversely with protein concentration. These deleterious interactions generally scale inversely with protein concentration and typically are exacerbated by physical agitation, such as that generated during the shipping and handling of a product. Surfactants routinely are used to prevent, minimize, or reduce surface adsorption. Useful surfactants in the invention in this regard include polysorbate 20, polysorbate 80, other fatty acid esters of sorbitan polyethoxylates, and poloxamer 188. Surfactants also are commonly used to control protein conformational stability. The use of surfactants in this regard is protein-specific since, any given surfactant typically will stabilize some proteins and destabilize others.

Polysorbates are susceptible to oxidative degradation and often, as supplied, contain sufficient quantities of peroxides to cause oxidation of protein residue side-chains, especially methionine. Consequently, polysorbates should be used carefully, and when used, should be employed at their lowest effective concentration. In this regard, polysorbates exemplify the general rule that excipients should be used in their lowest effective concentrations.

Embodiments of the antigen-binding molecule of the invention formulations further comprise one or more antioxidants. To some extent deleterious oxidation of proteins can be prevented in pharmaceutical formulations by maintaining proper levels of ambient oxygen and temperature and by avoiding exposure to light. Antioxidant excipients can be used as well to prevent oxidative degradation of proteins. Among useful antioxidants in this regard are reducing agents, oxygen/free-radical scavengers, and chelating agents. Antioxidants for use in therapeutic protein formulations in accordance with the invention preferably are water-soluble and maintain their activity throughout the shelf life of a product. EDTA is a preferred antioxidant in accordance with the invention in this regard. Antioxidants can damage proteins. For instance, reducing agents, such as glutathione in particular, can disrupt intramolecular disulfide linkages. Thus, antioxidants for use in the invention are selected to, among other things, eliminate or sufficiently reduce the possibility of themselves damaging proteins in the formulation.

Formulations in accordance with the invention may include metal ions that are protein co-factors and that are necessary to form protein coordination complexes, such as zinc necessary to form certain insulin suspensions. Metal ions also can inhibit some processes that degrade proteins. However, metal ions also catalyze physical and chemical processes that degrade proteins. Magnesium ions (10-120 mM) can be used to inhibit isomerization of aspartic acid to isoaspartic acid. Ca⁺² ions (up to 100 mM) can increase the stability of human deoxyribonuclease. Mg⁺², Mn⁺², and Zn+2 however, can destabilize rhDNase. Similarly, Ca⁺² and Sr⁺² can stabilize Factor VIII, it can be destabilized by Mg⁺², Mn⁺² and Zn⁺², Cu⁺² and Fe⁺², and its aggregation can be increased by Al⁺³ ions.

Embodiments of the antigen-binding molecule of the invention formulations further comprise one or more preservatives. Preservatives are necessary when developing multi-dose parenteral formulations that involve more than one extraction from the same container. Their primary function is to inhibit microbial growth and ensure product sterility throughout the shelf-life or term of use of the drug product. Commonly used preservatives include benzyl alcohol, phenol and m-cresol. Although preservatives have a long history of use with small-molecule parenterals, the development of protein formulations that includes preservatives can be challenging. Preservatives almost always have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. At least four such pen devices containing preserved formulations of hGH are currently available on the market. Norditropin (liquid, Novo Nordisk), Nutropin AQ (liquid, Genentech) & Genotropin (lyophilized—dual chamber cartridge, Pharmacia & Upjohn) contain phenol while Somatrope (Eli Lilly) is formulated with m-cresol. Several aspects need to be considered during the formulation and development of preserved dosage forms. The effective preservative concentration in the drug product must be optimized. This requires testing a given preservative in the dosage form with concentration ranges that confer anti-microbial effectiveness without compromising protein stability.

As may be expected, development of liquid formulations containing preservatives are more challenging than lyophilized formulations. Freeze-dried products can be lyophilized without the preservative and reconstituted with a preservative containing diluent at the time of use. This shortens the time for which a preservative is in contact with the protein, significantly minimizing the associated stability risks. With liquid formulations, preservative effectiveness and stability should be maintained over the entire product shelf-life (about 18 to 24 months). An important point to note is that preservative effectiveness should be demonstrated in the final formulation containing the active drug and all excipient components.

The antigen-binding molecules disclosed herein may also be formulated as immuno-liposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antigen-binding molecule are prepared by methods known in the art, such as described in Epstein et al., Proc. Natl. Acad. Sci. USA, 82: 3688 (1985); Hwang et al., Proc. Natl Acad. Sci. USA, 77: 4030 (1980); U.S. Pat. Nos. 4,485,045 and 4,544,545; and WO 97/38731. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antigen-binding molecule of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst. 81 (19) 1484 (1989).

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration.

The biological activity of the pharmaceutical composition defined herein can be determined for instance by cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the pharmaceutical composition of the invention, using e.g. standardized NCI response criteria. The success or in vivo efficacy of the therapy using a pharmaceutical composition of the invention refers to the effectiveness of the composition for its intended purpose, i.e. the ability of the composition to cause its desired effect, i.e. depletion of pathologic cells, e.g. tumor cells. The in vivo efficacy may be monitored by established standard methods for the respective disease entities including, but not limited to white blood cell counts, differentials, Fluorescence Activated Cell Sorting, bone marrow aspiration. In addition, various disease specific clinical chemistry parameters and other established standard methods may be used. Furthermore, computer-aided tomography, X-ray, nuclear magnetic resonance tomography (e.g. for National Cancer Institute-criteria based response assessment [Cheson B D, Homing S J, Coiffier B, Shipp M A, Fisher R I, Connors J M, Lister T A, Vose J, Grillo-Lopez A, Hagenbeek A, Cabanillas F, Klippensten D, Hiddemann W, Castellino R, Harris N L, Armitage J O, Carter W, Hoppe R, Canellos GP. Report of an international workshop to standardize response criteria for non-Hodgkin's lymphomas. NCI Sponsored International Working Group. J Clin Oncol. 1999 April; 17(4):1244]), positron-emission tomography scanning, white blood cell counts, differentials, Fluorescence Activated Cell Sorting, bone marrow aspiration, lymph node biopsies/histologies, and various lymphoma specific clinical chemistry parameters (e.g. lactate dehydrogenase) and other established standard methods may be used.

Another major challenge in the development of drugs such as the pharmaceutical composition of the invention is the predictable modulation of pharmacokinetic properties. To this end, a pharmacokinetic profile of the drug candidate, i.e. a profile of the pharmacokinetic parameters that affect the ability of a particular drug to treat a given condition, can be established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above. It is an envisaged characteristic of the antigen-binding molecules of the present invention provided with the specific FC modality that they comprise, for example, differences in pharmacokinetic behavior. A half-life extended targeting antigen-binding molecule according to the present invention preferably shows a surprisingly increased residence time in vivo in comparison to “canonical” non-HLE versions of said antigen-binding molecule.

“Half-life” means the time where 50% of an administered drug are eliminated through biological processes, e.g. metabolism, excretion, etc. By “hepatic first-pass metabolism” is meant the propensity of a drug to be metabolized upon first contact with the liver, i.e. during its first pass through the liver. “Volume of distribution” means the degree of retention of a drug throughout the various compartments of the body, like e.g. intracellular and extracellular spaces, tissues and organs, etc. and the distribution of the drug within these compartments. “Degree of blood serum binding” means the propensity of a drug to interact with and bind to blood serum proteins, such as albumin, leading to a reduction or loss of biological activity of the drug.

Pharmacokinetic parameters also include bioavailability, lag time (Tlag), Tmax, absorption rates, more onset and/or Cmax for a given amount of drug administered. “Bioavailability” means the amount of a drug in the blood compartment. “Lag time” means the time delay between the administration of the drug and its detection and measurability in blood or plasma. “Tmax” is the time after which maximal blood concentration of the drug is reached, and “Cmax” is the blood concentration maximally obtained with a given drug. The time to reach a blood or tissue concentration of the drug which is required for its biological effect is influenced by all parameters. Pharmacokinetic parameters of bispecific antigen-binding molecules exhibiting cross-species specificity, which may be determined in preclinical animal testing in non-chimpanzee primates as outlined above, are also set forth e.g. in the publication by Schlereth et al. (Cancer Immunol. Immunother. 20 (2005), 1-12).

In a preferred aspect of the invention the pharmaceutical composition is stable for at least four weeks at about −20° C. As apparent from the appended examples the quality of an antigen-binding molecule of the invention vs. the quality of corresponding state of the art antigen-binding molecules may be tested using different systems. Those tests are understood to be in line with the “ICH Harmonised Tripartite Guideline: Stability Testing of Biotechnological Biological Products Q5C and Specifications: Test procedures and Acceptance Criteria for Biotech Biotechnological Biological Products Q6B” and, thus are elected to provide a stability-indicating profile that provides certainty that changes in the identity, purity and potency of the product are detected. It is well accepted that the term purity is a relative term. Due to the effect of glycosylation, deamidation, or other heterogeneities, the absolute purity of a biotechnological/biological product should be typically assessed by more than one method and the purity value derived is method-dependent. For the purpose of stability testing, tests for purity should focus on methods for determination of degradation products.

For the assessment of the quality of a pharmaceutical composition comprising an antigen-binding molecule of the invention may be analyzed e.g. by analyzing the content of soluble aggregates in a solution (HMWS per size exclusion). It is preferred that stability for at least four weeks at about −20° C. is characterized by a content of less than 1.5% HMWS, preferably by less than 1% HMWS.

A preferred formulation for the antigen-binding molecule as a pharmaceutical composition may e.g. comprise the components of a formulation as described below:
Formulation:
   potassium phosphate, L-arginine hydrochloride, trehalose dihydrate, polysorbate 80 at pH 6.0

Other examples for the assessment of the stability of an antigen-binding molecule of the invention in form of a pharmaceutical composition are provided in the appended examples 4-12. In those examples embodiments of antigen-binding molecules of the invention are tested with respect to different stress conditions in different pharmaceutical formulations and the results compared with other half-life extending (HLE) formats of bispecific T cell engaging antigen-binding molecule known from the art. In general, it is envisaged that antigen-binding molecules provided with the specific FC modality according to the present invention are typically more stable over a broad range of stress conditions such as temperature and light stress, both compared to antigen-binding molecules provided with different HLE formats and without any HLE format (e.g. “canonical” antigen-binding molecules). Said temperature stability may relate both to decreased (below room temperature including freezing) and increased (above room temperature including temperatures up to or above body temperature) temperature. As the person skilled in the art will acknowledge, such improved stability with regard to stress, which is hardly avoidable in clinical practice, makes the antigen-binding molecule safer because less degradation products will occur in clinical practice. In consequence, said increased stability means increased safety.

One embodiment provides the antigen-binding molecule of the invention or the antigen-binding molecule produced according to the process of the invention for use in the prevention, treatment or amelioration of a cancer correlating with CS1, BCMA, CD20, CD22, FLT3, CD123, CLL1, MSLN, or EpCAM expression or CS1, BCMA, CD20, CD22, FLT3, CD123, CLL1, MSLN, or EpCAM overexpression, such as prostate cancer.

The formulations described herein are useful as pharmaceutical compositions in the treatment, amelioration and/or prevention of the pathological medical condition as described herein in a patient in need thereof. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “amelioration” as used herein refers to any improvement of the disease state of a patient having a disease as specified herein below, by the administration of an antigen-binding molecule according to the invention to a subject in need thereof. Such an improvement may also be seen as a slowing or stopping of the progression of the patient's disease. The term “prevention” as used herein means the avoidance of the occurrence or re-occurrence of a patient having a tumor or cancer or a metastatic cancer as specified herein below, by the administration of an antigen-binding molecule according to the invention to a subject in need thereof.

The term “disease” refers to any condition that would benefit from treatment with the antigen-binding molecule or the pharmaceutic composition described herein. This includes chronic and acute disorders or diseases including those pathological conditions that predispose the mammal to the disease in question.

A “neoplasm” is an abnormal growth of tissue, usually but not always forming a mass. When also forming a mass, it is commonly referred to as a “tumor”. Neoplasms or tumors or can be benign, potentially malignant (pre-cancerous), or malignant. Malignant neoplasms are commonly called cancer. They usually invade and destroy the surrounding tissue and may form metastases, i.e., they spread to other parts, tissues or organs of the body. Hence, the term “metatstatic cancer” encompasses metastases to other tissues or organs than the one of the original tumor. Lymphomas and leukemias are lymphoid neoplasms. For the purposes of the present invention, they are also encompassed by the terms “tumor” or “cancer”.

The term “viral disease” describes diseases, which are the result of a viral infection of a subject.

The term “immunological disorder” as used herein describes in line with the common definition of this term immunological disorders such as autoimmune diseases, hypersensitivities, immune deficiencies.

In one embodiment the invention provides a method for the treatment or amelioration of a cancer correlating with CS1, BCMA, CD20, CD22, FLT3, CD123, CLL1, MSLN, or EpCAM expression or CS1, BCMA, CD20, CD22, FLT3, CD123, CLL1, MSLN, or EpCAM overexpression, comprising the step of administering to a subject in need thereof the antigen-binding molecule of the invention, or the antigen-binding molecule produced according to the process of the invention. The CS1, BCMA, CD20, CD22, FLT3, CD123, CLL1, MSLN, or EpCAMxCD3 bispecific single chain antibody is particularly advantageous for the therapy of cancer, preferably solid tumors, more preferably carcinomas and prostate cancer.

The terms “subject in need” or those “in need of treatment” includes those already with the disorder, as well as those in which the disorder is to be prevented. The subject in need or “patient” includes human and other mammalian subjects that receive either prophylactic or therapeutic treatment.

The antigen-binding molecule of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration.

Formulations and compositions thus may be designed in accordance with the invention for delivery by any suitable route of administration. In the context of the present invention, the routes of administration include, but are not limited to
topical routes (such as epicutaneous, inhalational, nasal, opthalmic, auricular/aural, vaginal, mucosal);
enteral routes (such as oral, gastrointestinal, sublingual, sublabial, buccal, rectal); and
parenteral routes (such as intravenous, intraarterial, intraosseous, intramuscular, intracerebral, intracerebroventricular, epidural, intrathecal, subcutaneous, intraperitoneal, extra-amniotic, intraarticular, intracardiac, intradermal, intralesional, intrauterine, intravesical, intravitreal, transdermal, intranasal, transmucosal, intrasynovial, intraluminal).

The pharmaceutical compositions and the antigen-binding molecule of this invention are particularly useful for parenteral administration, e.g., subcutaneous or intravenous delivery, for example by injection such as bolus injection, or by infusion such as continuous infusion. Pharmaceutical compositions may be administered using a medical device. Examples of medical devices for administering pharmaceutical compositions are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163.

In particular, the present invention provides for an uninterrupted administration of the suitable composition. As a non-limiting example, uninterrupted or substantially uninterrupted, i.e. continuous administration may be realized by a small pump system worn by the patient for metering the influx of therapeutic agent into the body of the patient. The pharmaceutical composition comprising the antigen-binding molecule of the invention can be administered by using said pump systems. Such pump systems are generally known in the art, and commonly rely on periodic exchange of cartridges containing the therapeutic agent to be infused. When exchanging the cartridge in such a pump system, a temporary interruption of the otherwise uninterrupted flow of therapeutic agent into the body of the patient may ensue. In such a case, the phase of administration prior to cartridge replacement and the phase of administration following cartridge replacement would still be considered within the meaning of the pharmaceutical means and methods of the invention together make up one “uninterrupted administration” of such therapeutic agent.

The continuous or uninterrupted administration of the antigen-binding molecules of the invention may be intravenous or subcutaneous by way of a fluid delivery device or small pump system including a fluid driving mechanism for driving fluid out of a reservoir and an actuating mechanism for actuating the driving mechanism. Pump systems for subcutaneous administration may include a needle or a cannula for penetrating the skin of a patient and delivering the suitable composition into the patient's body. Said pump systems may be directly fixed or attached to the skin of the patient independently of a vein, artery or blood vessel, thereby allowing a direct contact between the pump system and the skin of the patient. The pump system can be attached to the skin of the patient for 24 hours up to several days. The pump system may be of small size with a reservoir for small volumes. As a non-limiting example, the volume of the reservoir for the suitable pharmaceutical composition to be administered can be between 0.1 and 50 ml.

The continuous administration may also be transdermal by way of a patch worn on the skin and replaced at intervals. One of skill in the art is aware of patch systems for drug delivery suitable for this purpose. It is of note that transdermal administration is especially amenable to uninterrupted administration, as exchange of a first exhausted patch can advantageously be accomplished simultaneously with the placement of a new, second patch, for example on the surface of the skin immediately adjacent to the first exhausted patch and immediately prior to removal of the first exhausted patch. Issues of flow interruption or power cell failure do not arise.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

The compositions of the present invention can be administered to the subject at a suitable dose which can be determined e.g. by dose escalating studies by administration of increasing doses of the antigen-binding molecule of the invention exhibiting cross-species specificity described herein to non-chimpanzee primates, for instance macaques. As set forth above, the antigen-binding molecule of the invention exhibiting cross-species specificity described herein can be advantageously used in identical form in preclinical testing in non-chimpanzee primates and as drug in humans. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts or doses effective for this use will depend on the condition to be treated (the indication), the delivered antigen-binding molecule, the therapeutic context and objectives, the severity of the disease, prior therapy, the patient's clinical history and response to the therapeutic agent, the route of administration, the size (body weight, body surface or organ size) and/or condition (the age and general health) of the patient, and the general state of the patient's own immune system. The proper dose can be adjusted according to the judgment of the attending physician such that it can be administered to the patient once or over a series of administrations, and in order to obtain the optimal therapeutic effect.

A typical dosage may range from about 0.1 μg/kg to up to about 30 mg/kg or more, depending on the factors mentioned above. In specific embodiments, the dosage may range from 1.0 μg/kg up to about 20 mg/kg, optionally from 10 μg/kg up to about 10 mg/kg or from 100 μg/kg up to about 5 mg/kg.

A therapeutic effective amount of an antigen-binding molecule of the invention preferably results in a decrease in severity of disease symptoms, an increase in frequency or duration of disease symptom-free periods or a prevention of impairment or disability due to the disease affliction. For treating diseases correlating with CS1, BCMA, CD20, CD22, FLT3, CD123, CLL1, MSLN, or EpCAM expression as described herein above, a therapeutically effective amount of the antigen-binding molecule of the invention, here: an anti-CS1, BCMA, CD20, CD22, FLT3, CD123, CLL1, MSLN, or EpCAM/anti-CD3 antigen-binding molecule, preferably inhibits cell growth or tumor growth by at least about 20%, at least about 40%, at least about 50%, at least about 60%, at least about 70%, at least about 80%, or at least about 90% relative to untreated patients. The ability of a compound to inhibit tumor growth may be evaluated in an animal model predictive of efficacy

The pharmaceutical composition can be administered as a sole therapeutic or in combination with additional therapies such as anti-cancer therapies as needed, e.g. other proteinaceous and non-proteinaceous drugs. These drugs may be administered simultaneously with the composition comprising the antigen-binding molecule of the invention as defined herein or separately before or after administration of said antigen-binding molecule in timely defined intervals and doses.

The term “effective and non-toxic dose” as used herein refers to a tolerable dose of an inventive antigen-binding molecule which is high enough to cause depletion of pathologic cells, tumor elimination, tumor shrinkage or stabilization of disease without or essentially without major toxic effects. Such effective and non-toxic doses may be determined e.g. by dose escalation studies described in the art and should be below the dose inducing severe adverse side events (dose limiting toxicity, DLT).

The term “toxicity” as used herein refers to the toxic effects of a drug manifested in adverse events or severe adverse events. These side events may refer to a lack of tolerability of the drug in general and/or a lack of local tolerance after administration. Toxicity could also include teratogenic or carcinogenic effects caused by the drug.

The term “safety”, “in vivo safety” or “tolerability” as used herein defines the administration of a drug without inducing severe adverse events directly after administration (local tolerance) and during a longer period of application of the drug. “Safety”, “in vivo safety” or “tolerability” can be evaluated e.g. at regular intervals during the treatment and follow-up period. Measurements include clinical evaluation, e.g. organ manifestations, and screening of laboratory abnormalities. Clinical evaluation may be carried out and deviations to normal findings recorded/coded according to NCI-CTC and/or MedDRA standards. Organ manifestations may include criteria such as allergy/immunology, blood/bone marrow, cardiac arrhythmia, coagulation and the like, as set forth e.g. in the Common Terminology Criteria for adverse events v3.0 (CTCAE). Laboratory parameters which may be tested include for instance hematology, clinical chemistry, coagulation profile and urine analysis and examination of other body fluids such as serum, plasma, lymphoid or spinal fluid, liquor and the like. Safety can thus be assessed e.g. by physical examination, imaging techniques (i.e. ultrasound, x-ray, CT scans, Magnetic Resonance Imaging (MRI), other measures with technical devices (i.e. electrocardiogram), vital signs, by measuring laboratory parameters and recording adverse events. For example, adverse events in non-chimpanzee primates in the uses and methods according to the invention may be examined by histopathological and/or histochemical methods.

The above terms are also referred to e.g. in the Preclinical safety evaluation of biotechnology-derived pharmaceuticals S6; ICH Harmonised Tripartite Guideline; ICH Steering Committee meeting on Jul. 16, 1997.

Finally, the invention provides a kit comprising an antigen-binding molecule of the invention or produced according to the process of the invention, a pharmaceutical composition of the invention, a polynucleotide of the invention, a vector of the invention and/or a host cell of the invention.

In the context of the present invention, the term “kit” means two or more components—one of which corresponding to the antigen-binding molecule, the pharmaceutical composition, the vector or the host cell of the invention—packaged together in a container, recipient or otherwise. A kit can hence be described as a set of products and/or utensils that are sufficient to achieve a certain goal, which can be marketed as a single unit.

The kit may comprise one or more recipients (such as vials, ampoules, containers, syringes, bottles, bags) of any appropriate shape, size and material (preferably waterproof, e.g. plastic or glass) containing the antigen-binding molecule or the pharmaceutical composition of the present invention in an appropriate dosage for administration (see above). The kit may additionally contain directions for use (e.g. in the form of a leaflet or instruction manual), means for administering the antigen-binding molecule of the present invention such as a syringe, pump, infuser or the like, means for reconstituting the antigen-binding molecule of the invention and/or means for diluting the antigen-binding molecule of the invention.

The invention also provides kits for a single-dose administration unit. The kit of the invention may also contain a first recipient comprising a dried/lyophilized antigen-binding molecule and a second recipient comprising an aqueous formulation. In certain embodiments of this invention, kits containing single-chambered and multi-chambered pre-filled syringes (e.g., liquid syringes and lyosyringes) are provided.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended to limit the scope of the present invention in any way.

### EXAMPLES

### Example 1: Modeling of Inter-Domain Space Dependent on Linker Length

A dual targeting MSLNxEpCAM HLE BiTE® molecule has been modelled for interdomain space at various linker lengths based on structural data of the complexed MSLN and of the extracellular portion of EpCAM as an example, which can be generalized to any multitargeting antigen-binding molecule according to the present invention. The linkers were modeled using the software tool “Coot” (version 0.3.3 MRC Laboratory of Molecular Biology, Cambridge, UK), starting from a model scFv (i.e. the crystal structure of I2C) and electron density used as guide in areas adjacent to the surface of the protein. Areas further than 5 Å away were modeled using the typical backbone peptide bond distance and regularized every 5 residue additions to maintain geometry. Linker length of several residues were thus constructed and patched onto the multitargeting HLE BiTE® model. The software suite Schrodinger (version 2019-2, Schrödinger, NY, US) was used to energy minimize models after introduction of the linker and to position the molecules in different regions while maintaining linker length and geometry to best mimic the most likely situation in reality. The linkers were allowed to extend to maximum, without compromising geometry, Hence, the modeling data is indicative for a minimum linker length that could, as a minimum, avoid steric hindrance upon full linker extension, wherein linker compares to a spring which can extend or wrap itself). Accordingly, a linker below a certain threshold, would not be able to avoid steric hindrance no matter how much it extends itself.

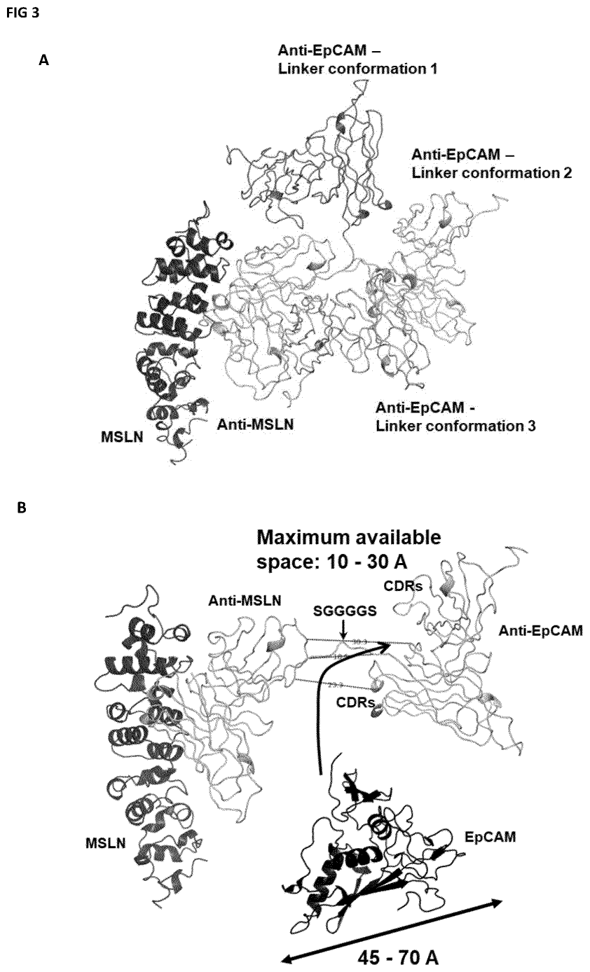

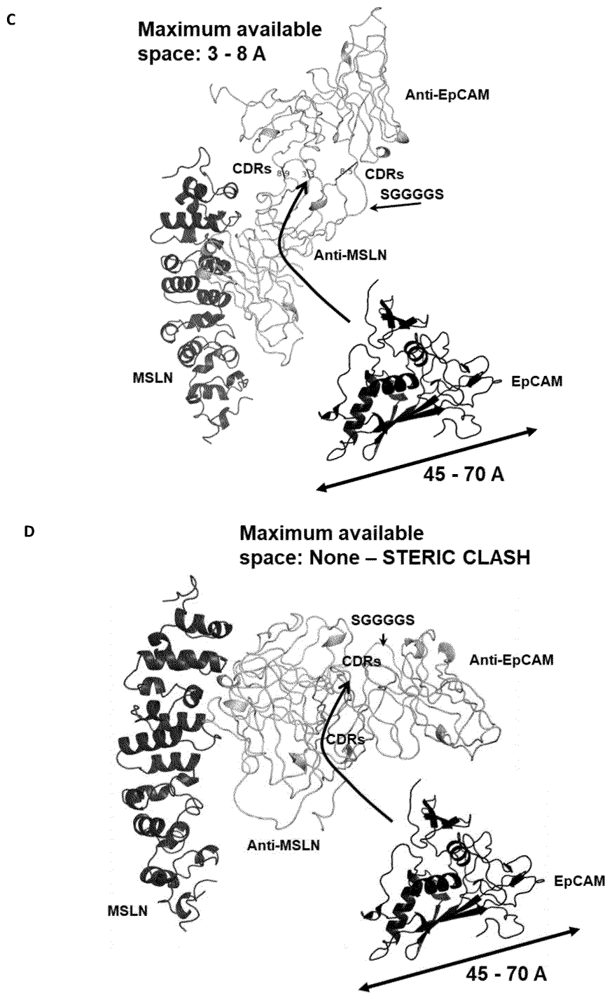

In a first model, a SGGGGS linker was modeled between the two target binding domains which are scFvs and a (GGGGS)3 linker between the VH and VL within the binding domains, respectively. Each linker was regularized and minimized. The SGGGGS linker was based on the theoretical and known ca-ca distance in the peptidic bond, corroborated by structure. The linker was conformationally probed (using MD) around a sphere with a radius of 19 Å, which corresponds to the extended linker (maximum length). Keeping the first binding domain, i.e. the anti-MSLN binding domain in the present example, fixed, three conformations are shown (FIGS. 3A, B and C) where the linker swings in different orthogonal directions and the resulting anti EpCAM scFv position is shown (linker conformation 1, 2 and 3, respectively). In case of linker position 3, a complete clash is observed, while in positions 1 and 2, no clash but is observed. However, the space is still not enough to accommodate the TAA2 EpCAM based on where the CDRs are located in the second binding domain of the multitargeting antigen-binding molecule. Hence, this result strongly indicates the need of a longer linker between the two target binding domains. If the skilled person used the size of target EpCAM as guide, one would predict a better linker to be one that has at least about 20 residues (i.e. 70 A divided by 3.8).

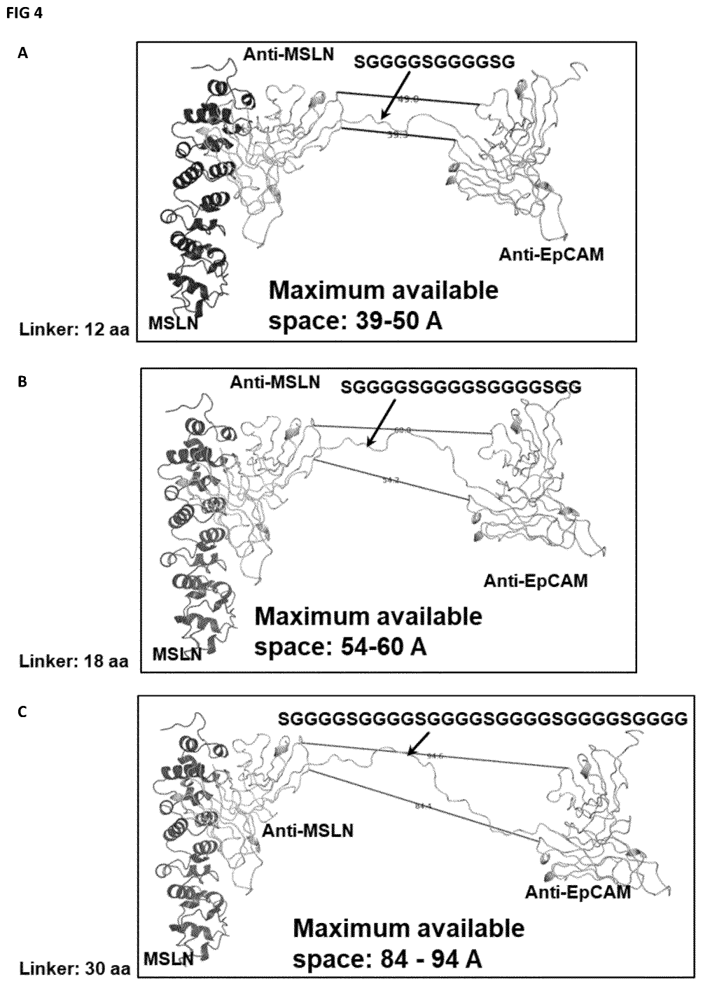

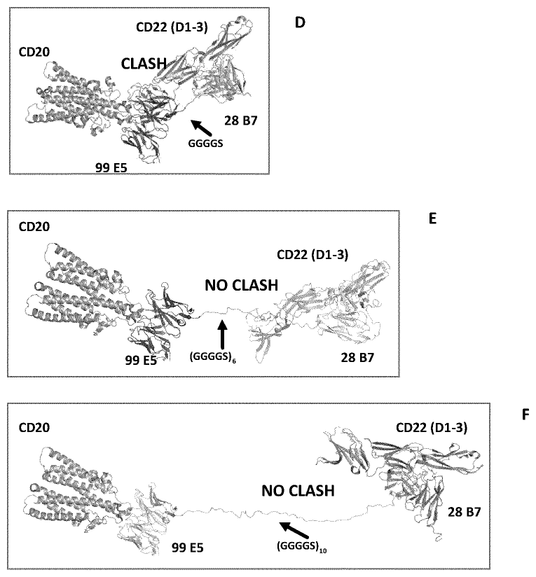

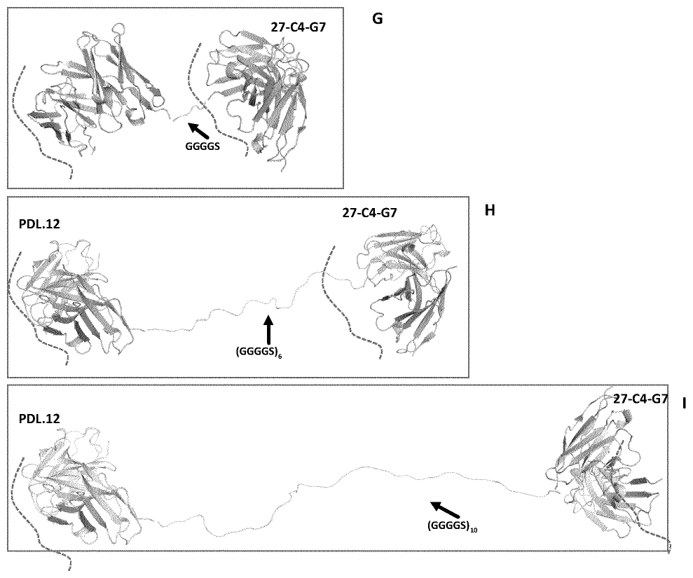

In order to better quantify what linker lengths would be expected to work and not to work in the context of a multitargeting antigen-binding molecule in the context of the present invention, as exemplified by a dual targeting MSLNxEpCAM HLE BiTE®, the linker length between the first and the second target binder was varied at 12, 18 and 30 amino acids and the resulting space between the two target binding domains was modeled as explained above with respect to conformation 2. In conclusion, lack of space renders a short linker solution such as a SGGGS linker between the two target binders according to the present invention a non-obvious choice for this setup of target binders in a multitargeting antigen-binding molecule, in particular a dual targeting BiTE® molecule. FIG. 4A shows that a linker of 12 aa (SGGGGSGGGGSG) offers a maximum available space of 39 to 50 Å which would not accommodate the target to be bound at 45 to 70 Å. Also, an 18 aa long linker (SGGGGSGGGGSGGGGSGG) with a maximum available space between binding domains in a setup as described herein of 54 to 60 Awould likely not allow binding to the second TAA2 of an exemplary size of 45 to 70 Å (FIG. 4B). In contrast, a 30 aa long linker would offer 84 to 94 Å of maximum space, thus safely allowing the target binder to bind its exemplified target EpCAM of about 45 to 70 Å. Thus, the skilled person would have chosen a linker length at least greater than 18 aa to ensure binding of the second TAA2 by the dual targeting MSLNxEpCAM HLE BiTE® molecule as an example for a multispecific antigen-binding molecule according to the present invention. FIGS. 4D to F show a comparable modeling for a dual targeting CD20xCD22 HLE BiTE (SEQ ID NO: 1817). CD20 epitope conformation is derived from PDB ID 6Y9A, and CD22 epitope conformation is derived from PDB ID 5VL3. The linker between the two target binders is GGGGS (Linker distance span: 3-8 Å, available CDR space for CD22 binding 35-40 Å) which is expected lead to a steric clash (D). For a linker (GGGGS)6, the linker distance span is larger, with available CDR space for the CD22 binding of 35-80 A (E). For a linker (GGGGS)10, the linker distance span is larger, with available CDR space for the CD22 binding of 35˜150 A (F). The situation in (E) and (F) is not supposed to lead to a steric clash and based on the modelling, would be preferred over (D), which is unexpectedly not the case as demonstrated in practical experiments. FIGS. 4G to I show a comparable modeling for a dual targeting CS1xCD22 HLE BiTE (SEQ ID NO: 1437). The linker between the two target binders is GGGGS (Linker distance span: 3-8 Å, available CDR space for BCMA binding 35˜40 A) which is prone to lead to a steric clash (G). For a linker (GGGGS)6, the linker distance span is larger, with available CDR space for the BCMA binding of 35-80 A (H). For a linker (GGGGS)10, the linker distance span is larger, with available CDR space for the BCMA binding of 35˜150 A (I). The situation in (H) and (I) is not supposed to lead to a steric clash and based on the modelling, would be preferred over (G), which is unexpectedly not the case as demonstrated in practical experiments.

### Example 2: Determination of Simultaneous Binding of Target Binding Domains of Multitargeting Antigen-Binding Molecules

Simultaneous dual binding was determined by electrically switchable nanolever measurements which were performed on a DRX2 instrument, using a multi-purpose 96 bp dual color biochip (switchSENSE® by Dynamic Biosensors GmbH; Planegg, Germany). Prior to kinetic experiments, target antigens EpCAM and MSLN were conjugated to 96 bp DNA Nanolevers (Dynamic Biosensors GmbH), target EpCAM to cNL-A96, complementary to DNA with green fluorophore, and target MSLN to cNL-B96, complementary to DNA with red fluorophore. All measurements were performed in running buffer HE140 (10 mM Hepes, 140 mM NaCl, 0.05% Tween20, 50 μM EDTA, 50 μM EGTA, pH7.4) at room temperature.

Kinetic measurements of a multitargeting antigen-binding molecule, i.e. the dual targeting EpCAMxMSLN HLE BiTE® molecule (SEQ ID NO: 3704) were performed in fluorescence proximity sensing mode using a standard kinetics assay from the switchBUILD software (Dynamic Biosensors GmbH) with several BiTE molecule concentrations. Therefore, the two DNA-linked antigens EpCAM and MSLN were mixed at an equal ratio and immobilized on the biochip to evaluate simultaneous binding of a dual targeting HLE BiTE® molecule to each antigen upon distinct detection of quenching of each fluorophore. Kinetic rate constants (K_{D}, kₒₙ and k_{off}) were calculated with the switchANALYSIS software (Dynamic Biosensors GmbH).

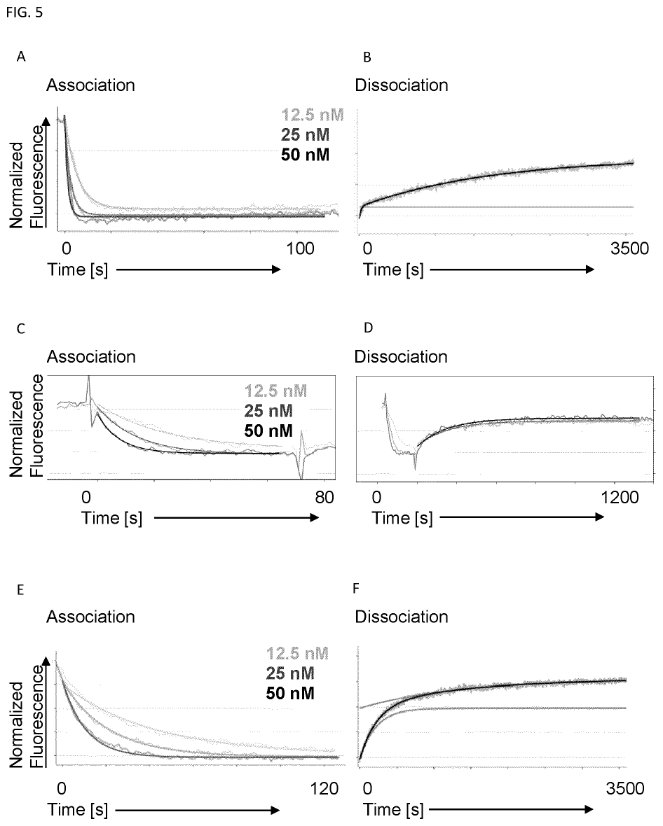

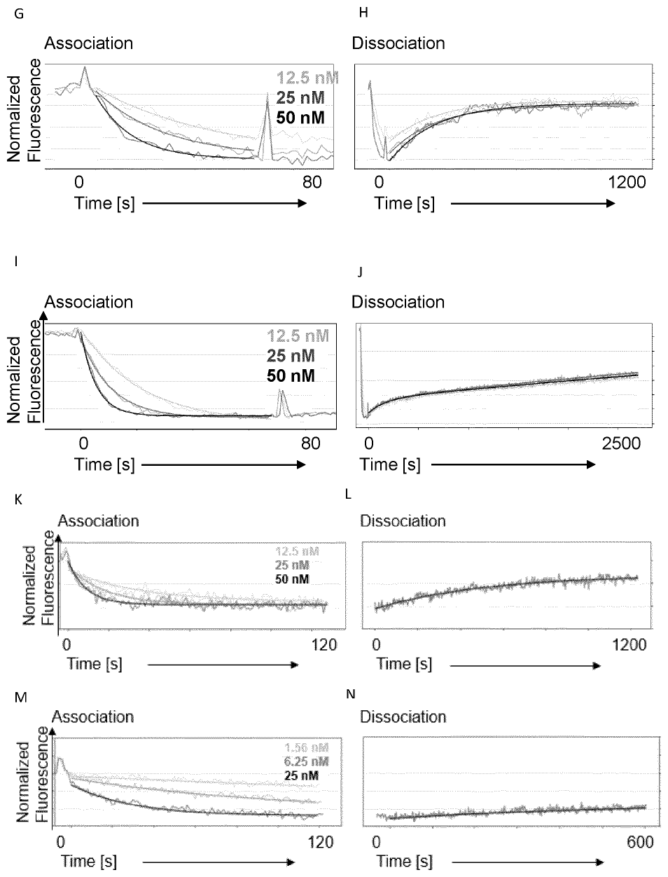

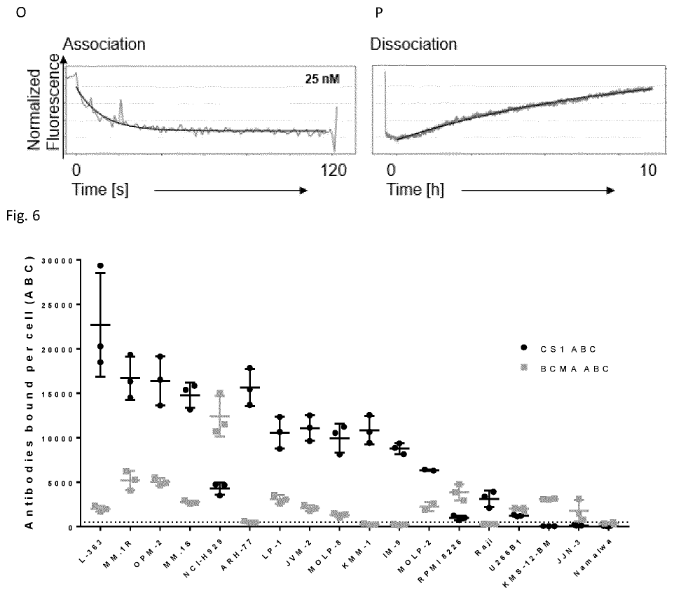

Results (FIG. 5A to F)

Binding kinetics of a dual targeting HLE BiTE® molecule to two antigens EpCAM and MSLN (SEQ ID NO: 3704) were determined on a DRX2 instrument, using a multi-purpose 96 bp dual color biochip immobilized with a mix of antigen EpCAM (target A) and MSLN (target B). It could be shown, that the dual targeting HLE BiTE® molecule binds to both antigens within one kinetic measurement. Furthermore, the presence of both antigens results in a biphasic dissociation of the HLE BiTE® molecule, with a faster dissociation k_{off,A} respectively k_{off,B} resulting from the binding to one target and a slower dissociation k_{off,AB} when bound to both antigens:

K_{D,A}=2.65±0.06 nM

k_{on,A}=1.72±0.03E+06M−1 s−1

k_{off,A}=4.55±0.08 E−03 s−1

K_{D,AB}=389±5 pM

k_{on,Ab}=1.87±0.03E+06M−1 s−1

k_{off,AB}=7.30±0.30E−04 s−1

K_{D,B}=4.51±0.05 nM

k_{on,B}=1.20±0.04E+07M−1 s−1

k_{off,B}=5.41±0.52 E−02 s−1

K_{D,AB}=39±1 pM

k_{on,AB}=1.50±0.04 E+07 M−1 s−1

k_{off,AB}=5.80±0.60 E−04 s−1

This leads to a higher overall binding strength, seen in the increase of KD values, KD,A 2.65 nM to KD,AB of 389 pM and KD,B 4.51 nM to KD,AB of 389 pM. This improvement of KD respectively the higher binding strength of the HLE BiTE® molecule, demonstrates the simultaneous binding of the two target binding domains.

### Example 3: Determination of Simultaneous Binding of CLL1 and FLT3 Target Binding Domains by Electrically Switchable Nanolever Measurement on Mixture of Targets

All Electrically switchable nanolever measurements were performed on a DRX2 instrument, using a bi-functional 96 bp dual color biochip (switchSENSE® by Dynamic Biosensors GmbH; Planegg, Germany). Prior to kinetic affinity experiments, target antigens C (CLL1) and D (FLT3) were conjugated to DNA nanolevers (Dynamic Biosensors GmbH). Target C to DNA nanolevers cNL-B42 and target D to DNA nanolevers cNL-B96. For dual binding assays, target D was additionally conjugated to cNL-B48 to present the two different targets simultaneously on one DNA nanolever.
Measurements were performed in running buffer HE140 (10 mM Hepes, 140 mM NaCl, 0.05% Tween20, 50 μM EDTA, 50 μM EGTA, pH7.4) at room temperature.

Kinetic measurements of the dual targeting BiTE molecule Y were performed in fluorescence proximity sensing mode using a standard kinetics assay from the switchBUILD software (Dynamic Biosensors GmbH) with several BiTE molecule concentrations. Kinetic rate constants (KD, kon and koff) were calculated with switchANALYSIS software (Dynamic Biosensors GmbH). For affinity measurements, antigen C (CLL1) and D (FLT3) were immobilized separately on the chip and binding of each target binding domain of the dual targeting BiTE molecule Y was evaluated based on the strength of fluorophore quenching. For dual binding assays, the two DNA-linked antigens C and D were mixed at a 1:1 ratio prior to immobilization on the chip and kinetic measurements were performed with dual targeting BiTE Molecule® (SEQ ID NO: 3736).

Results (FIG. 5G to J)

Binding kinetics to target antigen C and D of a dual targeting BiTE® molecule (SEQ ID NO: 3736) were determined on a DRX2 instrument, using a bi-functional 96 bp dual color biochip immobilized with either antigen C or D, or a 1:1 mix of both antigens. It could be shown that the dual targeting BiTE molecule binds to each antigen independently. On DNA nanolevers presenting both antigens, an increase in binding strength of the dual targeting BiTE® molecule could be measured, with an increase from KD,C 5.6 nM respectively KD,D 3.7 nM to KD,2 26.8 pM. This stronger binding of a dual targeting BiTE® molecule on a surface with both antigens demonstrates the simultaneous binding of the two target binding domains.

### Example 4: Determination of Simultaneous Binding of Target Binding Domains of Multitargeting CS1xBCMA Antigen-Binding Molecules

Simultaneous dual binding was determined by electrically switchable nanolever measurements which were performed on a DRX2 instrument, using a multi-purpose 96 bp dual color biochip (switchSENSE® by Dynamic Biosensors GmbH; Planegg, Germany). For measurements a DRX2 instrument and 96 bp multi-purpose biochips were used (Dynamic Biosensors GmbH). Prior to kinetic affinity experiments, target antigens CS1 and BCMA were conjugated to DNA nanolevers. Target CS1 was conjugated to DNA nanolevers cNL-B42 and target BCMA to DNA nanolevers cNL-A96. For avidity binding assays, target BCMA was additionally conjugated to cNL-B48 to present the two different targets simultaneously on one DNA nanolever.

Measurements were performed in running buffer HBS-EP (10 mM HEPES, 150 mM NaCl, 3 mM EDTA, 0.05% v/v Surfactant P20, pH 7.4) containing 0.05% Tween20 at room temperature. Kinetic measurements of a dual targeting CS1-BCMA bispecific antigen-binding molecule (SEQ ID NO: 1437) were performed in fluorescence proximity sensing mode using a standard kinetics assay from the switchBUILD software (Dynamic Biosensors GmbH) with several antibody concentrations. Kinetic rate constants (KD, kon and koff) were calculated with switchANALYSIS software (Dynamic Biosensors GmbH). For affinity measurements, antigens CS1 and BCMA were immobilized separately on a chip and binding of each target binding domain of the CS1-BCMA antigen-binding molecule was evaluated based on the strength of fluorophore quenching. For avidity binding assays, the two DNA-linked antigens CS1 and BCMA were mixed at a 1:1 ratio prior to immobilization on a chip and kinetic measurements were performed with the CS1-BCMA antigen-binding molecule.

Results (FIG. 5K to P)

Binding kinetics to target antigens CS1 and BCMA of the CS1-BCMA antigen-binding molecule were determined on a DRX2 instrument, using 96 bp multi-purpose biochips immobilized with either antigen CS1 or BCMA, or a 1:1 mix of both antigens.

Binding kinetics to target antigen CS1 K_{D,CS1}=892±60 pM

k_{on,CS1}=2.16±0.11 E+6 M−1 s−1

k_{off,CS1}=1.93±0.08 E−3 s−1

Binding kinetics to target antigen BCMA K_{D,BCMA}=1.27±0.30 nM

k_{on,BCMA}=1.30±0.08 E+6 M−1 s−1

k_{off,BCMA}=1.65±0.38 E−3 s−1

Binding kinetics to target antigens CS1 and BCMA K_{D,CS1BCMA}=10.3±0.80 pM

k_{on,CS1BCMA}=3.40±0.27 E+6 M−1 s−1

k_{off,CS1BCMA}=3.50±0.02 E−5 s−1

It could be shown that the CS1-BCMA antigen-binding molecule binds to each antigen independently. On DNA nanolevers presenting both antigens, an increase in binding strength of the CS1-BCMA T-cell engager molecule 1 could be measured, with an increase from K_{D,CS1} 892 pM respectively K_{D,BCMA}1.27 nM to K_{D,CS1BCMA} 10.3 pM. koff values changed from koff,CS1 1.93 E−3 s−1 respectively k_{off,BCMA} 1.65 E−3 s−1 to k_{offCS1BCMA} 3.50 E−5 s−1. This stronger binding of the CS1-BCMA antigen-binding molecule on a surface with both antigens demonstrates the simultaneous binding of the two target binding domains.

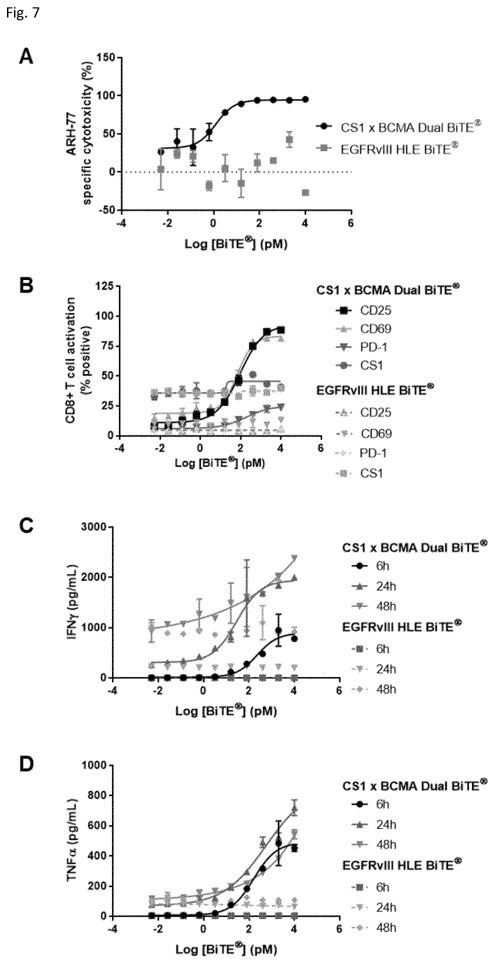

### Example 5: Re-Directed T Cell Lysis, Activation of T Cells, and Induction of Cytokines by the CS1 x BCMA Dual BiTE® Using ARH-77 Target Cells (Shown in FIG. 7A to C)

A) ARH-77 cells were co-cultured with human pan T cells at an effector-to-target (E:T) ratio of 10:1 for 48 hours in the absence or presence of the indicated BiTE® molecule over a dose range. Target cell lysis was measured by flow cytometry, as determined by loss of cell membrane integrity which was reflected by nuclear uptake of the SYTOX™ Blue dye in CFDA-SE-labeled target cells. The tested BiTE® molecules included the CS1 x BCMA Dual BiTE® (•) and the negative control EGFRvIII HLE BiTE® (▪).

B) Markers of activation were assessed in CD8 T cells by flow cytometry. ARH-77 cells were co-cultured with human pan T cells and the indicated BiTE® molecules as described in A). The percentage of CD8 T cells positive for each indicated marker at each concentration of BiTE® molecule was quantified. The antibody clone RPA-T8 was used to identify CD8 T cells. The antibody clone M-A251 was used to detect CD25 (▪ solid line, CS1 x BCMA Dual BiTE®; □ dashed line, EGFRvIII HLE BiTE®); the antibody clone FN50 was used to detect CD69 (▴ solid line, CS1 x BCMA Dual BiTE®; ▾ dashed line, EGFRvIII HLE BiTE®); the antibody clone EH12.2H7 was used to detect PD-1 (▾ solid line, CS1 x BCMA Dual BiTE®; ♦ dashed line, EGFRvIII HLE BiTE®); and the antibody clone 162.1 was used to detect CS1 (• solid line, CS1 x BCMA Dual BiTE®; ▪ dashed line, EGFRvIII HLE BiTE®).

The levels of C) IFNγ and D) TNFα released by T cells in response to BiTE® treatment were measured using the MSD® immunoassay. ARH-77 cells were co-cultured with human pan T cells and the CS1 x BCMA Dual BiTE® molecule as described in A), and cell supernatants were collected at 6 hours (• solid line, CS1 x BCMA Dual BiTE®; ▪ dashed line, EGFRvIII HLE BiTE®), 24 hours (▴ solid line, CS1 x BCMA Dual BiTE®; ▾ dashed line, EGFRvIII HLE BiTE®), and 48 hours (▾ solid line, CS1 x BCMA Dual BiTE®; ♦ dashed line, EGFRvIII HLE BiTE®).

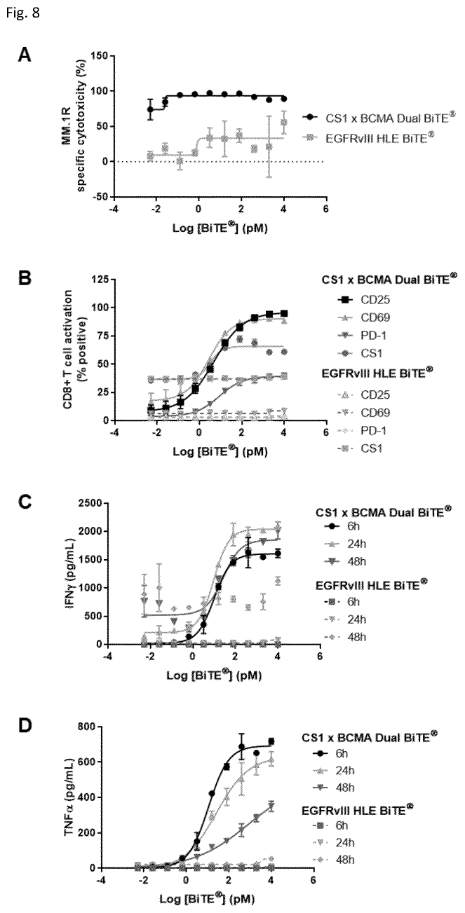

### Example 6: Re-Directed T Cell Lysis, Activation of T Cells, and Induction of Cytokines by the CS1 x BCMA Dual BiTE® Using MM.1R Target Cells (FIG. 8)

A) MM.1R cells were co-cultured with human pan T cells at an effector-to-target (E:T) ratio of 10:1 for 48 hours in the absence or presence of the indicated BiTE® molecule over a dose range. Target cell lysis was measured by flow cytometry, as determined by loss of cell membrane integrity which was reflected by nuclear uptake of the SYTOX™ Blue dye in CFDA-SE-labeled target cells. The tested BiTE® molecules included the CS1 x BCMA Dual BiTE® (•) and the negative control EGFRvIII HLE BiTE® (▪).
B) Markers of activation were assessed in CD8 T cells by flow cytometry. MM.1R cells were co-cultured with human pan T cells and the indicated BiTE® molecules as described in A). The percentage of CD8 T cells positive for each indicated marker at each concentration of BiTE® molecule was quantified. The antibody clone RPA-T8 was used to identify CD8 T cells. The antibody clone M-A251 was used to detect CD25 (▪ solid line, CS1 x BCMA Dual BiTE®; □ dashed line, EGFRvIII HLE BiTE®); the antibody clone FN50 was used to detect CD69 (▴ solid line, CS1 x BCMA Dual BiTE®; ▾ dashed line, EGFRvIII HLE BiTE®); the antibody clone EH12.2H7 was used to detect PD-1 (▾ solid line, CS1 x BCMA Dual BiTE®; ♦ dashed line, EGFRvIII HLE BiTE®); and the antibody clone 162.1 was used to detect CS1 (• solid line, CS1 x BCMA Dual BiTE®; ▪ dashed line, EGFRvIII HLE BiTE®).

The levels of C) IFNγ and D) TNFα released by T cells in response to BiTE® treatment were measured using the MSD® immunoassay. MM.1R cells were co-cultured with human pan T cells and the CS1 x BCMA Dual BiTE® molecule as described in A), and cell supernatants were collected at 6 hours (• solid line, CS1 x BCMA Dual BiTE®; ▪ dashed line, EGFRvIII HLE BiTE®), 24 hours (▴ solid line, CS1 x BCMA Dual BiTE®; ▾ dashed line, EGFRvIII HLE BiTE®), and 48 hours (▾ solid line, CS1 x BCMA Dual BiTE®; ♦ dashed line, EGFRvIII HLE BiTE®).

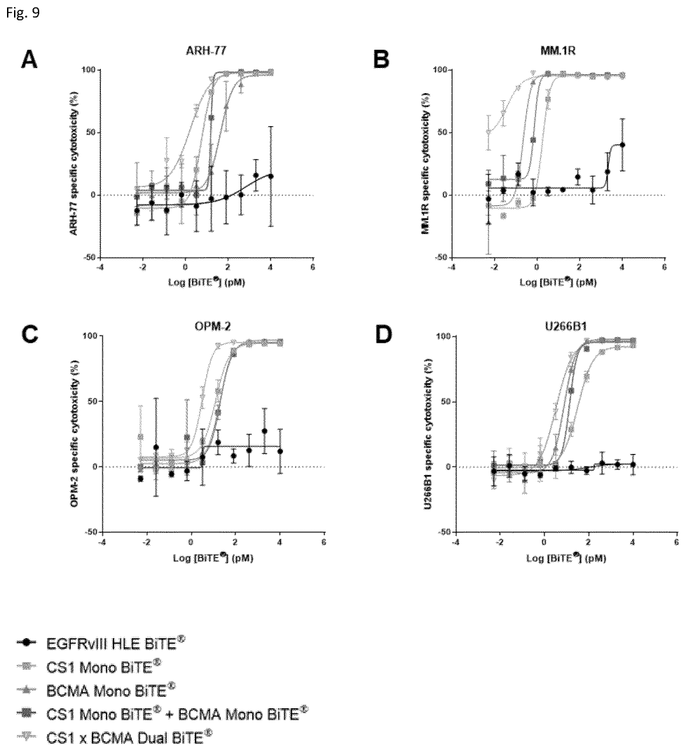

### Example 7: Re-Directed T Cell Lysis by the CS1 x BCMA Dual BiTE®, CS1 Mono BiTE®, and BCMA Mono BiTE® Using ARH-77, MM.1R, OPM-2, and U266B1 Target Cells (FIG. 9)

A) ARH-77, B) MM.1R, C) OPM-2, and D) U266B1 cells were co-cultured with human pan T cells at an effector-to-target (E:T) ratio of 10:1 for 48 hours in the absence or presence of the indicated BiTE® molecule over a dose range. Target cell lysis was measured by flow cytometry, as determined by loss of cell membrane integrity which was reflected by nuclear uptake of the TO-PRO™-3 iodide dye in CFDA-SE-labeled target cells. The tested BiTE® molecules included the CS1 x BCMA Dual BiTE® (□), the CS1 Mono BiTE® alone (▪), the BCMA Mono BiTE® alone (▴), the CS1 Mono BiTE® combined with the BCMA Mono BiTE® in equimolar ratio (▪), and the negative control EGFRvIII HLE BiTE® (•). The dual BiTE® molecule proved to be more active than the combination of mono BiTE® molecules.

TABLE 4
Re-directed T cell lysis by the CS1 × BCMA Dual BiTE ®, CS1 Mono
BiTE ®, and BCMA Mono BiTE® using ARH-77, MM.1R, OPM-2,
and U266B1 target cells

| | EGFRvIII HLE | CS1 Mono | BCMA Mono | CS1 Mono BiTE ® + | CS1 × BCMA |
|---|---|---|---|---|---|
| EC₅₀ (pm) | BiTE ® | BiTE ® | BiTE ® | BCMA Mono BiTE ® | Dual BiTE ® |
| ARH-77 | Undetermined | 5.50 | 42.39 | 15.23 | 1.63 |
| MM.1R | Undetermined | 1.75 | 0.22 | 0.73 | 0.04 |
| OPM-2 | Undetermined | 12.35 | 19.29 | 20.27 | 3.07 |
| U266B1 | Undetermined | 31.67 | 7.62 | 13.53 | 3.29 |

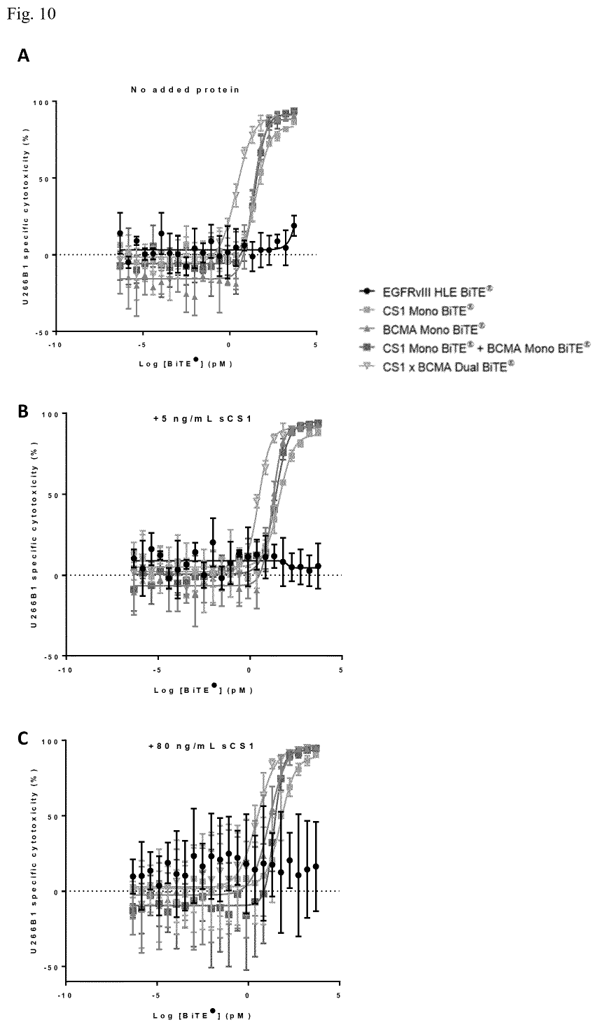

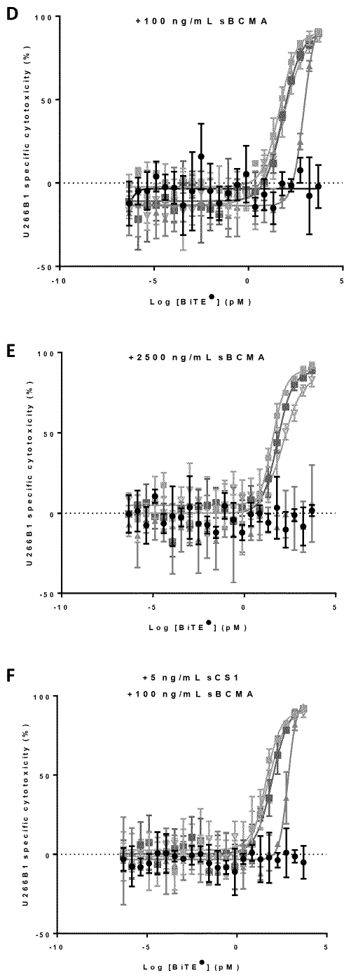

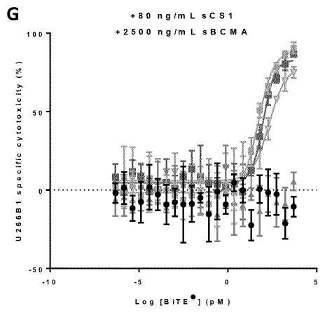

### Example 8: Re-Directed T Cell Lysis by the CS1 x BCMA Dual BiTE®, CS1 Mono BiTE®, and BCMA Mono BiTE® in the Presence of Soluble CS1 and/or Soluble BCMA Using U266B1 Target Cells (FIG. 10)

U266B1 cells were co-cultured with human pan T cells at an effector-to-target (E:T) ratio of 10:1 for 48 hours in the absence or presence of the indicated BiTE® molecule over a dose range. Target cells were labeled with luciferase, which was used to monitor target cell lysis. Recombinant soluble protein corresponding to the extracellular domain of either human CS1 (sCS1; 0, 5, or 80 ng/mL) or human BCMA (sBCMA; 0, 100, or 2500 ng/mL) was added to the assay mix either alone or in combination as indicated. The tested BiTE® molecules included the CS1 x BCMA Dual BiTE® (□), the CS1 Mono BiTE® alone (▪), the BCMA Mono BiTE® alone (▴), the CS1 Mono BiTE® combined with the BCMA Mono BiTE® in equimolar ratio (▪), and the negative control EGFRvIII HLE BiTE® (•). FIG. 10A) No added protein; B) 5 ng/mL sCS1; C) 80 ng/mL sCS1; D) 100 ng/mL sBCMA; E) 2500 ng/mL sBCMA; F) 5 ng/mL sCS1+100 ng/mL sBCMA; and G) 80 ng/mL sCS1+2500 ng/mL sBCMA.

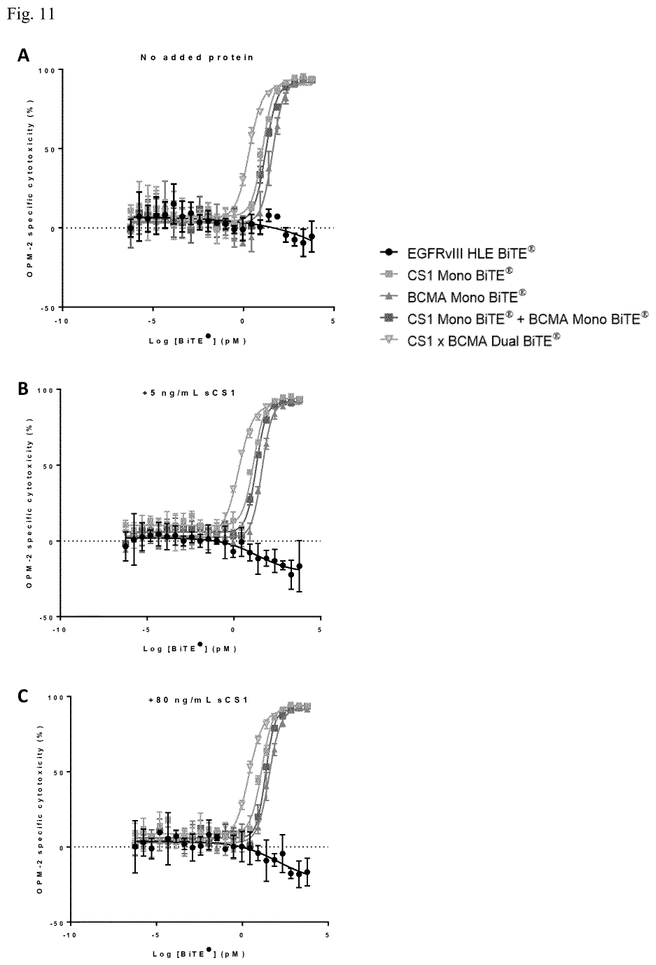

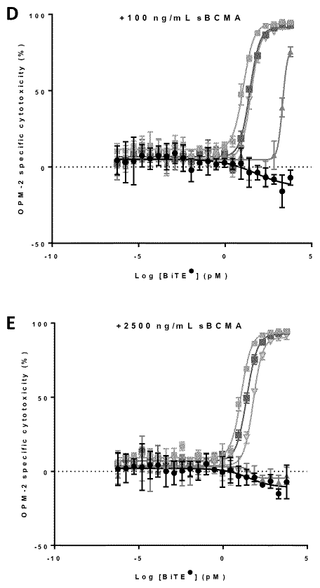

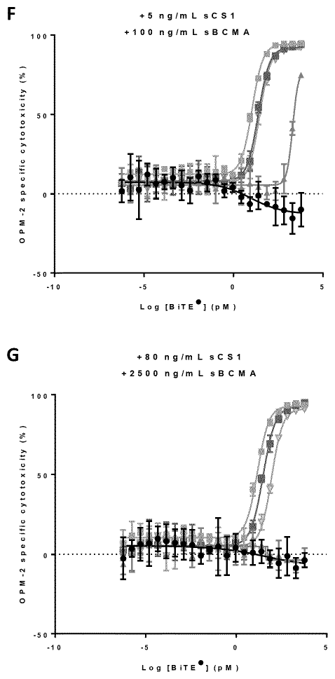

### Example 9: Re-Directed T Cell Lysis by the CS1 x BCMA Dual BiTE®, CS1 Mono Bite®, and BCMA Mono BiTE® in the Presence of Soluble CS1 and/or Soluble BCMA Using OPM-2 Target Cells (FIG. 11)

OPM-2 cells were co-cultured with human pan T cells at an effector-to-target (E:T) ratio of 10:1 for 48 hours in the absence or presence of the indicated BiTE® molecule over a dose range. Target cells were labeled with luciferase, which was used to monitor target cell lysis. Recombinant soluble protein corresponding to the extracellular domain of either human CS1 (sCS1; 0, 5, or 80 ng/mL) or human BCMA (sBCMA; 0, 100, or 2500 ng/mL) was added to the assay mix either alone or in combination as indicated. The tested BiTE® molecules included the CS1 x BCMA Dual BiTE® (□), the CS1 Mono BiTE® alone (▪), the BCMA Mono BiTE® alone (▴), the CS1 Mono BiTE® combined with the BCMA Mono BiTE® in equimolar ratio (▪), and the negative control EGFRvIII HLE BiTE® (•). FIG. 11A) No added protein; B) 5 ng/mL sCSI; C) 80 ng/mL sCSI; D) 100 ng/mL sBCMA; E) 2500 ng/mL sBCMA; F) 5 ng/mL sCS1+100 ng/mL sBCMA; and G) 80 ng/mL sCS1+2500 ng/mL sBCMA.

### Example 10: Re-Directed T Cell Lysis by the CS1 x BCMA Dual BiTE®, CS1 Mono BiTE®, and BCMA Mono BiTE® in the Presence of Soluble BCMA Using Three MM Target Cell Lines

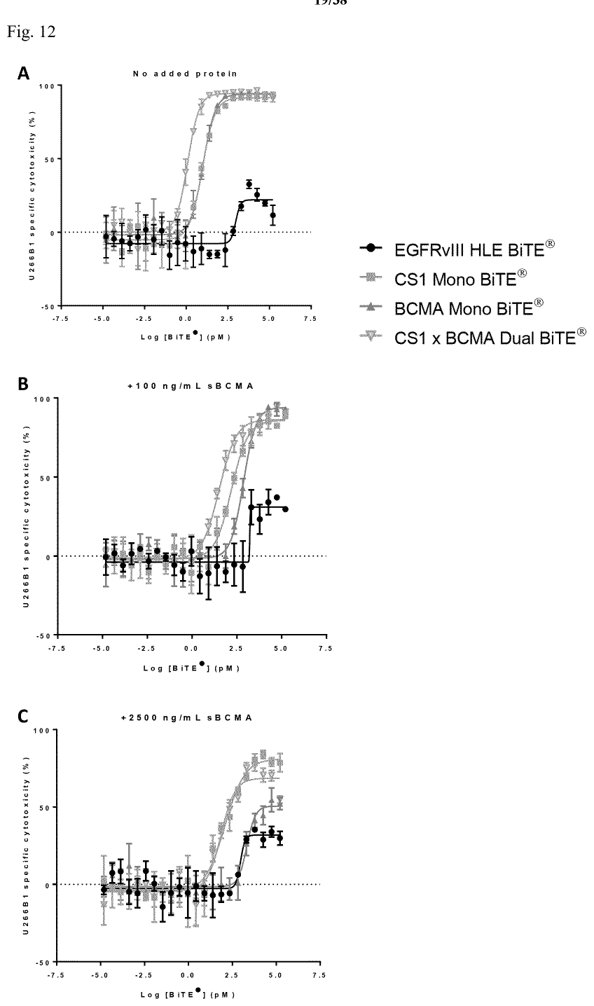

U266B1, NCI-H929, or OPM-2 cells were co-cultured with human pan T cells at an effector-to-target (E:T) ratio of 10:1 for 48 hours in the absence or presence of the indicated BiTE® molecule over a dose range. Target cells were labeled with luciferase, which was used to monitor target cell lysis. Recombinant soluble protein corresponding to the extracellular domain of human BCMA (sBCMA; 0, 100, or 2500 ng/mL) was added to the assay. The tested BiTE® molecules included the CS1 x BCMA Dual BiTE® (□), the CS1 Mono BiTE® alone (▪), the BCMA Mono BiTE® alone (▴), and the negative control EGFRvIII HLE BiTE® (•). FIG. 12A) U266B1 target cells+no added protein; B) U266B1 target cells+100 ng/mL sBCMA; C) U266B1 target cells+2500 ng/mL sBCMA; D) NCI-H929 target cells+no added protein; E) NCI-H929 target cells+100 ng/mL sBCMA; F) NCI-H929 target cells+2500 ng/mL sBCMA; G) OPM-2 target cells+no added protein; H) OPM-2 target cells+100 ng/mL sBCMA; and I) OPM-2 target cells+2500 ng/mL sBCMA.

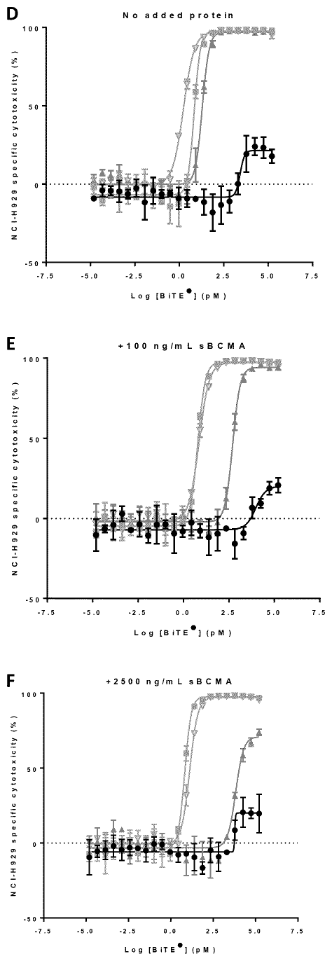

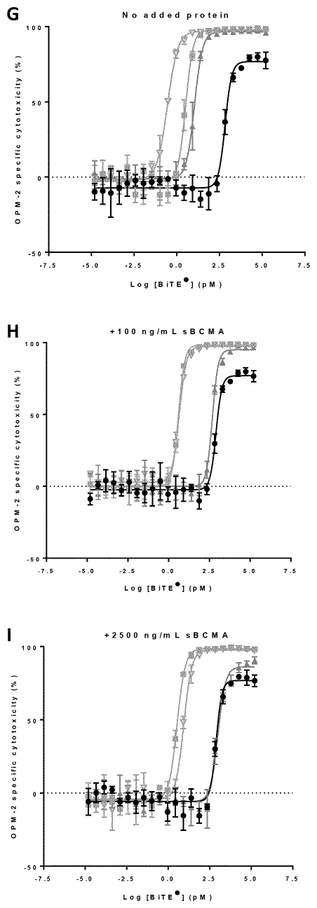

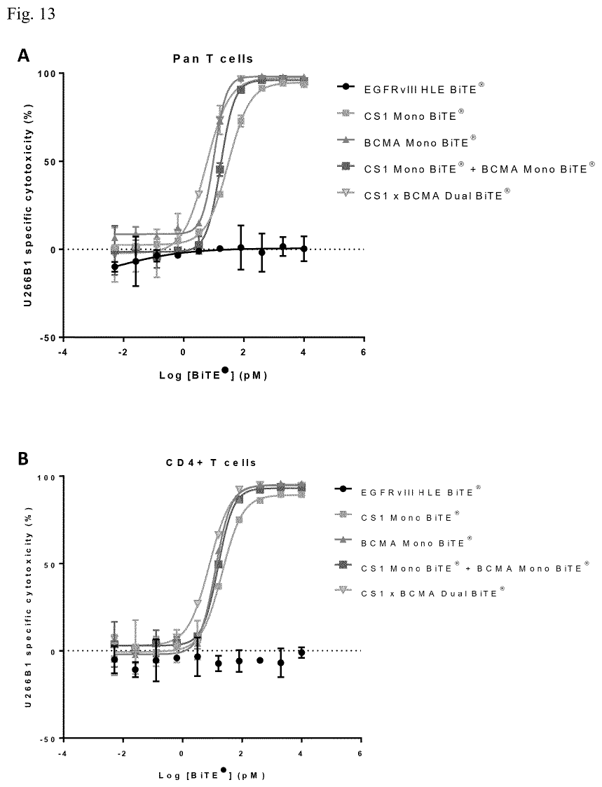

### Example 11: Re-Directed T Cell Lysis Using T Cell Sub-Populations by the CS1 x BCMA Dual BiTE®, CS1 Mono BiTE®, and BCMA Mono BiTE® Using U266B1 Target Cells (FIG. 13)

U266B1 cells were co-cultured with A) human pan T cells, B) purified CD4 T cells, or C) purified CD8 T cells at an effector-to-target (E:T) ratio of 10:1 for 48 hours in the absence or presence of the indicated BiTE® molecule over a dose range. Target cell lysis was measured by flow cytometry, as determined by loss of cell membrane integrity which was reflected by nuclear uptake of the TO-PRO™-3 iodide dye in CFDA-SE-labeled target cells. The tested BiTE® molecules included the CS1 x BCMA Dual BiTE® (□), the CS1 Mono BiTE® alone (▪), the BCMA Mono BiTE® alone (▴), the CS1 Mono BiTE® combined with the BCMA Mono BiTE® in equimolar ratio (▪), and the negative control EGFRvIII HLE BiTE® (•).

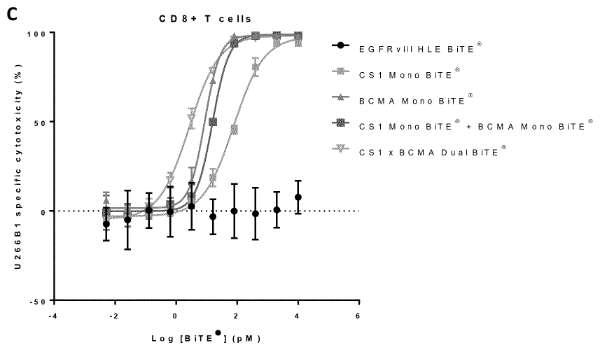

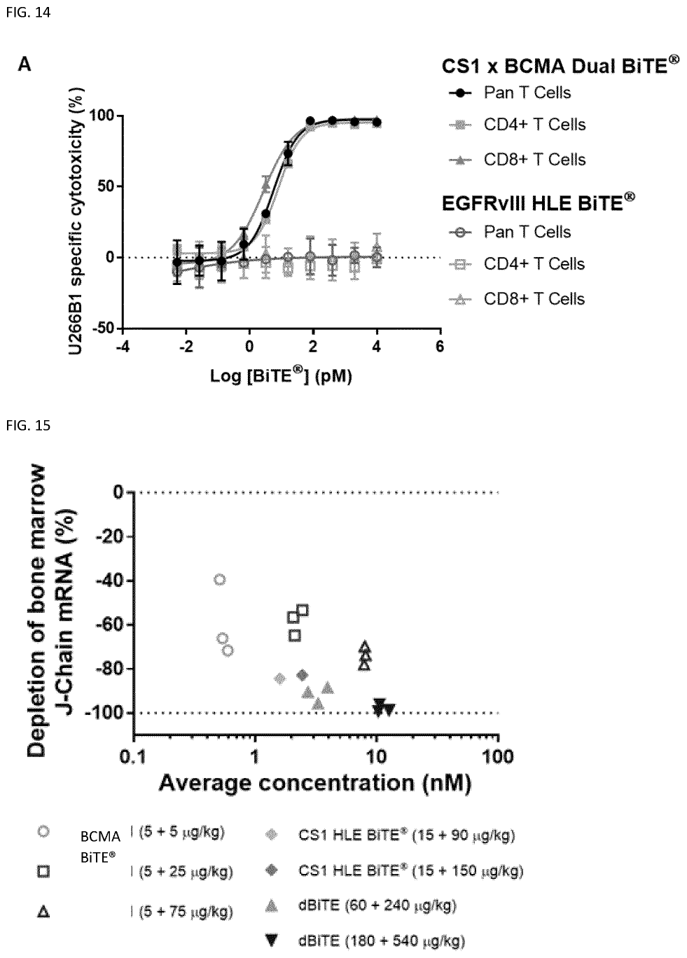

### Example 12: Re-Directed T Cell Lysis Using T Cell Sub-Populations by the CS1 x BCMA Dual BiTE® Using U266B1 Target Cells (FIG. 14)

A) U266B1 cells were co-cultured with human pan T cells (•, CS1 x BCMA Dual BiTE®; ∘, EGFRvIII HLE BiTE®), purified CD4 T cells (▪, CS1 x BCMA Dual BiTE®; □, EGFRvIII HLE BiTE®), or purified CD8 T cells (▴, CS1 x BCMA Dual BiTE®; □, EGFRvIII HLE BiTE®) at an effector-to-target (E:T) ratio of 10:1 for 48 hours in the absence or presence of the indicated BiTE® molecule over a dose range. Target cell lysis was measured by flow cytometry, as determined by loss of cell membrane integrity which was reflected by nuclear uptake of the TO-PRO™_3 iodide dye in CFDA-SE-labeled target cells.

### Example 13: Depletion of Bone Marrow in Cynomolgus Monkeys

Dual targeting CS1xBCMA antigen-binding molecule (SEQ ID NO: 1437) administered to cynomolgus monkeys is able to more completely deplete bone marrow as determined per J-Chain mRNA measurement than the respective mono BCMA and mono CS1 antigen-binding molecules according to the invention at the given doses which is desired in proliferative bone marrow diseases (FIG. 15).

### Example 14: Luciferase-Based Cytotoxicity Assay with Unstimulated Human PBMC Isolation of Effector Cells to Investigate Activity of Dual Targeting CD20xCD22 Antigen-Binding Molecules in Comparison to the Respective Mono Targeting Antigen-Binding Molecules

Human peripheral blood mononuclear cells (PBMC) were prepared by Ficoll density gradient centrifugation from enriched lymphocyte preparations (buffy coats), a side product of blood banks collecting blood for transfusions. Buffy coats were supplied by a local blood bank and PBMC were prepared on the day after blood collection. After Ficoll density centrifugation and extensive washes with Dulbecco's PBS (Gibco), remaining erythrocytes were removed from PBMC via incubation with erythrocyte lysis buffer (155 mM NH₄Cl, 10 mM KHCO₃, 100 μM EDTA). Remaining lymphocytes mainly encompass B and T lymphocytes, NK cells and monocytes. PBMC were kept in culture at 37° C./5% CO₂ in RPMI medium (Biochrom AG) with 10% FCS (Bio West).
Isolation of panT cells (CD3+) from PBMC
For isolation of panT cells, the Miltenyi human Pan T Cell Isolation Kit (#130-096-535) was used. PBMC were counted and centrifuged for 10 min at room temperature with 300×g. The supernatant was discarded and the cell pellet resuspended in MACS buffer (40 μL/10⁷ cells; Dulbecco's PBS+0.05% EDTA+0.5% FCS). 10 μl/10⁷ cells Pan T Cell Biotin-Antibody Cocktail were added, mixed and incubated at 4-8° C. for 5 min. After incubation 30 μl/10⁷ cells MACS buffer and 20 μl/10⁷ cells Pan T Cell MicroBead Cocktail were added, mixed and incubated at 4-8° C. for 10 min. PanT cells were then isolated using LS Columns (Milteny Biotec, #130-042-401). LS columns were placed in the magnetic field using a manual MACS Seperator. Colums were washed with 3 mL MACS buffer before loading cell suspension. After loading the cell suspension, the column was washed 3 times with 3 mL MACS buffer. The total flow-through containing unlabeled cells, representing enriched T cells, was collected. After isolation panT cells were washed with Dulbecco's PBS and adjusted to 1.2×10⁶ cells/mL and cultured in RPMI complete medium i.e. RPMI1640 (Biochrom AG, #FG1215) supplemented with 10% FBS (Bio West, #S1810), 1× non-essential amino acids (Biochrom AG, #K0293), 10 mM Hepes buffer (Biochrom AG, #L1613), 1 mM sodium pyruvate (Biochrom AG, #L0473) and 100 U/mL penicillin/streptomycin (Biochrom AG, #A2213) at 37° C., 5% CO₂ in an incubator until needed.

### Target Cell Preparation

Cells were harvested, spinned down and adjusted to 1.2×10⁵ cells/mL in complete RPMI medium. The vitality of cells was determined using Nucleocounter NC-250 (Chemometec) and Solution18 Dye containing Acridine Orange and DAPI (Chemometec).

### Luciferase Based Analysis

This assay was designed to quantify the lysis of target cells in the presence of serial dilutions of multi-specific antibody constructs. Equal volumes of Luciferase-positive target cells and effector cells (i.e., PBMC w/o CD14⁺; CD56⁺ cells) were mixed, resulting in an E:T cell ratio of 10:1. 42 μL of this suspension were transferred to each well of a 384-well plate. 8 μL of serial dilutions of the corresponding multi-specific antibody constructs and a negative control molecule (a CD3-based antibody construct recognizing an irrelevant target antigen) or RPMI complete medium as an additional negative control were added. The multi-specific antibody-mediated cytotoxic reaction proceeded for 48 hours in a 5% CO₂ humidified incubator. Then 25 μL substrate (Steady-Glo® Reagent, Promega) were transferred to the 384-well plate. Only living, Luciferase-positive cells react to the substrate and thus create a luminescence signal. Samples were measured with a SPARK microplate reader (TECAN) and analyzed by Spark Control Magellan software (TECAN).

### Percentage of Cytotoxicity was Calculated as Follows:

$$Cytoxicity [ \% ] = ( 1 - \frac{R L {U}_{Sample}}{R L {U}_{N e g a t i v e - C ontrol}} ) \times 100 \\ RLU = relative light units \\ Negative ‐ Control = cells without multi ‐ specific antibody construct$$

Using GraphPad Prism 7.04 software (Graph Pad Software, San Diego), the percentage of cytotoxicity was plotted against the corresponding multi-specific antibody construct concentrations. Dose response curves were analyzed with the four parametric logistic regression models for evaluation of sigmoid dose response curves with fixed hill slope and EC50 values were calculated.
Following target cell lines were used for the Luciferase-based cytotoxicity assay:
Effector Cells: panT Cells
Target cells: Ramos wt (CD20+ and CD22+)

### Legend:

CD20-CD22 T-cell engager molecule 1: CD20 99-E5 CC x CD22 28B7 N65S CC x I2C0 x scFc
CD20-CD22 T-cell engager molecule 2: CD20 99-E5 CC x CD22 02-A5 CC x I2C0 x scFc (SEQ ID NO: 3344)
CD20-CD22 T-cell engager molecule 3: CD20 20-C6 CC x CD22 28B7 N65S CC x I2C0 x scFc (SEQ ID NO: 1790)
CD20 T-cell engager molecule 1 (CD20 only binding): CD20 99-E5 CC x I2C0 x scFc (SEQ ID NO: 1598)
CD20 T-cell engager molecule 2 (CD20 only binding): CD20 20-C6 CC x I2C0 x scFc (SEQ ID NO: 1576)
CD22 T-cell engager molecule 1 (CD22 only binding): CD22 28-B7 N65S CC x I2C0 x scFc (SEQ ID NO: 1510)
CD22 T-cell engager molecule 2 (CD22 only binding): CD22 02-A5 CC x I2C0 x scFc (SEQ ID NO: 3345)

### Results:

TABLE 5
EC50 values in pM of CD20-CD22 T-cell engager molecules compared
to equimolar mixtures of CD20 and CD22 T-cell engager molecules
on Ramos wt cells

| | EC50 | | EC50 | |
|---|---|---|---|---|
| | [pM] | | [pM] | |
| CD20-CD22 T-cell | 0.077 | vs. | 305 | CD20 T-cell engager molecule |
| engager molecule 1 | | | | 1 and CD22 T-cell engager |
| | | | | molecule 1 |
| CD20-CD22 T-cell | 0.079 | vs. | 24 | CD20 T-cell engager molecule |
| engager molecule 2 | | | | 1 and CD22 T-cell engager |
| | | | | molecule 2 |
| CD20-CD22 T-cell | 0.039 | vs. | 105 | CD20 T-cell engager molecule |
| engager molecule 3 | | | | 2 and CD22 T-cell engager |
| | | | | molecule 1 |

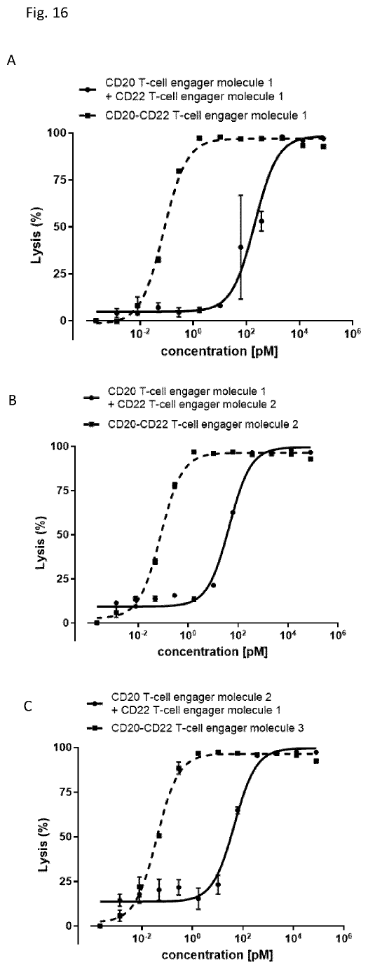

The tested dual targeting CD20-CD22 T-cell engager molecules 1, 2 and 3 (i.e. antigen-binding molecules) showed increased activity (lower EC50 values) on CD20/CD22 double positive Ramos wt cells compared to equimolar mixtures of corresponding CD20 and CD22 T-cell engager molecules on CD20/CD22 double positive Ramos wt cells (see FIG. 16 and Table 5).

### Example 15: Examination of Linker Length Flow Cytometry-Based Cytotoxicity Assay with Unstimulated panT Cells

### Isolation of Effector Cells

Human peripheral blood mononuclear cells (PBMC) were prepared by Ficoll density gradient centrifugation from enriched lymphocyte preparations (buffy coats), a side product of blood banks collecting blood for transfusions. Buffy coats were supplied by a local blood bank and PBMC were prepared on the day after blood collection. After Ficoll density centrifugation and extensive washes with Dulbecco's PBS (Gibco), remaining erythrocytes were removed from PBMC via incubation with erythrocyte lysis buffer (155 mM NH₄Cl, 10 mM KHCO₃, 100 μM EDTA). Remaining lymphocytes mainly encompass B and T lymphocytes, NK cells and monocytes. PBMC were kept in culture at 37° C./5% CO₂ in RPMI medium (Biochrom AG) with 10% FCS (Bio West).

Isolation of panT Cells (CD3+) from PBMC

For isolation of panT cells, the Miltenyi human Pan T Cell Isolation Kit (#130-096-535) was used. PBMC were counted and centrifuged for 10 min at room temperature with 300×g. The supernatant was discarded and the cell pellet resuspended in MACS buffer (40 μL/10⁷ cells; Dulbecco's PBS+0.05% EDTA+0.5% FCS). 10 μl/10⁷ cells Pan T Cell Biotin-Antibody Cocktail were added, mixed and incubated at 4-8° C. for 5 min. After incubation 30 μl/10⁷ cells MACS buffer and 20 μl/10⁷ cells Pan T Cell MicroBead Cocktail were added, mixed and incubated at 4-8° C. for 10 min. PanT cells were then isolated using LS Columns (Milteny Biotec, #130-042-401). LS columns were placed in the magnetic field using a manual MACS Seperator. Columns were washed with 3 mL MACS buffer before loading cell suspension. After loading the cell suspension, the column was washed 3 times with 3 mL MACS buffer. The total flow-through containing unlabeled cells, representing enriched T cells, was collected. After isolation panT cells were washed with Dulbecco's PBS and adjusted to 1.25×10⁶ cells/mL and cultured in RPMI complete medium i.e. RPMI1640 (Biochrom AG, #FG1215) supplemented with 10% FBS (Bio West, #S1810), 1× non-essential amino acids (Biochrom AG, #K0293), 10 mM Hepes buffer (Biochrom AG, #L1613), 1 mM sodium pyruvate (Biochrom AG, #L0473) and 100 U/mL penicillin/streptomycin (Biochrom AG, #A2213) at 37° C., 5% CO₂ in an incubator until needed.

Target Cell Labeling

For the analysis of cell lysis in flow cytometry assays, the fluorescent membrane dye DiOC₁₈ (DiO) (Thermo Fisher, #V22886) was used to label human- or macaque target transfected CHO cells as target cells and distinguish them from effector cells. Briefly, cells were harvested, washed once with PBS and adjusted to 10⁶ cell/mL in PBS containing 2% (v/v) FBS and the membrane dye DiO (5 μL/10⁶ cells). After incubation for 3 min at 37° C., cells were washed twice in complete RPMI medium and the cell number adjusted to 1.25×10⁵ cells/mL. The vitality of cells was determined using Nucleocounter NC-250 (Chemometec) and Solution18 Dye containing Acridine Orange and DAPI (Chemometec).

Flow Cytometry Based Analysis

This assay was designed to quantify the lysis of cyno or human target-transfected CHO cells in the presence of serial dilutions of bispecific antigen-binding molecules of the invention. Equal volumes of DiO-labeled target cells and effector cells (i.e. CD3⁺ panT cells) were mixed, resulting in an E:T cell ratio of 10:1. 160 μl of this suspension were transferred to each well of a 96-well plate. 40 μL of serial dilutions of the corresponding target(s) x CD3 antigen-binding molecules and a negative control bispecific (a CD3-based bispecific antibody construct recognizing an irrelevant target antigen) or RPMI complete medium as an additional negative control were added. The bispecific molecule-mediated cytotoxic reaction proceeded for 48 hours in a 7% CO₂ humidified incubator. Then cells were transferred to a new 96-well plate and loss of target cell membrane integrity was monitored by adding propidium iodide (PI) at a final concentration of 1 pg/mL. PI is a membrane impermeable dye that normally is excluded from viable cells, whereas dead cells take it up and become identifiable by fluorescent emission.
Samples were measured by flow cytometry on an iQue Plus (Intellicyt, now Sartorius) instrument and analyzed by Forecyt software (Intellicyt). Target cells were identified as DiO-positive cells. PI-negative target cells were classified as living target cells. Percentage of cytotoxicity was calculated according to the following formula:

$$Cytotoxicity [ \% ] = \frac{{n}_{dead target cells}}{{n}_{target cells}} \times 100 \\ n = number of events$$

Using GraphPad Prism 7.04 software (Graph Pad Software, San Diego), the percentage of cytotoxicity was plotted against the corresponding bispecific antibody construct concentrations. Dose response curves were analyzed with the four parametric logistic regression models for evaluation of sigmoid dose response curves with fixed hill slope and EC50 values were calculated.
Following target cell lines were used for the FACS-based cytotoxicity assay:
HCT 116-LUC wt (CDH3+ and MSLN+)
OPM-2 (BCMA+ and CS1+)
EOL-1 (CD123+ and FLT3+)

TABLE 6
Result for dual targeting BCMA-CSI T-cell engager molecules
(i.e. antigen-binding molecules) with different linkers, Effector cells:
panT cells Target cells: OPM-2

| | EC50 [pM] |
|---|---|
| BCMA-CS1 T-cell engager molecule 1 | 0.83 |
| BCMA-CS1 T-cell engager molecule 2 | 0.58 |
| BCMA-CS1 T-cell engager molecule 3 | 1.38 |

### Legend:

BCMA-CS1 T-cell engager molecule 1: CS PDL.12 LH CC x SG4S x BC A7 27-C4-G7 CC x I2C0 x scFc; with 6 AA linker (SEQ ID NO: 1437)
BCMA-CS1 T-cell engager molecule 2: CS PDL.12 LH CC x S(G4S)2 x BC A7 27-C4-G7 CC x I2C x scFc; with 11 AA linker (SEQ ID NO: 3761)
BCMA-CS1 T-cell engager molecule 3: CS PDL.12 LH CC x S(G4S)3 x BC A7 27-C4-G7 CC x I2C x scFc; with 16 AA linker (SEQ ID NO: 3762)
EGFRvIII T-cell engager molecule (non-binding): EGFRvIII CC x I2C0 x scFc

### Results for Dual Targeting BCMA-CS1 T-Cell Engager Molecules:

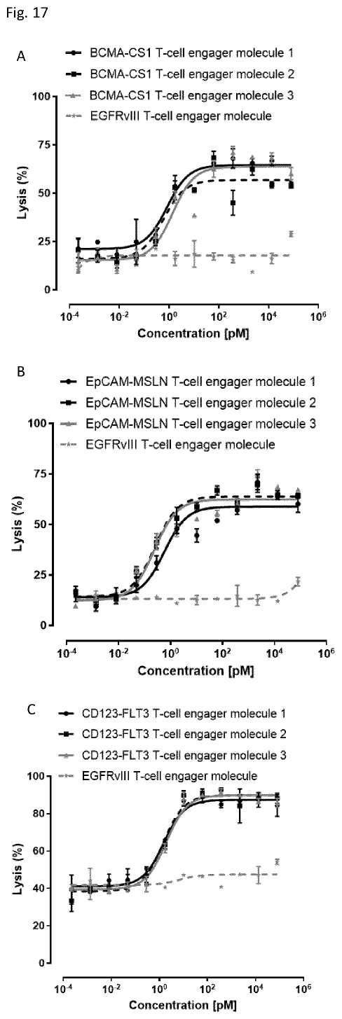

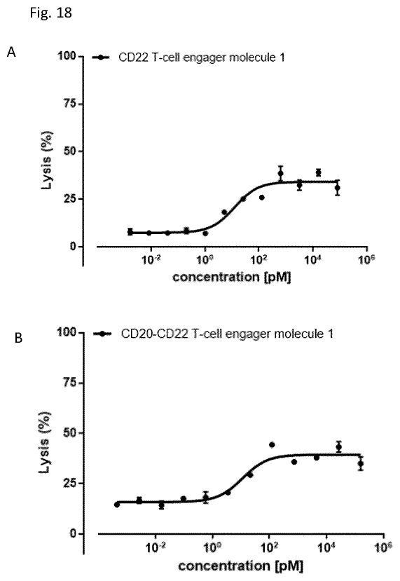

The tested BCMA-CS1 T-cell molecule 1 with 6 amino acid linker showed comparable EC50 values [pM] on OPM-2 cells as engager molecules 2 and 3 with longer linker variants (FIG. 17A).

TABLE 7
Results for dual targeting EpCAM-MSLN T cell engager molecules;
Effector cells: panT cells; Target cells: HCT 116 wt

| | EC50 [pM] |
|---|---|
| EpCAM-MSLN T-cell engager molecule 1 | 0.57 |
| EpCAM-MSLN T-cell engager molecule 2 | 0.25 |
| EpCAM-MSLN T-cell engager molecule 3 | 0.24 |

### Legend:

EpCAM-MSLN T-cell engager molecule 1: MSLN 4H6 CC x SG4S x EpCAM X1B x I2C0 x scFc with 6 AA linker (SEQ ID NO: 3705)
EpCAM-MSLN T-cell engager molecule 2: MSLN 4H6 CC x S(G4S)2 x EpCAM X1B x I2C0 x scFc with 11 AA linker (SEQ ID NO: 3710)
EpCAM-MSLN T-cell engager molecule 3: MSLN 4H6 CC x S(G4S)3 x EpCAM X1B x I2C0 x scFc with 16 AA linker (SEQ ID NO: 3711)
EGFRvIII T-cell engager molecule (non-binding): EGFRvIII CC x I2C0 x scFc

### Results for Dual Targeting EpCAM-MSLN T Cell Engager Molecules:

The tested EpCAM-MSLN T-cell engager molecules 2 and 3 with longer linker variants showed comparable EC50 values [pM] on HCT-116 cells as EpCAM-MSLN T-cell engager molecule 1 with original 6 amino acid linker (FIG. 17B).

TABLE 8
Results for dual targeting FLT3-CD123 T-cell engager
molecules: Effector cells: panT cells, Target cells: EOL-1

| | EC50 [pM] |
|---|---|
| CD123-FLT3 T-cell engager molecule 1 | 1.50 |
| CD123-FLT3 T-cell engager molecule 2 | 1.42 |
| CD123-FLT3 T-cell engager molecule 3 | 1.91 |

### Legend:

CD123-FLT3 T-cell engager molecule 1: FL 7-A8 CC x SG4S x CD123 24-B4-fNK CC x I2C0 x scFc; with 6 AA linker (SEQ ID NO: 3744)
CD123-FLT3 T-cell engager molecule 2: FL 7-A8 CC x S(G4S)2 x CD123 24-B4-fNK CC x I2C0 x scFc; with 11 AA linker (SEQ ID NO: 3747)
CD123-FLT3 T-cell engager molecule 3: FL 7-A8 CC x S(G4S)3 x CD123 24-B4-fNK CC x I2C0 x scFc; with 16 AA linker (SEQ ID NO: 3748)
EGFRvIII T-cell engager molecule (non-binding): EGFRvIII CC x I2C0 x scFc

### Results for Dual Targeting FLT3-CD123 T-Cell Engager Molecules:

The tested CD123-FLT3 T-cell engager molecules 2 and 3 with longer linker variants showed comparable EC50 values [pM] on OPM-2 cells as CD123-FLT3 T-cell engager molecule 1 with original 6 amino acid linker (FIG. 17C).

### Example 16: Comparability of Cytotoxic Activity Against CD22 Positive Cells by Both Mono and Dual Targeting Antigen-Binding Molecules

Flow Cytometry-Based Cytotoxicity Assay with Unstimulated panT Cells

### Isolation of Effector Cells

Human peripheral blood mononuclear cells (PBMC) were prepared by Ficoll density gradient centrifugation from enriched lymphocyte preparations (buffy coats), a side product of blood banks collecting blood for transfusions. Buffy coats were supplied by a local blood bank and PBMC were prepared on the day after blood collection. After Ficoll density centrifugation and extensive washes with Dulbecco's PBS (Gibco), remaining erythrocytes were removed from PBMC via incubation with erythrocyte lysis buffer (155 mM NH₄Cl, 10 mM KHCO₃, 100 μM EDTA). Remaining lymphocytes mainly encompass B and T lymphocytes, NK cells and monocytes. PBMC were kept in culture at 37° C./5% CO₂ in RPMI medium (Biochrom AG) with 10% FCS (Bio West).
Depletion of CD14⁺ and CD56⁺ cells
For depletion of CD14⁺ cells, human CD14 MicroBeads (Milteny Biotec, MACS, #130-050-201) were used, for depletion of NK cells human CD56 MicroBeads (MACS, #130-050-401). PBMC were counted and centrifuged for 10 min at room temperature with 300×g. The supernatant was discarded and the cell pellet resuspended in MACS isolation buffer (60 μL/10⁷ cells). CD14 MicroBeads and CD56 MicroBeads (20 μL/10⁷ cells) were added and incubated for 15 min at 4-8° C. The cells were washed with AutoMACS rinsing buffer (Milteny #130-091-222) (1-2 mL/10⁷ cells). After centrifugation (see above), supernatant was discarded and cells resuspended in MACS isolation buffer (500 μL/10⁸ cells). CD14/CD56 negative cells were then isolated using LS Columns (Milteny Biotec, #130-042-401). PBMC w/o CD14+/CD56+ cells were adjusted to 1.2×10⁶ cells/mL and cultured in RPMI complete medium i.e. RPMI1640 (Biochrom AG, #FG1215) supplemented with 10% FBS (Bio West, #S1810), 1× non-essential amino acids (Biochrom AG, #K0293), 10 mM Hepes buffer (Biochrom AG, #L1613), 1 mM sodium pyruvate (Biochrom AG, #L0473) and 100 U/mL penicillin/streptomycin (Biochrom AG, #A2213) at 37° C. in an incubator until needed.

### Target Cell Labeling

For the analysis of cell lysis in flow cytometry assays, the fluorescent membrane dye DiOC₁₈ (DiO) (Thermo Fisher, #V22886) was used to label human- or macaque target transfected CHO cells as target cells and distinguish them from effector cells. Briefly, cells were harvested, washed once with PBS and adjusted to 10⁶ cell/mL in PBS containing 2% (v/v) FBS and the membrane dye DiO (5 μL/10⁶ cells). After incubation for 3 min at 37° C., cells were washed twice in complete RPMI medium and the cell number adjusted to 1.25×10⁵ cells/mL. The vitality of cells was determined using Nucleocounter NC-250 (Chemometec) and Solution18 Dye containing Acridine Orange and DAPI (Chemometec).

### Flow Cytometry Based Analysis

This assay was designed to quantify the lysis of cyno or human target-transfected CHO cells in the presence of serial dilutions of bispecific antibody constructs. Equal volumes of DiO-labeled target cells and effector cells (i.e. CD3⁺ panT cells) were mixed, resulting in an E:T cell ratio of 10:1. 160 μl of this suspension were transferred to each well of a 96-well plate. 40 μL of serial dilutions of the corresponding target x CD3 bispecific antibody constructs and a negative control bispecific (a CD3-based bispecific antibody construct recognizing an irrelevant target antigen) or RPMI complete medium as an additional negative control were added. The bispecific antibody-mediated cytotoxic reaction proceeded for 48 hours in a 7% CO₂ humidified incubator. Then cells were transferred to a new 96-well plate and loss of target cell membrane integrity was monitored by adding propidium iodide (PI) at a final concentration of 1 μg/mL. PI is a membrane impermeable dye that normally is excluded from viable cells, whereas dead cells take it up and become identifiable by fluorescent emission.
Samples were measured by flow cytometry on an iQue Plus (Intellicyt, now Sartorius) instrument and analyzed by Forecyt software (Intellicyt). Target cells were identified as DiO-positive cells. PI-negative target cells were classified as living target cells. Percentage of cytotoxicity was calculated according to the following formula:

$$Cytotoxicity [ \% ] = \frac{{n}_{dead target cells}}{{n}_{target cells}} \times 100 \\ n = number of events$$

Using GraphPad Prism 7.04 software (Graph Pad Software, San Diego), the percentage of cytotoxicity was plotted against the corresponding bispecific antibody construct concentrations. Dose response curves were analyzed with the four parametric logistic regression models for evaluation of sigmoid dose response curves with fixed hill slope and EC50 values were calculated.
Following target cell lines were used for the FACS-based cytotoxicity assay:
Raji (CD20+ and CD22+)
Raji CRISPR CD20 #4 (CD20- and CD22+)

TABLE 9
Comparison of cytotoxicity of CD22 T-cell engager molecule 1 and
CD20-CD22 T-cell engager molecule 1 with Effector cells: human
unstimulated T cells, Target cells: Raji and Effector cells: human
unstimulated T cells, Target cells: Raji CRISPR CD20, respectively

| | EC50 [pM] |
|---|---|
| CD22 T-cell engager molecule 1 | 12.34 |
| CD20-CD22 T-cell engager molecule 1 | 11.1 |

### Legend:

CD22 T-cell engager molecule 1: CD22 28-B7 N65S CC x I2C0 x scFc (SEQ ID NO 1510)
CD20-CD22 T-cell engager molecule 1: CD20 20-C6 CC x CD22 28B7 N65S CC x I2C0 x scFc

### Results:

The tested CD22 T-cell engager molecule 1 showed comparable EC50 values [pM] on Raji cells (FIG. 18A) as CD20-CD22 T-cell engager molecule 1 on Raji CRISPR CD20 cells (FIG. 18B).

### Example 17 CD20 Epitope Mapping Epitope Clustering of Bispecific CD20 Antigen-Binding Molecules

### Construct Generation

The human CD20 protein extracellular region was divided into two parts: (1) extracellular loop 1 (ECL1), designated E1, and extracellular loop 2 (ECL2), designated E2. The extracellular loop 1 (E1) was further divided into two subparts, designated ElA and E1B. The extracellular loop 2 (E2) was further divided into four subparts, designated E2A, E2B, E2C and E2D.

TABLE 10
E1, E2 and the respective subparts E1A, E1B, E2A, E2B, E2C and
E2D have the following amino acid (aa) positions of the
human CD20 protein:

| Name | | |
|---|---|---|
| of | Amino acid | |
| Epitope | Region | Comment |
| E1 | aa 72 to 84 | Comprises E1A and E1B |
| E1A | aa 72 to 79 | IPAGIYAPI core epitope for Ofatumumab^{1 + 2}. |
| | | E1A comprises IPAGIYAP |
| E1B | aa 80 to 84 | PICVTV: ECL1 according to Uniprot P11836, |
| | | E1B comprises ICVTV |
| E2 | aa 142 to 188 | ECL2 according to Uniprot P11836 |
| E2A | aa 142 to 161 | FLKMESLNFIRAHT: core epitope of |
| | | Ofatumumab^{1 + 2}. E2A comprises: |
| | | KISHFLKMESLNFIRAHTPY |
| E2B | aa 162 to 166 | |
| E2C | aa 167 to 175 | CEPANPSEK: epitope of Rituximab² |
| E2D | aa 176 to 188 | |

¹Teeling et al., J Immunol 2006; 177:362-371;
²Klein et al., mAbs 5:1, 22-33; Jan/Feb 2013

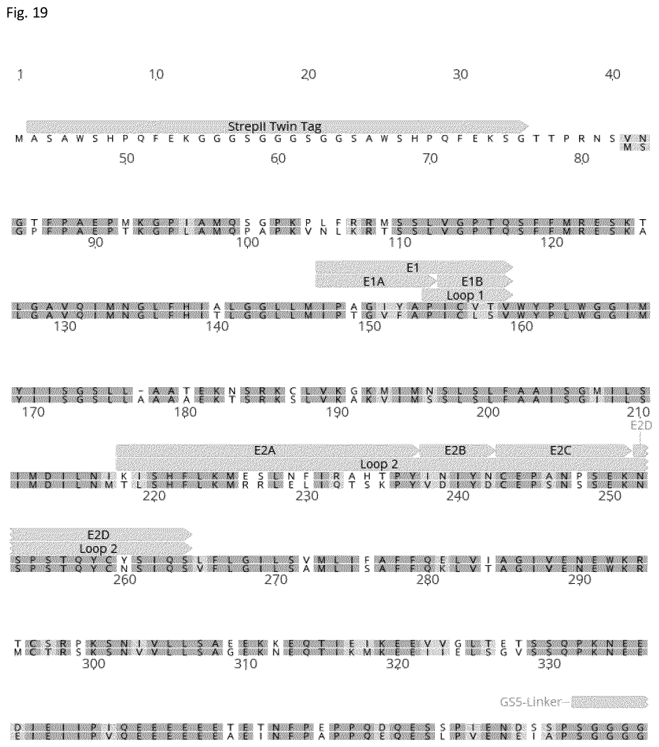

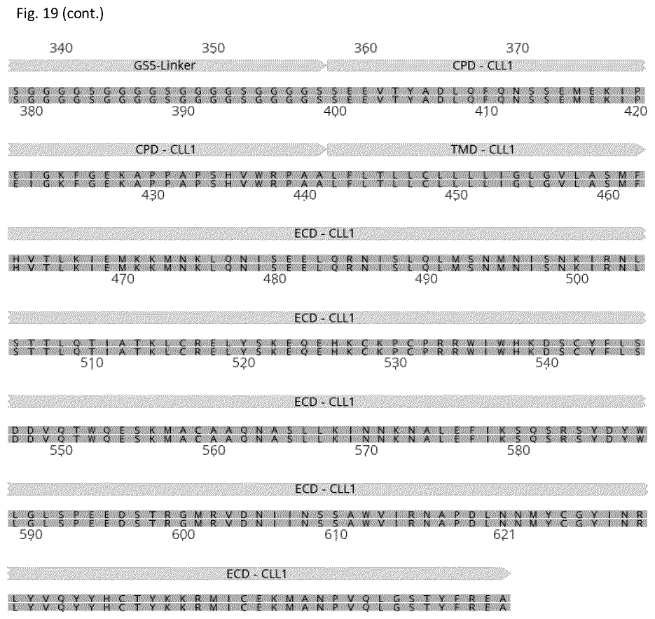

The human/mouse chimeric proteins were generated by replacing domains E1; E2 or the respective subparts of the human CD20 protein with the corresponding regions from mouse CD20 protein. At the N-terminal end the human CD20 and human/mouse chimeric CD20 proteins contain a Strep II Twin Tag that is of no significance for the assay described here. At the C-terminal end CLL1 was fused via a 5xGS-linker as an expression proof. The protein sequence of each of the constructs described above is depicted in FIG. 19. Deoxyribonucleic acid (DNA) sequences encoding either full-length human CD20xCLL1, full-length mouse CD20XCLL1 protein or human/mouse chimeric CD20xCLL1 proteins were each cloned into a pEFdhfr vector and stably transfected into CHO dihydrofolate reductase-negative (DHFR−) cells.

Transfection

CHO DHFR-cells (1×106) were transfected using 82 μl of Nucleofector Solution DNA Transfection Reagent combined with 18 μl Supplement 1 both components of the Amaxa Cell Line Nucleofector Kit V and 2 μg of DNA encoding either the human CD20xCLL1 protein, the mouse CD20xCLL1 protein or chimeric human/mouse CD20xCLL1 proteins according to manufacturer's protocol. Cells were grown in RPMI Medium with supplements for 24 hours. Selection of adherent-growing cells expressing human, mouse or chimeric human/mouse CD20xCLL1 protein by nucleoside deprivation was done after 24 hours and cells were cultured in HyClone Medium with Pen/Strep at 37⁰ in a humidified incubator.

Flow Cytometry

To verify expression of the human CD20xCLL1 protein, the mouse CD20xCLL1 protein or chimeric human/mouse CD20xCLL1 proteins on stably transfected CHO, cells were incubated with 5 μg/mL of an anti-human CLL1 antibody (R&D Systems, clone 687317) and 1:100 dilution of PE-labeled anti mouse Fey secondary antibody (Jackson 115-116-071). Three different CD20 antibodies were used as a control. The human CD20xCLL1 protein, the mouse CD20xCLL1 protein or chimeric human/mouse CD20xCLL1 proteins were stained with 5 μg/ml of CD20 antibodies, clone MEM-97 (Abcam, ab8237) and clone B9E9 (Thermo Fisher, MA1-7636). CD20 antibody clone B-H2O (ab46892) was incubated with a 1:10 dilution. Binding of CD20 antibodies was detected with a 1:100 dilution of a PE-labeled anti-mouse Fey antibody (Jackson, 115-116-071). Expression of the mouse CD20xCLL1 protein on CHO cells was verified with 5 μg/ml of a PE-labeled anti-mouse CD20 antibody, clone SA275A11 (BioLegend, 150409).

To evaluate binding of several CD20 T-cell engager molecules to proteins expressed on the transfected cells, cells were incubated with 5 μg/mL of the respective T-cell engager molecules. Binding of these CD20-T cell engager molecules was detected using a 1:50 dilution of aPE-labeled anti-human Fey antibody. All antibodies were diluted in PBS with 2% FBS and all incubations were performed at 4° C. for 30 minutes. Washes were done using PBS with 2% FBS and the final suspension buffer prior to FACS analysis was also PBS with 2% FBS. Antibody binding was detected using a BD FACSCanto® II flow cytometer or with an Intellicyte IQe®. Changes in mean fluorescence were analyzed with BD FACSDiva®, v8.1, ForeCyt® and FlowJo®. Loss of the binding to the various human/mouse chimeric CD20 proteins was reflected as a decrease in signal detected by flow cytometry.

### Results

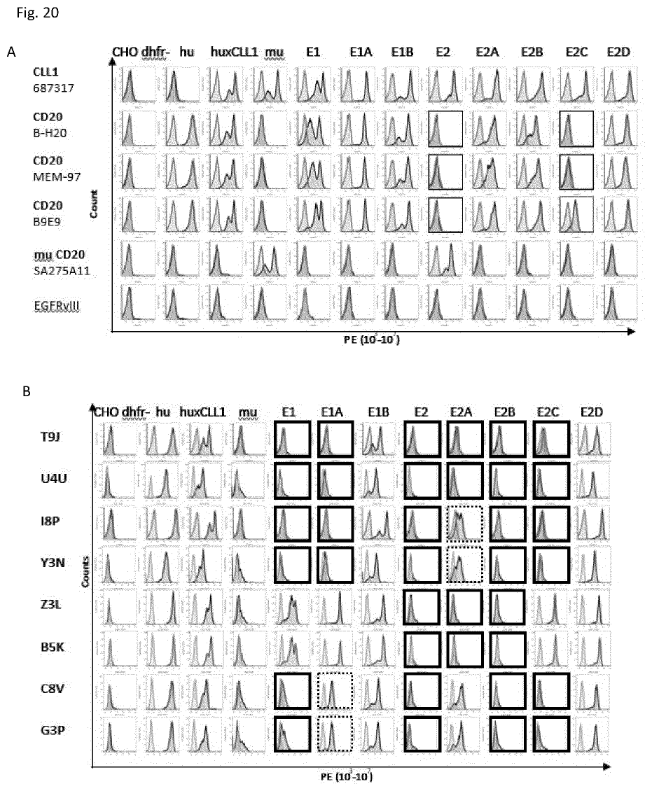

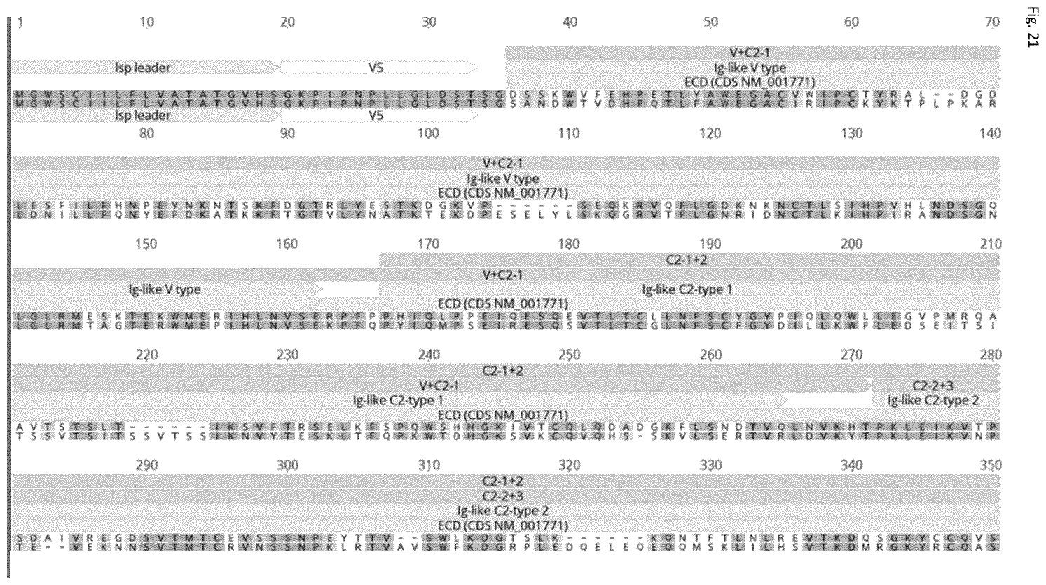

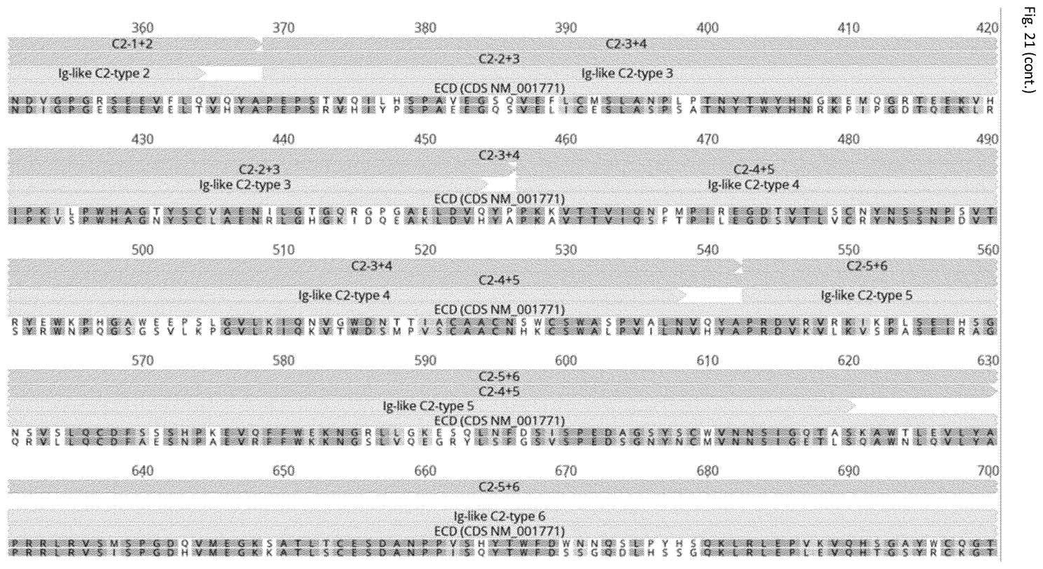

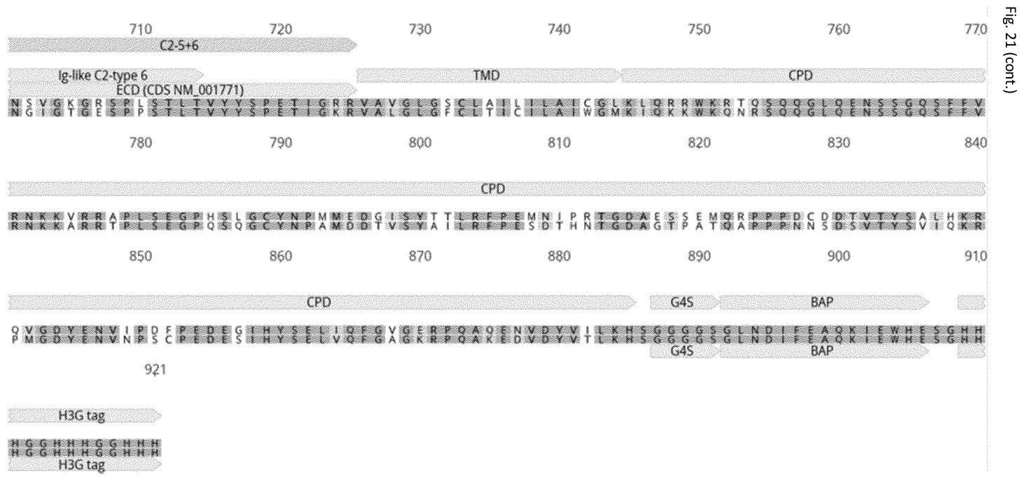

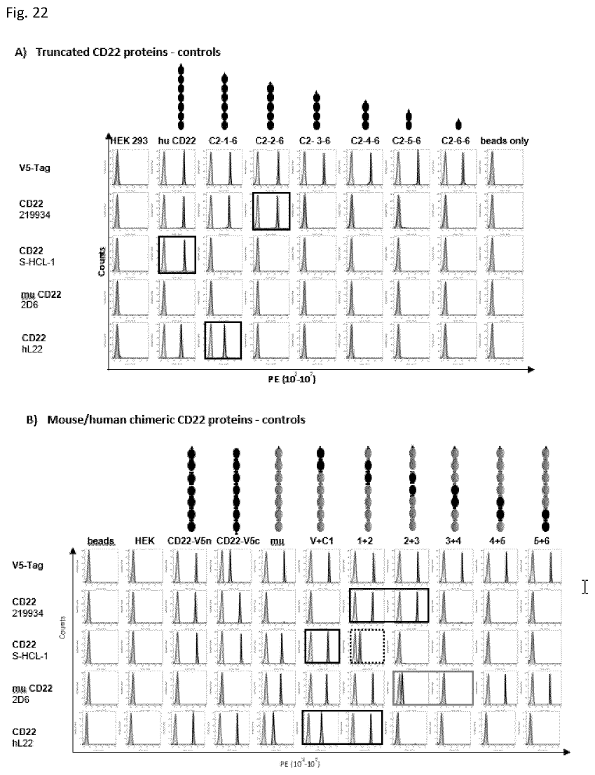

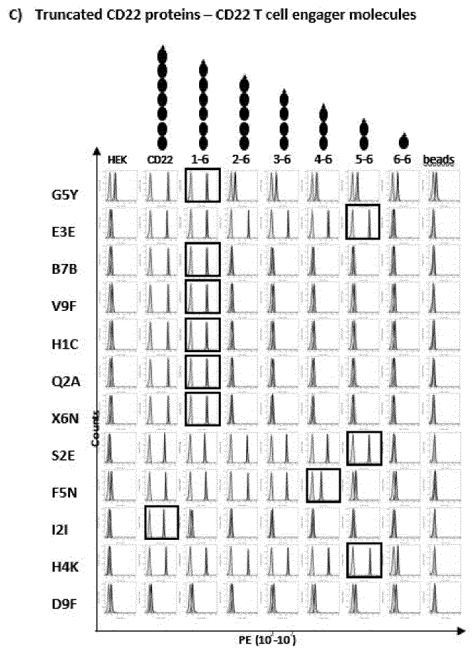

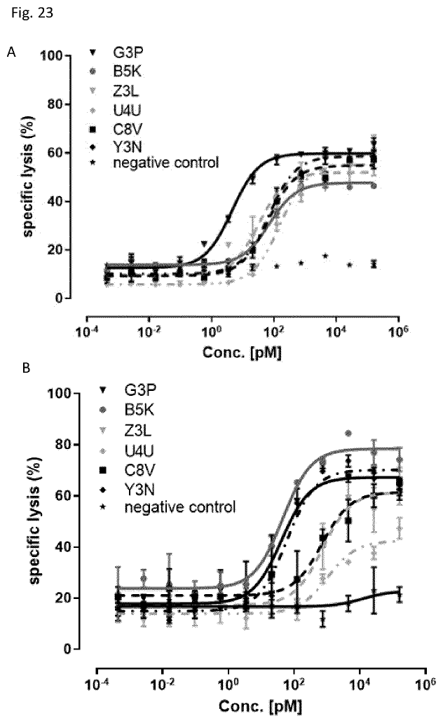

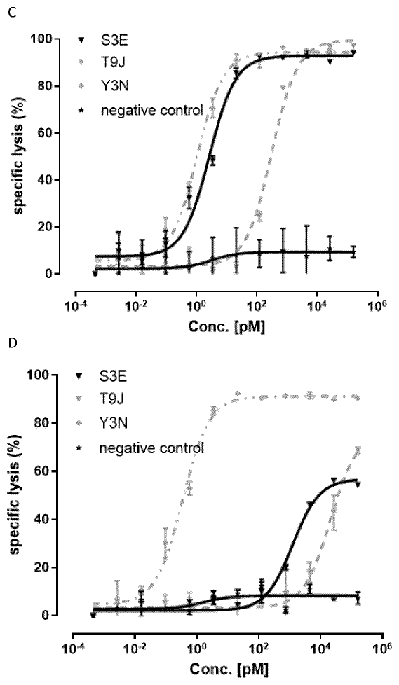

FIG. 19 depicts alignments of CD20xCLL1 protein sequences of human and mouse with epitope sections. The extracellular loop 1 (ECL1) of CD20 protein was designated E1, extracellular loop 2 (ECL2) was designated E2. For more refined epitope mapping, the extracellular loop 1 (ECL1) was further divided into ElA and E1B subparts and extracellular loop 2 (ECL2) was further divided into the subparts E2A, E2B, E2C or E2D.
For the epitope clustering, chimeric human/mouse CD20 proteins were generated in which regions of human CD20 protein were replaced with the corresponding regions from mouse CD20 protein. Because CD20 T cell engagers as disclosed herein (FIG. 20B) and known anti-human CD20 antibodies (FIG. 20A) do not bind mouse CD20 protein, the binding epitope region can be identified by systematically replacing sections of the human protein with the mouse protein (human/mouse CD20 chimeras) and determining which chimera is no longer recognized by the CD20 T-cell engagers or CD20 antibodies.

Human CD20, mouse CD20 and chimeric human/mouse CD20 proteins were stably expressed in CHO cells and binding of CD20-T Cell engager molecules (FIG. 20B) and known anti-human CD20 and anti-mouse CD20 antibodies for comparison (FIG. 20A) to surface-expressed proteins was assessed by flow cytometry (FIG. 20). Anti-human CD20 or anti-mouse CD20 antibodies were used to detect human and mouse CD20 in stably transfected CHO cells, respectively. Analysis of CD20-T cell engager binding to full length human and mouse CD20 and chimeric human/mouse CD20 proteins is described in Table 10 and FIG. 20B.

CD20 antigen-binding molecules of the present invention bound to cells expressing full length human CD20 protein, indicating it recognized the human extracellular domain. CD20-T cell engagers did not bind to cells expressing full length mouse CD20 protein, indicating it did not recognize the mouse extracellular domain. When binding to the domain-swapped proteins was evaluated, CD20-T cell engagers showed a variety of different binding patterns (Table 11 and FIG. 20). E.g. Z3L and B5K bound to human/mouse chimeric CD20 proteins containing the human E2, E2A and E2B domain. If the human E2, E2A or E2B domain was replaced with the mouse E2, E2A or E2B domain respectively, Z3L and B5K did not recognize the chimeric protein. Binding of Z3L and B5K was not affected by exchange of E1 or subparts of E1 (E1A and E1B), nor by exchange of E2C or E2D (subparts of E2).

Known CD20 antibodies B-H2O and MEM-97, for example, bound to human CD20 and human/mouse chimeric CD20 proteins containing the human E2 or subpart E2C. If the human E2 or subpart E2C was replaced with the mouse E2 or subpart E2C respectively, B-H2O and MEM-97 did not recognize the chimeric protein. Binding of B-H2O and MEM-97 was not affected by exchange of E1 or subparts of E1 (ElA and ElB), nor by exchange of E2A, E2B or E2D (subparts of E2).

### CONCLUSIONS

Sequence alignment of the CD20 protein shows each human sequence part (E1, E1A, E1B and E2, E2A, E2B, E2C and E2D) that was replaced with the corresponding mouse sequence and which amino acids differ between the two species.

CHO cells were transfected with human/mouse chimeric constructs and binding was assessed by flow cytometry. Control (secondary antibody only) is shown in light grey and CD20 antibody or CD20 T cell engager molecule binding is shown in darker grey. For the chimeric human/mouse CD20 proteins, human CD20 sequences for each epitope region (E1, E1A, E1B and E2, E2A, E2B, E2C, E2D) were individually replaced by mouse sequences. Each epitope region refers to the mouse portion of the chimeric molecule; e.g., E1 is a chimeric molecule for which the human E1 sequence has been replaced by the mouse E1 sequence, while the remaining sequence is human. Control anti-human CLL1 antibody (clone 687317) binding was used to verify expression of constructs. CD20-T cell engagers binds human, but not mouse full-length CD20 protein on CHO stable cell lines and show distintive binding patterns.

Hu: human CD20, huxCLL1: human CD20xCLL1, mu: mouse CD20, E1-E2D: human/mouse CD20 chimeric proteins.

TABLE 11
Epitope binding characteristics of representative CD20 and/or
CD22 antigen-binding molecules as disclosed herein

| Id | Molecule denomination | Epitope Cluster |
|---|---|---|
| T9J | CD20 43-B11 CC × I2C0 × scFc | E1A/E2ABC |
| U4U | CD20 97-H8_CC × CD22 77-A9 CC × | E1-A/E2-ABC |
| | I2C0 × scFc | |
| I8P | CD20 93-F7 CC × I2C0 × scFc | E1A/E2(A)BC |
| Y3N | CD20 01-C6-4_CC × CD22 28-B7 N655 | E1-AE2-(A)BC |
| | CC × I2C0 × scFc | |
| Z3L | CD22 29-H3 CC × CD20_29-F5_CC × | E2-AB |
| | I2C0 × scFc | |
| B5K | CD22 11-C3 CC × CD20_29-F5_CC × | E2-AB |
| | I2C0 × scFc | |
| C8V | CD22 29-H3 CC × CD20_99-G2_CC × | E1 (A)/E2-BC |
| | I2C0 × scFc | |
| G3P | CD20 99-E5_CC × CD22 28-B7 N655 | E1-(A)/E2-BC |
| | CC × I2C0 × scFc | |

### Example 18 Cytotoxic Activity Testing of Selected CD20 and/or CD22 Antigen-Binding Molecules

FACS based cytotoxicity assays testing CD2FxCD22 antigen-binding molecules U4U, Z3L, G3P, Y3N, B35K and C8V as disclosed herein on target cells CHO ff/Luc pCMV/hu orl CD22 vi pEFDHFR with Effector cells unstim. PBMC #263 (E:T-Ratio 50.000:5.000 S100 μl) in RPMI plus+10% FCS at a starting concentration of 160 nM and a dilution of 1:6 on F-bottom plate, on target cells CHO huCD2 pEFDHFR/ffLuc pCMV with Effector cells unstim. PBMC #263 (E:T-Ratio 50.000:5.000 S100 μl) in RPMI plus+10% FCS at a starting concentration of 160 nM and a dilution of 1:6 on F-bottom plate, and LUC based cytotoxicity assays testing CD20xCD22 antigen-binding molecule Y3N and CD20 antigen-binding molecules T9J and S3 as disclosed herein on target cells huCHO CD20+ Effector cells unstim. PBMC #773 (E:T-Ratio 25.000:2.500 S50 μl) in RPMI plus+100% FCS at a starting concentration of 160 nM and a dilution of 1:6 on 384-well F-bottom plate, and on target cells huCHO CD22+ Effector cells unstim. PBMC #773 (E:T-Ratio 25.000:2.500 S50 μl) in RPMI plus+10% FCS at a starting concentration of 160 nM and a dilution of 1:6 on 384-well F-bottom plate.

TABLE 12
The results of the above-described assays are summarized in the
table below.

| | EC50 |
|---|---|
| | [pM] |
| CHO huCD20 pEFDHFR/ffLuc pCMV | |
| CD20 97-H8_CC × CD22 77-A9 CC × I2C0 × scFc | 158.0 |
| U4U | |
| CD22 29-H3 CC × CD20_29-F5_CC × I2C0 × scFc | 31.7 |
| Z3L | |
| CD20 99-E5_CC × CD22 28-B7 N655 CC × I2C0 × scFc | 4.5 |
| G3P | |
| CD20 01-C6-4_CC × CD22 28-B7 N655 CC × I2C0 × scFc | 69.5 |
| Y3N | |
| CD22 11-C3 CC × CD20_29-F5_CC × I2C0 × scFc | 70.5 |
| B5K | |
| CD22 29-H3 CC × CD20_99-G2_CC × I2C0 × scFc | 64.8 |
| C8V | |
| CHO ff/Luc pCMV/hu orl CD22 v1 pEFDHFR | |
| CD20 97-H8_CC × CD22 77-A9 CC × I2C0 × scFc | 1043.6 |
| U4U | |
| CD22 29-H3 CC × CD20_29-F5_CC × I2C0 × scFc | 725.7 |
| Z3L | |
| CD20 99-E5_CC × CD22 28-B7 N655 CC × I2C0 × scFc | 41.0 |
| G3P | |
| CD20 01-C6-4_CC × CD22 28-B7 N655 CC × I2C0 × scFc | 60.0 |
| Y3N | |
| CD22 11-C3 CC × CD20_29-F5_CC × I2C0 × scFc | 44.9 |
| B5K | |
| CD22 29-H3 CC × CD20_99-G2_CC × I2C0 × scFc | 703.9 |
| C8V | |
| CHO huCD20 pEFDHFR/ffLuc pCMV | |
| CD20 01-C6-4 CC × CD22 28-B7 N655 CC × I2C0 × scFc Y3N | 1.0 |
| CD20 43-B11 CC × I2CO × scFc T9J | 311.1 |
| CD20 82-E2 CC × I2C0 × scFc S3E | 2.7 |
| CHO ff/Luc pCMV/hu orl CD22 v1 pEFDHFR | |
| CD20 01-C6-4 CC × CD22 28-B7 N655 CC × I2C0 × scFc Y3N | 0.3 |
| CD20 43-B11 CC × I2CO × scFcT9J | 20768.6 |
| CD20 82-E2 CC × I2CO × scFcS3E | 1238.9 |

TABLE 13
Correlation of epitope cluster and activity on CD20 positive target cells of different dual
targeting CD20 × CD22 antigen-binding molecules according to the present disclosure

| | Epitope | Epitope | | | | |
|---|---|---|---|---|---|---|
| | cluster | cluster | | | Activity | Activity |
| | parental | parental | experimental | Affinity | CHO | Raji |
| | Binder | Binder | sequence | huCD20 | huCD20 | CD20 |
| bispecifc dual targeting CD20 × CD22 molecule | CD20 | CD20 | identifier | [nM] | [pM] | [pM] |
| CD20 97-H8_CC × CD22 29-H3 CC × I2 CO × scFc | E1-A | E2-ABC | O4I | 70,51 | 112 | 529,0 |
| CD20 97-H8_CC × CD22 11-C3 CC × I2 CO × scFc | E1-A | E2-ABC | N5X | 124,14 | 160 | Flatline |
| CD20 97-H8_CC × CD22 02-C7 CC × I2 CO × scFc | E1-A | E2-ABC | M8Y | 133,93 | 464 | 607,3 |
| CD20 97-H8_CC × CD22 77-A9 CC × I2 CO × scFc | E1-A | E2-ABC | U4U | 84,20 | 158 | 642,4 |
| CD20 97-H8_CC × CD22 28-B7 N65S CC × I2 CO × | E1-A | E2-ABC | Q8Z | 69,68 | 63 | 195,7 |
| CD20 99-E5_CC × CD22 29-H3 CC × I2 CO × scFc | (E1)- | E2-BC | S3V | 76,41 | 5,4 | 8,4 |
| CD20 99-E5_CC × CD22 11-C3 CC × I2 CO × scFc | E1-(A) | E2-BC | Q8U | 103,25 | 10,3 | 14,7 |
| CD20 99-E5_CC × CD22 02-C7 CC × I2 CO × scFc | E1-(A) | E2-BC | X4F | 79,50 | 9,4 | 37,8 |
| CD20 99-E5_CC × CD22 77-A9 CC × I2 CO × scFc | E1-(A) | E2-BC | N9W | 195,30 | 7,3 | 15,9 |
| CD20 99-E5_CC × CD22 28-B7 CC × I2 CO × scFc | E1-(A) | E2-BC | G3P | 49,66 | 4,5 | 3,4 |
| CD20 01-C6-4_CC × CD22 29-H3 CC × I2 CO × scFc | E1-A | E2-(A)BC | Z1F | 122,50 | 48 | 75,3 |
| CD20 01-C6-4_CC × CD22 11-C3 CC × I2 CO × scFc | E1-A | E2-(A)BC | Z8O | 217,40 | 96 | 238,3 |
| CD20 01-C6-4_CC × CD22 02-C7 CC × I2 CO × scFc | E1-A | E2-(A)BC | I1B | 132,90 | 75 | 124,0 |
| CD20 01-C6-4_CC × CD22 77-A9 CC × I2 CO × scFc | E1-A | E2-(A)BC | T3W | 175,30 | 112 | 56,8 |
| CD20 01-C6-4_CC × CD22 28-B7 N65S CC × I2 CO | E1-A | E2-(A)BC | Y3N | 160,33 | 70 | 26,1 |
| CD20 13-D2_CC × CD22 29-H3 CC × I2 CO × scFc | E1-A | E2-(A)BC | D4A | 83,68 | 10,9 | 214,2 |
| CD20 13-D2_CC × CD22 11-C3 CC × I2 CO × scFc | E1-A | E2-(A)BC | G7N | 119,67 | 600 | 1520,4 |
| CD20 13-D2_CC × CD22 02-C7 CC × I2 CO × scFc | E1-A | E2-(A)BC | H7E | 85,87 | 226 | 275,4 |
| CD20 13-D2_CC × CD22 77-A9 CC × I2 CO × scFc | E1-A | E2-(A)BC | N1S | 158,90 | 398 | 1114,9 |
| CD20 13-D2_CC × CD22 28-B7 N65S CC × I2 CO × | E1-A | E2-(A)BC | T8X | 88,66 | 120 | 444,0 |
| CD20 20-D2_CC × CD22 29-H3 CC × I2 CO × scFc | | E2-AB | I5N | 0,76 | 0,8 | 2,0 |
| CD20 20-D2_CC × CD22 11-C3 CC × I2 CO × scFc | | E2-AB | F2I | 1,43 | 0,4 | 5,4 |
| CD20 20-D2_CC × CD22 02-C7 CC × I2 CO × scFc | | E2-AB | E5W | 0,98 | 0,2 | 2,5 |
| CD20 20-D2_CC × CD22 77-A9 CC × I2 CO × scFc | | E2-AB | N4P | 1,17 | 0,4 | 1,8 |
| CD20 20-D2_CC × CD22 28-B7 N65S CC × I2 CO × scFc | | E2-AB | B5W | 1,18 | 0,6 | 4,5 |

TABLE 14
Correlation of epitope cluster and activity on CD22 positive target cells of different dual
targeting CD20xCD22 antigen-binding molecules according to the present disclosure

| | Epitope | | | | |
|---|---|---|---|---|---|
| | cluster | experimental | Affinity | | |
| | parental | sequence | huCD22 | [symbol] ctivity CHO | Activity Raji |
| bispecific dual targeting CD20 x CD22 molecule | Binder CD22 | indentifier | [nM [symbol] | huCD22 [p [symbol] | CD22 [pM] |
| CD20 97-H8_CC × CD22 29-H3 CC × I2C0 × scFc | C2-1 | O4I | 0.18 | 112 | 21.3 |
| CD20 97-H8_CC × CD22 11-C3 CC × I2C0 × scFc | C2-5 | N5X | 15.92 | 160 | Flatline |
| CD20 97-H8_CC × CD22 02-C7 CC × I2C0 × scFc | C2-5 | M8Y | 8.99 | 464 | 2649.0 |
| CD20 97-H8_CC × CD22 77-A9 CC × I2C0 × scFc | C2-5 | U4U | 49.62 | 158 | Flatline |
| CD20 97-H8_CC × CD22 28-B7 N65S CC × I2C0 × | C2-1 | Q8Z | 0.14 | 63 | 14.3 |
| CD20 99-E5_CC × CD22 29-H3 CC × I2C0 × scFc | C2-1 | S3V | 0.27 | 5.4 | 46.7 |
| CD20 99-E5_CC × CD22 11-C3 CC × I2C0 × scFc | C2-5 | Q8U | 29.75 | 10.3 | Flatline |
| CD20 99-E5_CC × CD22 02-C7 CC × I2C0 × scFc | C2-5 | X3F | 8.88 | 9.4 | 1726.2 |
| CD20 99-E5_CC × CD22 77-A9 CC × I2C0 × scFc | C2-5 | N9W | 86.57 | 7.3 | Flatline |
| CD20 99-E5_CC × CD22 28-B7 N65S CC × I2C0 × [symbol] | C2-1 | G3P | 0.19 | 4.5 | 24.3 |
| CD20 01-C6-4_CC CD22 29-H3 CC × I2C0 × scFc | C2-1 | Z1F | 0.20 | 48 | 12.8 |
| CD20 01-C6-4_CC CD22 11-C3 CC × I2C0 × scFc | C2-5 | Z8O | 16.79 | 96 | Flatline |
| CD20 01-C6-4_CC CD22 02-C7 CC × I2C0 × scFc | C2-5 | I1B | 5.08 | 75 | 1673.9 |
| CD20 01-C6-4_CC CD22 77-A9 CC × I2C0 × scFc | C2-5 | T3W | 60.57 | 112 | Flatline |
| CD20 01-C6-4_CC CD22 28-B7 N65S CC × I2C0 | C2-1 | Y3N | 0.13 | 70 | 15.2 |
| CD20 13-D2_CC × CD22 29-H3 CC × I2C0 × scFc | C2-1 | D4A | 0.09 | 10.9 | 8.5 |
| CD20 13-D2_CC × CD22 11-C3 CC × I2C0 × scF | C2-5 | G7N | 12.66 | 600 | Flatline |
| CD20 13-D2_CC × CD22 02-C7 CC × I2C0 × scFc | C2-5 | H7N | 1.77 | 226 | 442.1 |
| CD20 13-D2_CC × CD22 77-A9 CC × I2C0 × scFc | C2-5 | N1S | 29.89 | 398 | Flatline |
| CD20 13-D2_CC × CD22 28-B7 N65S CC × I2C0 × | C2-1 | T8X | 0.14 | 230 | 14.2 |
| CD20 20-C6_CC × CD22 29-H3 CC × I2C0 × scFc | C2-1 | I5N | 0.15 | 0.9 | 12.0 |
| CD20 20-C6_CC × CD22 11-C3 CC × I2C0 × scFc | C2-5 | F2I | 3.22 | 0.4 | Flatline |
| CD20 20-C6_CC × CD22 02-C7 CC × I2C0 × scFc | C2-5 | E5W | 0.97 | 0.2 | 688.1 |
| CD20 20-C6_CC × CD22 77-A9 CC × I2C0 × scFc | C2-5 | N4P | 9.48 | 0.4 | Flatline |
| CD20 20-C6_CC × CD22 28-B7 N65S CC × I2C0 × [symbol] | C2-1 | BW5 | 0.13 | 0.6 | 11.1 |

[symbol]

indicates data missing or illegible when filed

Preferred molecules as disclosed herein are shown in Table 13. As it can be seen from Table 13, bispecific dual targeting CD20 x CD22 antigen-binding molecules which address the CD20 epitopes (i.) E1A and E2B and E2C or (ii.) E2A and E2B are particularly preferred in terms of superior on target cytotoxic activity. Preferred molecules as disclosed herein are shown in Table 14. As it can be seen from Table 14, bispecific dual targeting CD20 x CD22 antigen-binding molecules which address the CD22 epitope C2-1 are particularly preferred in terms of superior on target cytotoxic activity.

### Example 19: Selectivity Gap Through Dual Targeting Antigen-Binding Molecules in Different Formats

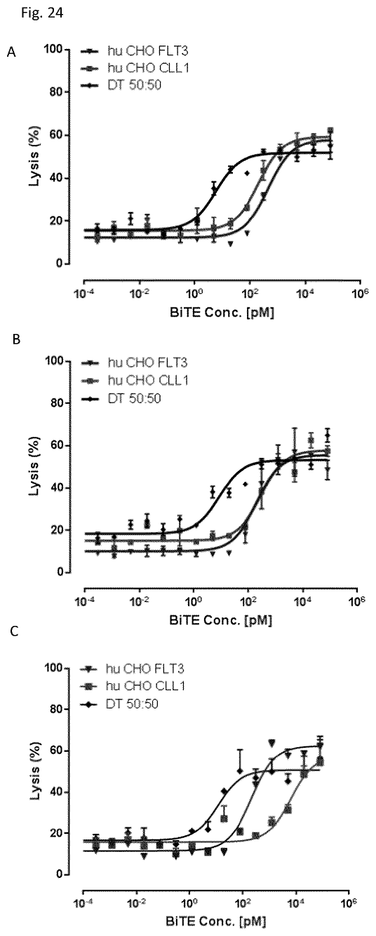

Three dual targeting CLL1xFLT3 antigen binding molecules with three different arrangements of domains have been tested for cytotoxic activity on huCHO FLT3 positive cells, huCHO CLL1 positive cells and double positive cells (DT): 1CL1 9-G4 CC x I2C0 x scFc x FL 4-E9 CC (X9G; format 1, with CLL1 TAA binding domain CD3 effector binding domain scFc HLE domain and FLT3 TAA domain in N to C order) CL1 9-G4 CC x FL 4-E9 CC x I2C0 x ScFc (C8O; format 2, with CLL1 TAA binding domain, FLT3 TAA domain, CD3 effector binding domain and scFc HLE domain in N to C order, SEQ ID NO: 3736), and CL1 9-G4 CC x ScFc x 4-E9 CC x I2C0 (Q1Y, format 3, with CLL1 TAA binding domain, scFc HLE domain, FLT3 TAA domain, and CD3 effector binding domain in N to C order). Results are represented in FIG. 24 and Table 15.

TABLE 15
Activity of dual targeting CLLxFLT3 antigen binding molecules with
EC50 values obtained on huCHO FLT3+ cells, huCHO CLL1+ cells and
double positive (DT 50:50) cells (from top to bottom, respectively).

| | |
|---|---|
| EC50 [pM] | factor |

CL1 9-G4 CC × I2CO × scFc × FL 4-E9 CC (X9G)
Format 1

| | |
|---|---|
| 457.3 | 79.5 |
| 202.8 | 35.2 |
| 5.8 | |

CL1 9-G4 CC × FL 4-E9 CC × I2CO × ScFc (C8O)
Format 2

| | |
|---|---|
| 197.5 | 23.3 |
| 291.2 | 34.4 |
| 8.5 | |

CL1 9-G4 CC × ScFc × 4-E9 CC × I2C0 (Q1Y)
Format 3

| | |
|---|---|
| 212.8 | 18.1 |
| 6383.2 | 542.0 |
| 11.8 | |

As it can be seen from the results, a selectivity gap can be achieved by dual targeting molecules of all three formats. Dual targeting antigen-binding molecules as described herein feature EC50 values of about 10 μM or below on cells positive for both targets while the such dual targeting molecules do not reach two digit pM EC50 values when employed with mono-targeting cells. This finding suggests that multitargeting molecules of the present invention do have selectivity gaps in terms of activity of at least factor 10, preferably at least factor 20 or even 30, which can beneficially be used to specifically address pathogenic target cells which express both targets and which can be bound at the same time by said molecules in order to trigger T-cell mediated cytotoxicity. Off-target toxicities and related side effects can thereby be reduced and a safer therapy can be provided based on the instantly described concept.

### Example 20 Stability of Multispecific Antigen-Binding Molecules with Two Target Binding Domains Versus Less Complex Bispecific Antigen-Binding Molecules with One Target Binding Domain

Thermal stability, monomer decrease after storage, monomer percentage after freeze thaw cycles and protein homogeneity were determined. Compared were either (i) CS1xBCMA multispecific antigen-binding molecules (mean including SEQ ID NO 1437) with (ii) CS1 bispecific antigen-binding molecules (mean of 28 constructs, including SEQ ID Nos 906 and 1401) and (iii) BCMA bispecific antigen-binding molecule (mean SEQ ID Nos 1412, 1423, 1434) or (iv) CD123xFLT3 multispecific antigen-binding molecules (mean of 46 constructs including SEQ ID Nos 673, 835, 838 and 871) with (v) FLT3 bispecific antigen-binding molecules (including SEQ ID Nos 648 and 670). The CD3 binder and the half-life extending scFc domain are identical, respectively.

Thermostability Measurement

Thermostability is measured by means of aggregation in dependence of temperature by dynamic light scattering (DLS). The bispecific antigen-binding molecule is provided in a buffer comprising citric acid, lysin-HCL, 4% trehalose at pH 7.0. The 200 μl of buffer comprising the bispecific antigen-binding molecule are transferred to a 96 well plate (Greiner bio-one) and measured by a Plate Reader II (Wyatt) while a temperature ramp at a rate of 0.04° C./min s run in the range from 40° C. to 70° C.

Monomer Decrease after 7 Day Storage

One sample (150 μl) comprising the construct to be analysed in a buffer at pH 7.0 is analysed by HP-SEC without storage. Two further samples are stored at 37° C. for 7 days. Thereafter, turbidity is determined at OD340 and the samples are analyed by HP-SEC (T7). The results are compared to determine a percental decrease.

Monomer Percentage after 3 Freeze/Thaw Cycles

One sample (150 μl) comprising the construct to be analysed in a buffer at pH 7.0 is analysed by UPLC (Waters) and for turbidity at OD340 without any freeze/thaw cycle. Two further samples are frozen at −80° C. are thawed and re-frozen to arrive at three freeze-thaw cycles. One cycle takes 30 min. Thereafter, turbidity is determined at OD340 and the samples are analyed by UPLC. The results are compared to determine a percental value, respectively.

Protein Homogeneity

Different folding states of antigen-binding molecules are determined by cation exchange chromatography (CIEX), wherein a high percental main peak stands for high protein homogeneity. 40 μl of sample (construct in buffer comprising citric acid, lysin, 4% trehalose at pH 7) are measured by a UPLC 7 (column Agilent Bi SCX, NP5, PK guard).

Results

TABLE 16
CS1 × BCMA and BC × CS1 multispecific antigen-binding molecule
improved stability

| | CS1 × BCMA/ | | |
|---|---|---|---|
| | BCMA × | CS1 | BCMA |
| Tested parameter | CS1 mean | mean | mean |
| Thermal Stability [° C.] | 56.2 | 54.2 | 52.0 |
| Monomer Decrease after 7 Day | 0.2 | 0.9 | 0.8 |
| Storage [%] | | | |
| Monomer Percentage after 3 Freeze/ | 100.0 | 99.6 | 100.0 |
| Thaw Cycles [% [symbol] | | | |
| Protein Homogeneity CIEX [% Main | 100.0 | 88.4 | 75.8 |
| Peak] | | | |

[symbol]

indicates data missing or illegible when filed

TABLE 17
CD123 × FLT3 multitargeting antigen-binding molecule improved
stability

| | CD123 × | |
|---|---|---|
| | FLT3 | FLT3 |
| Tested parameter | mean | mean |
| Thermal Stability [° C.] | 51.1 | 52.1 |
| Monomer Decrease after 7 Day Storage [%] | 0.00 | 4.1 |
| Monomer Percentage after 3 Freeze/Thaw Cycles [% [symbol] | 100 | 96.3 |
| Protein Homogeneity CIEX [% Main Peak] | 93.7 | 61.4 |

[symbol]

indicates data missing or illegible when filed

Lengthy table referenced here
US20220403035A1-20221222-T00001
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20220403035A1). An electronic copy of the table will also be available from the USPTO upon request and payment of the fee set forth in 37 CFR 1.19(b)(3).

1. A multitargeting antigen-binding molecule comprising at least three binding domains, wherein
(i) the first binding domain comprises a paratope which immuno-specifically binds to a first target cell surface antigen (TAA1),
(ii) the second binding domain comprises a paratope which immuno-specifically binds to a second target cell surface antigen (TAA2), and
(iii) the third binding domain comprises a paratope which immune-specifically binds to an extracellular epitope of the human and/or the Macaca CD3ε chain,
wherein the first, second and third binding domain are arranged in an amino to carboxyl order, and wherein the first binding domain and the second binding domain are linked by a peptide linker having a length of 5 to 24, preferably 18 amino acids.

2. The multitargeting antigen-binding molecule of claim 1, wherein the antigen-binding molecule comprises a fourth domain which comprises two polypeptide monomers, each comprising a hinge, a CH2 and a CH3 domain, wherein said two polypeptide monomers are fused to each other via a peptide linker
wherein said forth domain preferably comprises in an amino to carboxyl order:
hinge-CH2-CH3-linker-hinge-CH2-CH3
and/or wherein preferably each of said polypeptide monomers in the fourth domain has an amino acid sequence that is at least 90% identical to a sequence selected from the group from the group consisting of: SEQ ID NO: 17-24, wherein preferably each of said polypeptide monomers has an amino acid sequence selected from SEQ ID NO: 17-24,
and/or wherein preferably the CH2 domain comprises an intra domain cysteine disulfide bridge,
and/or wherein the first, second, third and fourth binding domain are arranged in an amino to carboxyl order.

3. The multitargeting antigen-binding molecule of any of the preceding claims, wherein the antigen-binding molecule is a single chain antigen-binding molecule, preferably a multitargeting scFv antigen-binding molecule.

4. The multitargeting antigen-binding molecule of any of the preceding claims, wherein the peptide linker between the first binding domain and the second binding domain is selected from having a length of 5, 6, 7, 8, 9, 10, 11, 12, 13, 14, 15, 16, 17, 18, 19, 20, 21, 22 23, or 24 amino acids, preferably 5, 6, 7, 8, 9, 10, 11 or 12 amino acids, more preferably 6 amino acids.

5. The multitargeting antigen-binding molecule of any of the preceding claims, wherein the peptide linker between the first binding domain and the second binding domain is selected from the group consisting of S(G₄S)ₙ, (G₄S)ₙ, G₄ₙ, and G₅ₙ, wherein n equals 1, 2, 3 or 4, preferably n equals 1 or 2, more preferably SG₄S.

6. The multitargeting antigen-binding molecule of any of the preceding claims, wherein any of TAA1 and TAA2 is selected from the group consisting of CS1, BCMA, FLT3, CD123, CD20, CD22, EpCAM, MSLN, CDH3 and CLL1,
and/or wherein TAA1 and TAA2 are preferably not identical.

7. The multitargeting antigen-binding molecule of any of the preceding claims, wherein the first binding domains is capable of binding to TAA1 and the second binding domain is capable of binding to TAA2 simultaneously, preferably wherein TAA1 and TAA2 are on the same target cell.
and/or wherein TAA1 and TAA2, respectively, are preferably selected from the group consisting of CS1 and BCMA, BCMA and CS1, FLT3 and CD123, CD123 and FLT3, CD20 and CD22, CD22 and CD20, EpCAM and MSLN, MSLN and EpCAM, MSLN and CDH3, CDH3 and MSLN, FLT3 and CLL1, and CLL1 and FLT3.

8. The multitargeting antigen-binding molecule of claim 2, wherein
(i) the first and second domain comprise two antibody variable domains and the third domain comprises two antibody variable domains;
(ii) the first and second domain comprise one antibody variable domain and the third domain comprises two antibody variable domains;
(iii) the first and second domain comprise two antibody variable domains and the third domain comprises one antibody variable domain; or
(iv) the first domain comprises one antibody variable domain and the second domain comprises one antibody variable domain,
and/or wherein the first, second and third domain are preferably fused to the fourth domain via a peptide linker.

9. The multitargeting antigen-binding molecule according to any of the preceding claims, wherein the antigen-binding molecule comprises in an amino to carboxyl order:
(a) the first domain;
(b) a peptide linker preferably having an amino acid sequence selected from the group consisting of SEQ ID NOs: 1-4 and 9-12, preferably 11;
(c) the second domain,
(d) a peptide linker preferably having an amino acid sequence selected from the group consisting of SEQ ID NOs: 1-3; and
(e) the third domain,

10. The multitargeting antigen-binding molecule according to any of the preceding claims, wherein the antigen-binding molecule further comprises in an amino to carboxyl order:
(f) a peptide linker having an amino acid sequence selected from the group consisting of SEQ ID NOs: 1, 2, 3, 9, 10, 11 and 12.
(e) the first polypeptide monomer of the fourth domain;
(f) a peptide linker having an amino acid sequence selected from the group consisting of SEQ ID NOs: 5, 6, 7 and 8; and
(g) the second polypeptide monomer of the fourth domain.

11. The multitargeting antigen-binding molecule according to according to any of the preceding claims, wherein the first and second binding domain comprise a VH region comprising CDR-H1, CDR-H2 and CDR-H3 selected from the group consisting of SEQ ID Nos: 33 to 35, 44 to 46, 55 to 57, 66 to 68, 77 to 79, 88 to 90, 99 to 101, 110 to 112, 121 to 123, 132 to 134, 143 to 145, 154 to 156, 165 to 167, 176 to 178, 187 to 189, 198 to 200, 209 to 211, 220 to 222, 231 to 233, 242 to 244, 253 to 255, 264 to 266, 275 to 277, 286 to 288, 297 to 299, 308 to 310, 319 to 321, 330 to 332, 341 to 343, 352 to 354, 363 to 365, 374 to 376, 385 to 387, 396 to 398, 407 to 409, 418 to 420, 429 to 431, 440 to 442, 451 to 453, 462 to 464, 473 to 475, 484 to 486, 495 to 497, 506 to 508, 517 to 519, 528 to 530, 539 to 541, 550 to 552, 561 to 563, 572 to 574, 583 to 585, 594 to 596, 605 to 607, 616 to 618, 627 to 629, 638 to 640, 649 to 651, 660 to 662, 896 to 898, 907 to 909, 918 to 920, 929 to 931, 940 to 942, 951 to 953, 962 to 964, 973 to 975, 984 to 986, 995 to 997, 1006 to 1008, 1017 to 1019, 1028 to 1030, 1039 to 1041, 1050 to 1052, 1061 to 1063, 1072 to 1074, 1083 to 1085, 1094 to 1096, 1105 to 1107, 1116 to 1118, 1127 to 1129, 1138 to 1140, 1149 to 1151, 1160 to 1162, 1171 to 1173, 1182 to 1184, 1193 to 1195, 1204 to 1206, 1215 to 1217, 1226 to 1228, 1237 to 1239, 1248 to 1250, 1259 to 1261, 1270 to 1272, 1281 to 1283, 1292 to 1294, 1303 to 1305, 1314 to 1316, 1325 to 1327, 1336 to 1338, 1347 to 1349, 1358 to 1360, 1369 to 1371, 1380 to 1382, 1391 to 1393, 1402 to 1404, 1413 to 1415, 1424 to 1426, 1489 to 1491, 1500 to 1502, 1511 to 1513, 1522 to 1524, 1533 to 1535, 1544 to 1546, 1555 to 1557, 1566 to 1568, 1577 to 1579, 1588 to 1590, 1599 to 1601, 1610 to 1612, 1621 to 1623, 1632 to 1634, 1643 to 1645, 1654 to 1656, 1827 to 1829, 1840 to 1842, 1853 to 1855, 1866 to 1868, 1879 to 1881, 1892 to 1894, 11905 to 1907, 1922 to 1924, 1935 to 1937, 1948 to 1950, 1961 to 1963, 1974 to 1976, 1987 to 1989, 2000 to 2002, 2013 to 2015, 2026 to 2028, 2039 to 2041, 2052 to 2054, 2065 to 2067, 2078 to 2080, 2091 to 2093, 2104 to 2106, 2117 to 2119, 2130 to 2132, 2143 to 2145, 2156 to 2158, 2169 to 2171, 2182 to 2184, 2195 to 2197, 2208 to 2210, 2221 to 2223, 2234 to 2236, 2247 to 2249, 3346 to 3348, 3357 to 3359, 3368 to 3370, 3379 to 3381, 3390 to 3392, 3401 to 3403, 3412 to 3414, 3423 to 3425, 3434 to 3436, 3445 to 3447, 3456 to 3458, 3467 to 3469, 3478 to 3480, 3489 to 3491, 3500 to 3502, 3511 to 3513, 3522 to 3524, 3533 to 3535, 3544 to 3546, 3555 to 3557, 3566 to 3568, 3679 to 3681, 3690 to 3692, 3712 to 3714, 3723 to 3725.

12. The multitargeting antigen-binding molecule according to according to any of the preceding claims, wherein the first and second binding domain comprise a VL region comprising CDR-L1, CDR-L2 and CDR-L3 selected from CDR-L1, CDR-L2 and CDR-L3 selected from the group consisting of SEQ ID Nos: 36 to 38, 47 to 49, 58 to 60, 69 to 71, 80 to 82, 91 to 93, 102 to 104, 113 to 115, 124 to 126, 135 to 137, 146 to 148, 157 to 159, 168 to 170, 179 to 181, 190 to 192, 201 to 203, 212 to 214, 223 to 225, 234 to 236, 245 to 247, 256 to 258, 267 to 269, 278 to 280, 289 to 291, 300 to 302, 311 to 313, 322 to 324, 333 to 335, 344 to 346, 355 to 357, 366 to 368, 377 to 379, 388 to 390, 399 to 401, 410 to 412, 421 to 423, 432 to 434, 443 to 445, 454 to 456, 465 to 467, 476 to 478, 487 to 489, 498 to 500, 509 to 511, 520 to 522, 531 to 533, 542 to 544, 553 to 555, 564 to 566, 575 to 577, 586 to 588, 597 to 599, 608 to 610, 619 to 621, 630 to 632, 641 to 643, 652 to 654, 663 to 665, 899 to 901, 910 to 912, 921 to 923, 932 to 934, 943 to 945, 954 to 956, 965 to 967, 976 to 978, 987 to 989, 998 to 1000, 1009 to 1011, 1020 to 1022, 1031 to 1033, 1042 to 1043, 1053 to 1055, 1064 to 1066, 1075 to 1077, 1086 to 1088, 1097 to 1099, 1108 to 1110, 1119 to 1121, 1130 to 1132, 1141 to 1143, 1152 to 1154, 1163 to 1165, 1174 to 1176, 1185 to 1187, 1196 to 1198, 1207 to 1209, 1218 to 1220, 1229 to 1231, 1240 to 1242, 1251 to 1253, 1262 to 1264, 1273 to 1275, 1284 to 1286, 1295 to 1297, 1306 to 1308, 1317 to 1319, 1328 to 1330, 1339 to 1341, 1350 to 1352, 1361 to 1363, 1372 to 1374, 1383 to 1385, 1394 to 1396, 1405 to 1407, 1416 to 1418, 1427 to 1429, 1492 to 1494, 1503 to 1505, 1514 to 1516, 1525 to 1527, 1536 to 1538, 1547 to 1549, 1558 to 1560, 1569 to 1571, 1580 to 1582, 1591 to 1593, 1602 to 1604, 1613 to 1615, 1624 to 1626, 1635 to 1637, 1646 to 1648, 1657 to 1659, 1830 to 1832, 1843 to 1845, 1856 to 1858, 1869 to 1871, 1882 to 1884, 1895 to 1897, 1908 to 1910, 1925 to 1927, 1938 to 1940, 1951 to 1953, 1964 to 1966, 1977 to 1979, 1990 to 1992, 2003 to 2005, 2016 to 2018, 2029 to 2031, 2042 to 2044, 2055 to 2057, 2068 to 2070, 2081 to 2083, 2094 to 2096, 2107 to 2109, 2120 to 2122, 2133 to 2135, 2146 to 2148, 2159 to 2131, 2172 to 2174, 2085 to 2187, 2198 to 2200, 2211 to 2213, 2224 to 2226, 2237 to 2239, 2250 to 2252, 3349 to 3351, 3360 to 3362, 3371 to 3373, 3382 to 3384, 3393 to 3392, 3404 to 3406, 3415 to 3417, 3426 to 3428, 3437 to 3439, 3448 to 3450, 3459 to 3461, 3470 to 3472, 3481 to 3483, 3492 to 3494, 3503 to 3505, 3514 to 3516, 3525 to 3527, 3536 to 3538, 3547 to 3549, 3558 to 3560, 3569 to 3571, 3682 to 3684, 3693 to 3695, 3715 to 3717, and 3726 to 3728.

13. The multitargeting antigen-binding molecule according to according to any of the preceding claims, wherein the first and second binding domain comprise a VH region selected from the group consisting of SEQ ID Nos: 39, 50, 61, 72, 83, 94, 105, 116, 127, 138, 149, 160, 171, 182, 193, 204, 215, 226, 237, 248, 259, 270, 281, 292, 303, 314, 325, 336, 347, 358, 369, 380, 391, 402, 413, 424, 435, 446, 457, 468, 479, 490, 501, 512, 523, 534, 545, 556, 567, 578, 589, 600, 611, 622, 633, 644, 655, 666, 902, 913, 924, 935, 946, 957, 968, 979, 990, 1001, 1012, 1023, 1034, 1045, 1056, 1067, 1078, 1089, 1100, 1111, 1122, 1133, 1144, 1155, 1166, 1177, 1188, 1199, 1210, 1221, 1232, 1243, 1254, 1265, 1276, 1287, 1298, 1309, 1320, 1331, 1342, 1353, 1364, 1375, 1386, 1397, 1408, 1419, 1430, 1495, 1506, 1517, 1528, 1539, 1550, 1561, 1572, 1583, 1594, 1605, 1616, 1627, 1638, 1649, 1660, 1833, 1846, 1859, 1872, 1885, 1898, 1911, 1928, 1941, 1954, 1967, 1980, 1993, 2006, 2019, 2032, 2045, 2058, 2071, 2084, 2097, 2110, 2123, 2136, 2149, 2162, 2175, 2188, 2201, 2214, 2227, 2240, 2253, 3352, 3363, 3374, 3385, 3396, 3407, 3418, 3429, 3440, 3451, 3462, 3473, 3484, 3495, 3506, 3517, 3528, 3539, 3550, 3561, 3572, 3686, 3696, 3718, 3729.

14. The multitargeting antigen-binding molecule according to according to any of the preceding claims, wherein the first and second binding domain comprises a VL region selected from the group consisting of SEQ ID Nos: 40, 51, 62, 73, 84, 95, 106, 117, 128, 139, 150, 161, 172, 183, 194, 205, 216, 227, 238, 249, 260, 271, 282, 293, 304, 315, 326, 337, 348, 359, 370, 381, 392, 403, 414, 425, 436, 447, 458, 469, 480, 491, 502, 513, 524, 535, 546, 557, 568, 579, 590, 601, 612, 623, 634, 645, 656, 667, 903, 914, 925, 936, 947, 958, 969, 980, 991, 1002, 1013, 1024, 1035, 1046, 1057, 1068, 1079, 1090, 1101, 1112, 1123, 1134, 1145, 1156, 1167, 1178, 1189, 1200, 1211, 1222, 1233, 1244, 1255, 1266, 1277, 1288, 1299, 1310, 1321, 1332, 1343, 1354, 1365, 1376, 1387, 1398, 1409, 1420, 1431, 1496, 1507, 1518, 1529, 1540, 1551, 1562, 1573, 1584, 1595, 1606, 1617, 1628, 1639, 1650, 1661, 1834, 1847, 1860, 1873, 1886, 1899, 1912, 1929, 1942, 1955, 1968, 1981, 1994, 2007, 2020, 2033, 2046, 2059, 2072, 2085, 2098, 2111, 2124, 2137, 2150, 2163, 2176, 2189, 2202, 2215, 2228, 2241, 2254, 3353, 3364, 3375, 3386, 3397, 3408, 3419, 3430, 3441, 3452, 3463, 3474, 3485, 3496, 3507, 3518, 3529, 3540, 3551, 3562, 3573, 3685, 3697, 3719, and 3730.

15. The multitargeting antigen-binding molecule according to according to any of the preceding claims, wherein the first and/or second binding domain comprises a scFv sequence selected from the group consisting of SEQ ID Nos: 41, 52, 63, 74, 85, 96, 107, 118, 129, 140, 151, 162, 173, 184, 195, 206, 217, 228, 239, 250, 261, 272, 283, 294, 305, 316, 327, 338, 349, 360, 371, 382, 393, 404, 415, 426, 437, 448, 459, 470, 481, 492, 503, 514, 525, 536, 547, 558, 569, 580, 591, 602, 613, 624, 635, 646, 657, 668, 671, 674, 677, 680, 683, 686, 689, 692, 695, 698, 701, 704, 707, 710, 713, 716, 719, 722, 725, 728, 731, 734, 737, 740, 743, 746, 749, 752, 755, 758, 761, 764, 767, 770, 773, 776, 779, 782, 785, 788, 791, 794, 797, 800, 803, 806, 809, 812, 815, 818, 821, 824, 827, 830, 833, 836, 839, 842, 845, 848, 851, 854, 857, 860, 863, 866, 869, 872, 874, 876, 878, 880, 882, 884, 886, 888, 890, 892, 894, 904, 915, 926, 937, 948, 959, 970, 981, 992, 1003, 1014, 1025, 1036, 1047, 1058, 1069, 1080, 1091, 1102, 1113, 1124, 1135, 1146, 1157, 1168, 1179, 1190, 1201, 1212, 1223, 1234, 1245, 1256, 1267, 1278, 1289, 1300, 1311, 1322, 1333, 1344, 1355, 1366, 1377, 1388, 1399, 1410, 1421, 1432, 1435, 1438, 1441, 1444, 1447, 1450, 1453, 1456, 1459, 1462, 1465, 1468, 1471, 1474, 1477, 1480, 1483, 1486, 1497, 1508, 1519, 1530, 1541, 1552, 1563, 1574, 1585, 1596, 1607, 1618, 1629, 1640, 1651, 1662, 1665, 1668, 1671, 1674, 1677, 1680, 1683, 1686, 1689, 1692, 1695, 1698, 1701, 1704, 1707, 1710, 1713, 1716, 1719, 1722, 1725, 1728, 1731, 1734, 1737, 1740, 1743, 1746, 1749, 1752, 1755, 1758, 1761, 1764, 1767, 1770, 1773, 1776, 1779, 1782, 1785, 1788, 1791, 1794, 1797, 1800, 1803, 1806, 1809, 1812, 1815, 1818, 1821, 1824, 1835, 1848, 1861, 1874, 1887, 1900, 1913, 1930, 1943, 1956, 1969, 1982, 1995, 2008, 2021, 2034, 2047, 2060, 2073, 2086, 2099, 2112, 2125, 2138, 2151, 2164, 2177, 2190, 2203, 2216, 2229, 2242, 2255, 2264, 2265, 2274, 2275, 2284, 2285, 2294, 2295, 2304, 2305, 2314, 2315, 2324, 2325, 2334, 2335, 2344, 2345, 2354, 2355, 2364, 2365, 2374, 2375, 2384, 2385, 2394, 2395, 2404, 2405, 2414, 2415, 2424, 2425, 2434, 2435, 2444, 2445, 2454, 2455, 2464, 2465, 2474, 2475, 2484, 2485, 2494, 2495, 2504, 2505, 2514, 2515, 2524, 2525, 2534, 2535, 2544, 2545, 2554, 2555, 2564, 2565, 2574, 2575, 2584, 2585, 2594, 2595, 2604, 2605, 2614, 2615, 2624, 2625, 2634, 2635, 2644, 2645, 2654, 2655, 2664, 2665, 2674, 2675, 2684, 2685, 2694, 2695, 2704, 2705, 2714, 2715, 2724, 2725, 2734, 2735, 2744, 2745, 2754, 2755, 2764, 2765, 2774, 2775, 2784, 2785, 2794, 2795, 2804, 2805, 2814, 2815, 2824, 2825, 2834, 2835, 2844, 2845, 2854, 2855, 2864, 2865, 2874, 2875, 2884, 2885, 2894, 2895, 2904, 2905, 2914, 2915, 2914, 2925, 2934, 2935, 2944, 2945, 2954, 2955, 2964, 2965, 2974, 2975, 2984, 2985, 2994, 2995, 3004, 3005, 3014, 3015, 3024, 3025, 3034, 3035, 3044, 3045, 3054, 3055, 3064, 3065, 3074, 3075, 3084, 3085, 3094, 3095, 3104, 3105, 3114, 3115, 3124, 3125, 3134, 3135, 3144, 3145, 3154, 3155, 3164, 3165, 3174, 3175, 3184, 3185, 3194, 3195, 3204, 3205, 3214, 3215, 3224, 3225, 3234, 3235, 3244, 3245, 3254, 3255, 3264, 3265, 3274, 3275, 3284, 3285, 3294, 3295, 3304, 3305, 3314, 3315, 3324, 3325, 3334, 3335, 3354, 3365, 3376, 3387, 3398, 3409, 3420, 3431, 3442, 3453, 3464, 3475, 3486, 3497, 3508, 3519, 3530, 3541, 3552, 3563, 3574, 3577, 3580, 3583, 3586, 3589, 3592, 3595, 3598, 3601, 3604, 3607, 3610, 3613, 3616, 3619, 3622, 3625, 3628, 3631, 3634, 3637, 3640, 3643, 3646, 3649, 3652, 3655, 3658, 3661, 3664, 3667, 3670, 3673, 3676, 3687, 3698, 3701, 3706, 3720, 3731, 3734, 3737, 3740, 3749, and 3750, preferably 1399 or 1435.

16. The multitargeting antigen-binding molecule according to according to any of the preceding claims, wherein the construct comprises a first and/or second target binding domain together with a third effector binding domain, the two or three binding domains together having a sequence selected from the group consisting of SEQ ID Nos: 42, 53, 64, 75, 86, 97, 108, 119, 130, 141, 152, 163, 174, 185, 196, 207, 218, 229, 240, 251, 262, 273, 284, 295, 306, 317, 328, 339, 350, 361, 372, 383, 394, 405, 416, 427, 438, 449, 460, 471, 482, 493, 504, 515, 526, 537, 548, 559, 570, 581, 592, 603, 614, 625, 636, 647, 658, 669, 672, 675, 678, 681, 684, 687, 690, 693, 696, 699, 702, 705, 708, 711, 714, 717, 720, 723, 726, 729, 732, 735, 738, 741, 744, 747, 750, 753, 756, 759, 762, 765, 768, 771, 774, 777, 780, 783, 786, 789, 792, 795, 798, 801, 804, 807, 810, 813, 816, 819, 822, 825, 828, 831, 834, 837, 840, 843, 846, 849, 852, 855, 858, 861, 864, 867, 870 873, 875, 877, 879, 881, 883, 885, 887, 889, 891, 893, 895, 905, 916, 927, 938, 949, 960, 971, 982, 993, 1004, 1015, 1026, 1037, 1048, 1059, 1070, 1081, 1092, 1103, 1114, 1125, 1136, 1147, 1158, 1169, 1180, 1191, 1202, 1213, 1224, 1235, 1246, 1257, 1268, 1279, 1290, 1301, 1312, 1323, 1334, 1345, 1356, 1367, 1378, 1389, 1400, 1411, 1422, 1433, 1436, 1439, 1442, 1445, 1448, 1451, 1454, 1457, 1460, 1463, 1466, 1469, 1472, 1475, 1478, 1481, 1484, 1487, 1498, 1509, 1520, 1531, 1542, 1553, 1564, 1575, 1586, 1597, 1608, 1619, 1630, 1641, 1652, 1663, 1666, 1669, 1672, 1675, 1678, 1681, 1684, 1687, 1690, 1693, 1696, 1699, 1702, 1705, 1708, 1711, 1714, 1717, 1720, 1723, 1726, 1729, 1732, 1735, 1738, 1741, 1744, 1747, 1750, 1753, 1756, 1759, 1762, 1765, 1768, 1771, 1774, 1777, 1780, 1783, 1786, 1789, 1792, 1795, 1798, 1801, 1804, 1807, 1810, 1813, 1816, 1819, 1822, 1825, 1836, 1849, 1862, 1875, 1888, 1901, 1914, 1931, 1944, 1957, 1970, 1983, 1996, 2009, 2022, 2035, 2048, 2061, 2074, 2087, 2300, 2113, 2126, 2139, 2152, 2165, 2178, 2191, 2204, 2217, 2230, 2243, 2256, 2260, 2266, 2267, 2276, 2277, 2286, 2287, 2296, 2297, 2306, 2307, 2316, 2317, 2326, 2327, 2336, 2337, 2346, 2347, 2356, 2357, 2366, 2367, 2376, 2377, 2386, 2387, 2396, 2397, 2406, 2407, 2416, 2417, 2426, 2427, 2436, 2437, 2446, 2447, 2456, 2457, 2466, 2467, 2476, 2477, 2486, 2487, 2496, 2497, 2506, 2507, 2516, 2517, 2526, 2527, 2536, 2537, 2546, 2547, 2556, 2557, 2566, 2567, 2576, 2577, 2586, 2587, 2596, 2597, 2606, 2607, 2616, 2617, 2626, 2627, 2636, 2637, 2646, 2647, 2656, 2657, 2666, 2667, 2676, 2677, 2686, 2687, 2696, 2697, 2706, 2707, 2716, 2717, 2726, 2727, 2736, 2737, 2746, 2747, 2756, 2757, 2766, 2767, 2776, 2777, 2786, 2787, 2796, 2797, 2806, 2807, 2816, 2817, 2826, 2827, 2836, 2837, 2846, 2847, 2856, 2857, 2866, 2867, 2876, 2877, 2886, 2887, 2896, 2897, 2906, 2907, 2916, 2917, 2926, 2927, 2936, 2937, 2946, 2947, 2956, 2957, 2966, 2967, 2976, 2977, 2986, 2987, 2996, 2997, 3006, 3007, 3016, 3017, 3026, 3027, 3036, 3037, 3046, 3047, 3056, 3057, 3066, 3067, 3076, 3077, 3086, 3087, 3096, 3097, 3106, 3107, 3116, 3117, 3126, 3127, 3136, 3137, 3146, 3147, 3156, 3157, 3166, 3167, 3176, 3177, 3186, 3187, 3196, 3197, 3206, 3207, 3216, 3217, 3226, 3227, 3236, 3237, 3246, 3247, 3246, 3247, 3256, 3257, 3266, 3267, 3276, 3277, 3286, 3287, 3296, 3297, 3306, 3307, 3316, 3317, 3326, 3327, 3336, 3337, 3355, 3366, 3377, 3388, 3399, 3410, 3421, 3432, 3443, 3454, 3465, 3476, 3487, 3498, 3509, 3520, 3531, 3542, 3553, 3564, 3575, 3578, 3581, 3584, 3587, 3590, 3593, 3596, 3599, 3602, 3605, 3608, 3611, 3614, 3617, 3620, 3623, 3626, 3629, 3632, 3635, 3638, 3641, 3644, 3647, 3650, 3653, 3656, 3659, 3662, 3665, 3668, 3671, 3674, 3677, 3688, 3699, 3702, 3703, 3707, 3721, 3732, 3735, 3738, 3741, 3751, and 3752, preferably 1400 or 1436.

17. The multitargeting antigen-binding molecule according to according to any of the preceding claims, wherein the construct comprises a first, and/or second target binding domain together with a third effector binding domain and a fourth domain conferring extended half-life, the two or three binding domains and the forth domain together having a sequence selected from the group consisting of SEQ ID Nos: 43, 54, 65, 76, 87, 98, 109, 120, 131, 142, 153, 164, 175, 186, 197, 208, 219, 230, 241, 252, 263, 274, 285, 296, 307, 318, 329, 340, 351, 362, 373, 384, 395, 406, 417, 428, 439, 450, 461, 472, 483, 494, 505, 516, 527, 538, 549, 560, 571, 582, 593, 604, 615, 626, 637, 648, 659, 670, 673, 676, 679, 682, 685, 688, 691, 694, 697, 700, 703, 706, 709, 712, 715, 718, 721, 724, 727, 730, 733, 736, 739, 742, 745, 748, 751, 754, 757, 760, 763, 766, 769, 772, 775, 778, 781, 784, 787, 790, 793, 796, 799, 802, 805, 808, 811, 814, 817, 820, 823, 826, 829, 832, 835, 838, 841, 844, 847, 850, 853, 856, 859, 862, 865, 868, 871, 906, 917, 928, 939, 950, 961, 972, 983, 994, 1005, 1016, 1027, 1038, 1049, 1060, 1071, 1082, 1093, 1104, 1115, 1126, 1137, 1148, 1159, 1170, 1181, 1192, 1203, 1214, 1225, 1236, 1247, 1258, 1269, 1280, 1291, 1302, 1313, 1324, 1335, 1346, 1357, 1368, 1379, 1390, 1401, 1412, 1423, 1434, 1437, 1440, 1443, 1446, 1449, 1452, 1455, 1458, 1461, 1464, 1467, 1470, 1473, 1476, 1479, 1482, 1485, 1488, 1499, 1510, 1521, 1532, 1543, 1554, 1565, 1576, 1587, 1598, 1609, 1620, 1631, 1642, 1653, 1664, 1667, 1670, 1673, 1676, 1679, 1682, 1685, 1688, 1691, 1694, 1697, 1700, 1703, 1706, 1709, 1712, 1715, 1718, 1721, 1724, 1727, 1730, 1733, 1736, 1739, 1742, 1745, 1748, 1751, 1754, 1757, 1760, 1763, 1766, 1769, 1772, 1775, 1778, 1781, 1784, 1787, 1790, 1793, 1796, 1799, 1802, 1805, 1808, 1811, 1814, 1817, 1820, 1823, 1826, 1838, 1851, 1864, 1877, 1890, 1903, 1916, 1933, 1946, 1959, 1972, 1985, 1998, 2011, 2024, 2037, 2050, 2063, 2076, 2089, 2102, 2115, 2128, 2141, 2154, 2167, 2180, 2194, 2206, 2219, 2232, 2245, 2258, 2262, 2270, 2271, 2280, 2281, 2290, 2291, 2300, 2301, 2310, 2311, 2320, 2321, 2330, 2331, 2340, 2341, 2350, 2351, 2360, 2361, 2370, 2371, 2380, 2381, 2390, 2391, 2400, 2401, 2410, 2411, 2420, 2421, 2430, 2431, 2440, 2441, 2450, 2451, 2460, 2461, 2470, 2471, 2480, 2481, 2490, 2491, 2500, 2501, 2510, 2511, 2520, 2521, 2530, 2531, 2540, 2541, 2550, 2551, 2560, 2561, 2570, 2571, 2580, 2581, 2590, 2591, 2600, 2601, 2610, 2611, 2620, 2621, 2630, 2631, 2640, 2641, 2650, 2651, 2660, 2661, 2670, 2671, 2680, 2681, 2690, 2691, 2700, 2701, 2710, 2711, 2720, 2721, 2730, 2731, 2740, 2741, 2750, 2751, 2760, 2761, 2770, 2771, 2780, 2781, 2790, 2791, 2800, 2801, 2810, 2811, 2820, 2821, 2830, 2831, 2840, 2841, 2850, 2851, 2860, 2861, 2870, 2871, 2880, 2881, 2890, 2891, 2900, 2901, 2910, 2911, 2920, 2921, 2930, 2931, 2940, 2941, 2950, 2951, 2960, 2961, 2970, 2971, 2980, 2981, 2990, 2991, 3000, 3001, 3010, 3011, 3020, 3021, 3030, 3031, 3040, 3041, 3050, 3051, 3060, 3061, 3070, 3071, 3080, 3081, 3090, 3091, 3100, 3101, 3110, 3111, 3120, 3121, 3130, 3131, 3140, 3141, 3150, 3151, 3160, 3161, 3170, 3171, 3180, 3181, 3190, 3191, 3200, 3201, 3210, 3211, 3220, 3221, 3231, 3240, 3241, 3250, 3251, 3260, 3261, 3270, 3271, 3280, 3281, 3290, 3291, 3300, 3301, 3310, 3311, 3320, 3321, 3330, 3331, 3340, 3341, 3344, 3345, 3356, 3367, 3378, 3389, 3400, 3411, 3422, 3433, 3444, 3455, 3466, 3477, 3488, 3499, 3510, 3521, 3532, 3543, 3554, 3565, 3576, 3579, 382, 3585, 3588, 3591, 3594, 3597, 3600, 3603, 3606, 3609, 3612, 3615, 3618, 3621, 3624, 3627, 3630, 3633, 3636, 3639, 3642, 3645, 3648, 3651, 3654, 3657, 3660, 3663, 3666, 3669, 3672, 3675, 3678, 3689, 3700, 3704, 3705, 3708, 3709, 3710, 3711, 3722, 3733, 3736, 3739, 3744, 3747, 3748, 3756, 3757, 3761, and 3762, preferably 1401 or 1437.

18. A polynucleotide encoding an antigen-binding molecule as defined in any one of the preceding claims.

19. A vector comprising a polynucleotide as defined in claim 18.

20. A host cell transformed or transfected with the polynucleotide as defined in claim 18 or with the vector as defined in claim 19.

21. A process for the production of the bispecific antigen-binding molecule according to any of the preceding claims, said process comprising culturing a host cell as defined in claim 20 under conditions allowing the expression of the antigen-binding molecule as defined in any one of claims 1 to 17 and recovering the produced antigen-binding molecule from the culture.

22. A pharmaceutical composition comprising the bispecific antigen-binding molecule according to any one of claims 1 to 17, or produced according to the process of claim 21,
which is preferably stable for at least four weeks at about −20° C.

23. The multitargeting antigen-binding molecule according to any of the preceding claims, or produced according to the process of claim 21, for use in the prevention, treatment or amelioration of a disease selected from a proliferative disease, a tumorous disease, cancer or an immunological disorder,
preferably cancer,
more preferably multiple myeloma (MM), acute myeloid leukemia (AML), Non-Hodgkin lymphoma (NHL), Non-small-cell lung carcinoma (NSCLC) and Colorectal cancer (CRC).

24. A method for the treatment or amelioration of a proliferative disease, a tumorous disease, cancer, or an immunological disorder, comprising the step of administering to a subject in need thereof the bispecific antigen-binding molecule according to claim 1, or produced according to the process of claim 21, wherein the disease preferably is multiple myeloma (MM), acute myeloid leukemia (AML), Non-Hodgkin lymphoma (NHL), Non-small-cell lung carcinoma (NSCLC) and Colorectal cancer (CRC).

25. A kit comprising the multitargeting antigen-binding molecule according to any one of claims 1 to 17, or produced according to the process of claim 21, a polynucleotide as defined in claim 18, a vector as defined in claim 19, and/or a host cell as defined in claim 20.

26. A method for the treatment or amelioration of gastrointestinal cancer, comprising the step of administering to a subject in need thereof a multitargeting antigen-binding molecule directed against CS1, BCMA, CD20, CD22, FLT3, CD123, CLL1, MSLN, or EpCAM and CD3.

27. A multitargeting antigen-binding molecule directed against CS1, BCMA, CD20, CD22, FLT3, CD123, CLL1, MSLN, and/or EpCAM and CD3 for use in the treatment or amelioration of multiple myeloma (MM), acute myeloid leukemia (AML), Non-Hodgkin lymphoma (NHL), Non-small-cell lung carcinoma (NSCLC) and Colorectal cancer (CRC).
